(19) United States
(12) Patent Application Publication
(10) Pub. No.: US 2003/0013178 A1
(43) Pub. Date: Jan. 16, 2003

(54) DNA sequence surrounding the glucocerebrosidase gene

(75) Inventors: Edward I. Ginns, Bethesda, MD (US); Ellen Sidransky, Bethesda, MD (US); Suzanne L. Winfield, Rockville, MD (US); Nahid Tayebi, Potomac, MD (US); Brian M. Martin, Rockville, MD (US)

Correspondence Address: NEEDLE & ROSENBERG P C, 127 PEACHTREE STREET N E, ATLANTA, GA, 30303-1811, US

(73) Assignee: Department of Health and Human Sevices, c/o National Institutes of Health

(21) Appl. No.: 09/790,852

(22) Filed: Feb. 22, 2001

Related U.S. Application Data
(63) Continuation of application No. 09/173,207, filed on Oct. 15, 1998, now abandoned.

(51) Int. Cl. C12N 9/24; C07H 21/04

(52) U.S. Cl. 435/200; 435/69.1; 435/320.1; 536/23.2

(57) ABSTRACT

The present invention provides the discovery and isolation of the nucleotide sequence of the human clk2, the human propin1, and the human cote1 genes that are located within the glucocerebrosidase gene locus. Also provided by the present invention are proteins or polypeptides encoded by those genes, nucleic acids encoding those polypeptides, and antibodies to those proteins. Further provided by the present invention are nucleic acids of the same apparent molecular size as the propin1 and cote1 gene regions and which have the same restriction pattern as the wild-type genes.

### BACKGROUND OF THE INVENTION

1. Field of the Invention

This invention relates generally to genes and nucleic acids surrounding the glucocerebrosidase gene (GBA). The invention also generally relates to genes and nucleic acids encoding clk2, cote1 and propin1, and the proteins encoded therein.

2. Background Art

Gaucher disease, the inherited deficiency of the enzyme glucocerebrosidase (EC 3.2.1.45), is the most common lysosomal hydrolase deficiency. The gene for glucocerebrosidase (GBA) is located on chromosome 1q21 (Ginns et al. 1985. “Gene mapping and leader polypeptide sequence of human glucocerebrosidase: implications for Gaucher disease.” Proc. Natl. Acad. Sci. USA 82: 7101-7105.) and is composed of 11 exons (Horowitz et al. 1989. “The human glucocerebrosidase gene and pseudogene: structure and evolution.” Genomics 4:87-96.). A highly homologous pseudogene (psGBA) is located nearby (Choudary et al.1985. “Molecular cloning and analysis of the human -glucocerebrosidase gene.” DNA 4:74.), and has contributed significantly to the origin of mutations in glucocerebrosidase (Tsuji et al. 1987. “A mutation in the human glucocerebrosidase gene in neuronopathic Gaucher's disease.” N. Engl. J. Med. 316: 570-575.). Cormand et al. have recently provided a localization of this region relative to 6 markers from the Généthon human linkage map (cormand et al. 1997. “Genetic fine localization of the -glucocerebrosidase (GBA) and prosaposin (PSAP) genes: implications for Gaucher disease.” Hum. Genet. 100:75-79.). Analyses of the mutations present in patients have revealed both single missense mutations (Beutler et al. 1997. “Hematologically important mutations: Gaucher disease” Blood Cells Mol. & Dis. 23:2-7.) and recombinant alleles, including several mutations which originate from the pseudogene sequence (Latham et al.1990. “Complex alleles of the acid-glucocerebrosidase gene in Gaucher disease.” Am. J. Hum. Genet. 47:79-86; Eyal et al. 1990. “Prevalent and rare mutations among Gaucher patients.” Gene 96: 277-283.). Patients have also been described with alleles resulting from a fusion between GBA and psGBA (Zimran et al 1990. “A glucocerebrosidase fusion gene in Gaucher disease. Implications for the molecular anatomy, pathogenesis, and diagnosis of this disorder.” J. Clin. Invest. 85: 219-222.).

Many attempts have been made to correlate patient genotypes with the clinical presentation of Gaucher disease. While there is some predictive value of certain alleles for either mild or severe disease (Zimran et al. 1989. “Prediction of severity of Gaucher's disease by identification of mutations at DNA level.” Lancet 2: 349-352; Beutler and Grabowski, 1995. “Gaucher disease. In The Metabolic Basis of inherited disease (eds. C. R. Scriver, A. L. Beaudet, W. S. Sly, D. Valle), pp. 2641-2670.” McGraw-Hill Information Services Co., Health Professions Division, New York), no specific symptom complex can be correlated with a unique genotype (Sidransky et al. 1994. “DNA mutational analysis of type 1 and type 3 Gaucher patients: How well do mutations predict phenotype? Hum. Mutat. 3: 25-28.). Based on clinical presentation, Gaucher disease has been divided into three types. Type 1 patients have very heterogeneous presentations, ranging from asymptomatic adults to young children with severe hepatosplenomegaly and bone involvement. Type 2 is invariably fatal, with infants classically developing symptoms at two to six months and dying by two years of age (Frederikson et al. 1972. “Glucosylceramide lipidoses: Gaucher's disease.” In The Metabolic Basis of inherited Disease (eds. J. B. Stanbury, J. B. Wyngarden, and D. S. Frederickson), pp.730-759. McGraw-Hill International Book Co., New York.). More recently, the severe phenotype of a knockout mouse model of Gaucher disease (Tybulewicz et al. 1992. “Animal model of Gaucher's disease from targeted disruption of the mouse glucocerebrosidase gene.” Nature 357: 407410.) prompted the recognition of a subset of severely affected type 2 patients which present and die in the perinatal period (Sidransky et al. 1992. “Gaucher disease in the neonate: A distinct Gaucher phenotype is analogous to a mouse model created by targeted disruption of the glucocerebrosidase gene.” Pediatr. Res. 32: 494498.). Type 3 is of intermediate severity, and includes patients with varying degrees of neurological impairment that develops in childhood or early adulthood.

A recent attempt to generate a point mutation mouse model of Gaucher disease led to the discovery of a novel gene, metaxin (MTX), which in the mouse is contiguous to and transcribed convergently to glucocerebrosidase. Metaxin shares a bidirectional promoter with the gene for thrombospondin 3. The insertion of a neomycin resistance cassette in the 3′ flanking region of GBA resulted in a knockout of the murine metaxin gene (Bornstein et al. 1995. “Metaxin, a gene contiguous to both thrombospondin 3 and glucocerebrosidase, is required for embryonic development in the mouse: Implications for Gaucher disease.” Proc. Natl. Acad. Sci. 92: 4547-4551.). Metaxin is a component of the protein translocation apparatus of the mitochondrial outer membrane (Armstrong et al. 1997. “Metaxin is a component of a preprotein import complex in the outer membrane of the mammalian mitochondrion.” J Biol Chem 272: 6510-6518.). Homozygosity for the metaxin knockout results in an embryonic lethal phenotype. Human metaxin is located downstream to psGBA and a pseudogene for metaxin was subsequently identified downstream to GBA in the intergenic region (Long et al. 1996. “Structure and organization of the human metaxin gene (MTX) and pseudogene.” Genomics 33; 177-184.). The region downstream to psGBA encodes for metaxin, thrombospondin 3, (Thbs3) (Vos et al. 1992. “Thrombospondin 3 (Thbs3), a new member of the thrombospondin gene family.” J. Biol. Chem. 267: 12192-12196.; Adolph et al 1995. “Structure and organization of the human thrombospondin 3 gene (THBS3).” Genomics 27: 329-336.), and polymorphic epithelial mucin 1 (Muc1) (Ligtenberg et al 1990. “Episialin, a carcinoma-associated mucin, is generated by a polymorphic gene encoding splice variants with alternative amino termini.” J. Biol. Chem. 265: 5573-5578.; Vos et al. 1995. “A tightly organized conserved gene cluster on mouse chromosome 3 (E3-F1).” Mamm. Genome 6: 820-822.). There is, therefore, an association of genes surrounding the glucocerebrosidase gene with clinical implications of Gaucher's disease. In order to elucidate these interactions, it is first necessary to discover the identity of the sequences in the area of the glucocerebrosidase gene. This invention provides that essential information.

This invention therefore provides the genomic DNA sequence and a more detailed organization of a 75 kb region around the glucocerebrosidase locus, including the duplicated region containing glucocerebrosidase and metaxin. Also provided are the origin and endpoints of the duplication leading to the pseudogenes for GBA and MTX. The invention further provides three new genes within the 32 kb of sequence upstream to GBA. Due to the close proximity of these three genes to GBA, common locus control, regulatory elements and overlapping transcripts may coordinately affect expression of these genes and, in the case of Gaucher's disease, influence the expression level of glucocerebrosidase. The potential involvement of contiguous gene effects could explain the more unusual phenotypes encountered among Gaucher patients. Of these three genes, the gene most distal to GBA is a protein kinase (clk2). The second gene,propin1, shares homology to a rat Secretory CArrier Membrane Protein (SCAMP37). Finally, the cote1 gene lies most proximal to GBA. Also provided are the nucleic acids encoding these polypeptides and the proteins encoded therein.

### SUMMARY OF THE INVENTION

The present invention provides the discovery and isolation of the nucleotide sequence of the human clk2, the human propin1, and the human cote1 genes that are located within the glucocerebrosidase gene locus.

Also provided by the present invention are proteins or polypeptides encoded by those genes, nucleic acids encoding those polypeptides, and antibodies to those proteins.

Further provided by the present invention are nucleic acids of the same apparent molecular size as the propin1 and cote1 gene regions and which have the same restriction pattern as the wild-type genes.

### BRIEF DESCRIPTION OF THE FIGURE

FIG. 1 shows a restriction map of the 5′ sequence flanking the glucocerebrosidase gene. The location of the clk2, propin1 and cote1 genes are indicated as follows: the 3′ end of clk2 corresponds to nucleic acid 10482, the 5′ and 3′ ends of propin1 correspond to nucleic acids 10950 and 17388, respectively, and the 5′ and 3′ ends of cote1 correspond to nucleic acids 17913 and 26165, respectively.

### DETAILED DESCRIPTION OF THE INVENTION

The present invention may be understood more readily by reference to the following detailed description of the preferred embodiments of the invention and the Example included therein.

Before the present compounds and methods are disclosed and described, it is to be understood that this invention is not limited to specific proteins, specific methods, or specific nucleic acids, as such may, of course, vary. It is also to be understood that the terminology used herein is for the purpose of describing particular embodiments only and is not intended to be limiting.

It must be noted that, as used in the specification and the appended claims, the singular forms “a,” “an,” and “the” include plural referents unless the context clearly dictates otherwise. Thus, for example, reference to “a nucleic acid” includes multiple copies of the nucleic acid and can also include more than one particular species of molecule.

In one aspect, the invention relates to a nucleic acid comprising the nucleic acid set forth in the Sequence Listing as SEQ ID NO: 1. The nucleic acid encodes the genes for human clk2, human propin1, human cote1, human GBA, human psMTX, human psGBA, human MTX and human Thbs3. The nucleic acid encodes human genes, which includes sequences both 5′ and 3′ to the coding regions of the genes. The nucleic acid set forth in SEQ ID NO: 1 represents the sequence of a genomic clone and therefore includes introns of the encoded genes.

In another aspect, the invention relates to a nucleic acid comprising the nucleic acid set forth in the Sequence Listing as any of SEQ ID NO: 2, SEQ ID NO: 4, and SEQ ID NO: 6 which correspond to the coding regions of the genes for human clk2, human propin1 and human cote1, respectively. These nucleic acids encode these respective genes, but do not include sequences both 5′ and 3′ to the coding region. The nucleic acid set forth in any of SEQ ID NO: 2, SEQ ID NO: 4 and SEQ ID NO: 6, therefore does not include any introns of these human genes.

The invention further provides a nucleic acid encoding the polypeptide set forth in any of SEQ ID NO: 3, SEQ ID NO: 5 and SEQ ID NO: 7, which are the clk2, propin1, and cote1 proteins encoded by the nucleic acids in SEQ ID NO: 2, 4, and 6. The invention further provides those nucleic acids encoding the proteins provided herein. Further provided by the present invention are genomic sequences corresponding to the propin1 unspliced RNA, the cote1 unspliced RNA, and the clk2 unspliced RNA, namely SEQ ID NO: 8, SEQ ID NO: 9, and SEQ ID NO: 10, respectively.

As used herein, the term “nucleic acid” refers to single-or multiple stranded molecules which may be DNA or RNA, or any combination thereof, including modifications to those nucleic acids. The nucleic acid may represent a coding strand or its complement, or any combination thereof Nucleic acids may be identical in sequence to the sequences which are naturally occurring for any of the novel genes discussed herein or may include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phophorothioate deoxynucleotides), selenium (yielding phosphorselenoate deoxynucleotides), or methyl groups (yielding methylphosphonate deoxynucleotides).

Similarly, one skilled in the art will recognize that compounds comprising the genes, nucleic acids, and fragments of the genes and nucleic acids as disclosed and contemplated herein are also provided. For example, a compound comprising a nucleic acid can be a derivative of a typical nucleic acid such as nucleic acids which are modified to contain a terminal or internal reporter molecule and/or those nucleic acids containing non-typical bases or sugars. These reporter molecules include, but are not limited to, isotopic and non-isotopic reporters. Therefore any molecule which may aid in detection, amplification, replication, expression, purification, uptake, etc. may be added to the nucleic acid construct.

The term “gene” as used herein means a unit of heredity that occupies a specific locus on a chromosome as well as any sequences associated with the expression of that nucleic acid. For example, the gene includes any introns normally present within the coding region as well as regions preceding and following the coding region. Examples of these non-coding regions include, but are not limited to transcription termination regions, promoter regions, enhancer regions and modulation regions.

The regions upstream and downstream of GBA may act as promoter factors for GBA expression and may be involved in the pathophysiology of Gaucher's disease. Additionally, the regions flanking GBA may contain potential cis-active elements that are required for regulation of GBA gene transcription. The genomic locus described herein may also encode overlapping sense and antisense RNAs. Such transcripts may be involved in the regulation of GBA expression and may play a role in the development of Gaucher's disease, through the activity of the respective gene products alone, or in combination with the other gene products, typically those surrounding the GBA gene. Antisense control of sense transcripts may be exerted at the level of transcription, maturation, transport, stability and translation of GBA. For example, the 3′ UTR of GBA may contain a region of antisense complementarity to a region of clk2, propin1 or cote1, whereby antisense interaction regulates the activity of the respective gene. Where sufficient mutations within these genes occur, this interaction is then be disrupted causing GBA to be incorrectly transcribed, processed, transported, translated etc. Alternatively, the clk2, propin1, and cote1 genes may encode proteins that covalently or noncovalently associate with GBA. Mutations within the genes contemplated in this invention may therefore result in mutant proteins that are deficient in their ability to effect this protein-protein interaction.

To identify protein-protein interactions between the genes contemplated in this invention and GBA or other proteins, conventional yeast two hybrid assays can be utilized. These procedures employ commercially available kits (e.g., Matchmaker* from Clontech, Palo Alto, Calif.) and involve fusing the “bait” (in this example, all or part of clk2, propin1 or cote1) to a DNA binding protein, such as GAL4, and fusing the “target” (e.g. a cDNA library) to an activating protein, such as the activation element domain of VP-16. Both constructs are then transformed into yeast cells containing a selectable marker gene under the control of, in this example, a GAL4 binding element. Thus, only those yeast colonies containing cDNAs of proteins or protein fragments that interact with the GALA4-bait constructs will be detected. The cDNA of the target construct is isolated from positive clones and conventional methods used to isolate the cDNA encoding the protein or protein fragment that interacts with the GAL4-bait construct.

The genes and nucleic acids provided for by the present invention may be obtained in any number of ways. For example, a DNA molecule encoding clk2, propin1 or cote1 can be isolated from the organism in which it is normally found. For example, a genomic DNA or cDNA library can be constructed and screened for the presence of the gene or nucleic acid of interest. Methods of constructing and screening such libraries are well known in the art and kits for performing the construction and screening steps are commercially available (for example, Stratagene Cloning Systems, La Jolla, calif.). Once isolated, the gene or nucleic acid can be directly cloned into an appropriate vector, or if necessary, be modified to facilitate the subsequent cloning steps. Such modification steps are routine, an example of which is the addition of oligonucleotide linkers which contain restriction sites to the termini of the nucleic acid. General methods are set forth in Sambrook et at. “Molecular Cloning, a Laboratory Manual,” Cold Spring Harbor Laboratory Press (1989).

Another example of a method of obtaining a DNA molecule encoding a specific gene, CDS, mRNA or protein of the present invention is to synthesize a recombinant DNA molecule which encodes that protein. For example, oligonucleotide synthesis procedures are routine in the art and oligonucleotides coding for a particular protein region are readily obtainable through automated DNA synthesis. A nucleic acid for one strand of a double-stranded molecule can be synthesized and hybridized to its complementary strand. One can design these oligonucleotides such that the resulting double-stranded molecule has either internal restriction sites or appropriate 5′ or 3′ overhangs at the termini for cloning into an appropriate vector. Double-stranded molecules coding for relatively large proteins can readily be synthesized by first constructing several different double-stranded molecules that code for particular regions of the protein, followed by ligating these DNA molecules together. For example, Cunningham, et al. “Receptor and Antibody Epitopes in Human Growth Hormone Identified by Homolog-Scanning Mutagenesis,” Science, 243:1330-1336 (1989), have constructed a synthetic gene encoding the human growth hormone gene by first constructing overlapping and complementary synthetic oligonucleotides and ligating these fragments together. See also, Ferretti, et al. Proc. Nat. Acad. Sci. 82:599-603 (1986), wherein synthesis of a 1057 base pair synthetic bovine rhodopsin gene from synthetic oligonucleotides is disclosed. By constructing the desired sequence in this manner, one skilled in the art can readily obtain any particular protein such as clk2, propin1, or cote1, with desired amino acids at any particular position or positions within the protein. See also, U.S. Pat. No. 5,503,995 which describes an enzyme template reaction method of making synthetic genes. Techniques such as this are routine in the art and are well documented. These nucleic acids can then be expressed in vivo or in vitro as discussed below.

Once the gene or nucleic acid sequence of the desired gene is obtained, the sequence encoding specific amino acids can be modified or changed at any particular amino acid position by techniques well known in the art. For example, PCR primers can be designed which span the amino acid position or positions and which can substitute any amino acid for another amino acid. Then a nucleic acid can be amplified and inserted into the wild-type coding sequence in order to obtain any of a number of possible combinations of amino acids at any position of the gene. Alternatively, one skilled in the art can introduce specific mutations at any point in a particular nucleic acid sequence through techniques for point mutagenesis. General methods are set forth in Smith, “In vitro mutagenesis” Ann. Rev. Gen., 19:423-462 (1985) and Zoller, “New molecular biology methods for protein engineering” Curr. Opin. Struct Biol., 1:605-610 (1991). Techniques such as these can also be used to modify the genes or nucleic acids in regions other than the coding regions, such as the promoter regions or any regulatory or noncoding region.

As used herein, the term “isolated” refers to a nucleic acid separated or significantly free from at least some of the other components of the naturally occurring organism, for example, the cell structural components commonly found associated with nucleic acids in a cellular environment and/or other nucleic acids. The isolation of the native nucleic acids can be accomplished, for example, by techniques such as cell lysis followed by phenol plus chloroform extraction, followed by ethanol precipitation of the nucleic acids. The nucleic acids of this invention can be isolated from cells according to any of many methods well known in the art.

An isolated nucleic acid comprising a unique fragment of at least 10 nucleotides of the nucleic acid set forth in the Sequence Listing as any of SEQ ID NO: 1, SEQ ID NO: 2, SEQ ID NO: 4, SEQ ID: 6, SEQ ID: 8, SEQ ID: 9 or SEQ ID NO: 10 is also provided. Unique fragments, as used herein means a nucleic acid of at least 10 nucleotides that is not identical to any other known nucleic acid sequence. Examples of the sequences of at least 10 nucleotides that are unique to clk2, propin1 and cote1 can be readily ascertained by comparing the sequence of the nucleic acid in question to sequences catalogued in GenBank, or any other sequence database, using the computer programs such as DNASIS (Hitachi Engineering, Inc.) or Word Search or FASTA of the Genetics Computer Group (GCG) (Madison, Wis.), which search the catalogued nucleotide sequences for similarities to the nucleic acid in question. If the sequence does not match any of the known sequences, it is unique. For example, the sequence of nucleotides 1-10 can be used to search the databases for an identical match. If no matches are found, then nucleotides 1-10 represent a unique fragment. Next, the sequence of nucleotides 2-11 can be used to search the databases, then the sequence of nucleotides 3-13, and so on up to nucleotides 75260 to 75270 of the sequence set forth in the Sequence Listing as SEQ ID NO: 1. The same type of search can be performed for sequences of 11 nucleotides, 12 nucleotides, 13 nucleotides, etc. The possible fragments range from 10 nucleotides in length to 1 nucleotides less that the sequence set forth in the Sequence Listing as SEQ ID NO: 1. These unique nucleic acids, as well as degenerate nucleic acids can be used, for example, as primers for amplifying nucleic acids in order to isolate allelic variants of the clk2, propin1 or cote1 proteins or as primers for reverse transcription of clk2, propin1 or cote1 mRNA, or as probes for use in detection techniques such as nucleic acid hybridization. One skilled in the art will appreciate that even though a nucleic acid of at least 10 nucleotides is unique to a specific gene, that nucleic acid fragment can still hybridize to many other nucleic acids and therefore be used in techniques such as amplification and nucleic acid detection.

Also provided are allelic variants of the clk2, propin1 and cote1 proteins set forth in the Sequence Listing as any of SEQ ID NO: 3, SEQ ID NO: 5, and SEQ ID NO: 7. As used herein, the term “allelic variations” or “allelic variants” is used to describe the same, or similar proteins that are diverged from the clk2, propin1, or cote1 proteins set forth in SEQ ID NO: 3, SEQ ID NO: 5 and SEQ ID NO: 7 by less than 20% in their corresponding amino acid identity. In another embodiment, these allelic variants are less than 18% divergent in their corresponding amino acid identity. In another embodiment, these allelic variants are less than 15% divergent in their corresponding amino acid identity. In another embodiment, these allelic variants are less than 12% divergent in their corresponding amino acid identity. In another embodiment, these allelic variants are less than 10% divergent in their corresponding amino acid identity. In another embodiment, these allelic variants are less than 7% divergent in their corresponding amino acid identity. In another embodiment, these allelic variants are less than 5% divergent in their corresponding amino acid identity. In another embodiment, these allelic variants are less than 3% divergent in their corresponding amino acid identity. In another embodiment, these allelic variants are less than 2% divergent in their corresponding amino acid identity. In yet another embodiment, these allelic variants are less than 1% divergent in their corresponding amino acid identity. These amino acids can be substitutions within the amino acid sequence set forth in the Sequence Listing as any of SEQ ID NO: 3, SEQ ID NO: 5, and SEQ ID NO: 7, they can be deletions from the amino acid sequence set forth in the Sequence Listing as SEQ ID NO: 3, SEQ ID NO: 5, and SEQ ID NO: 7 and they can be additions to the amino acid sequence set forth in the Sequence Listing as SEQ ID NO: 3, SEQ ID NO: 5 and SEQ ID NO: 7, or any combinations thereof.

The homology between the protein coding regions of the nucleic acids encoding the allelic variants of the clk2, propin1 and the cote1 proteins is preferably less than 20% divergent from the region of the nucleic acid set forth in the Sequence Listing as SEQ ID NO: 2, SEQ ID NO: 4 and SEQ ID NO: 6, respectively, encoding the proteins. In another embodiment, the corresponding nucleic acids are less than 18% divergent in their sequence identity. In another embodiment, the corresponding nucleic acids are less than 15% divergent in their sequence identity. In another embodiment, the corresponding nucleic acids are less than 12% divergent in their sequence identity. In another embodiment, the corresponding nucleic acids are less than 10% divergent in their sequence identity. In another embodiment, corresponding nucleic acids are less than 7% divergent in their sequence identity. In another embodiment, the corresponding nucleic acids are less than 5% divergent in their sequence identity. In another embodiment, the corresponding nucleic acids are less than 3% divergent in their sequence identity. In another embodiment, the corresponding nucleic acids are less than 2% divergent in their corresponding sequence identity. In yet another embodiment, the corresponding nucleic acids are less than 1% divergent in their sequence identity

One skilled in the art will appreciate that nucleic acids encoding homologs or allelic variants of the proteins set forth in the Sequence Listing as SEQ ID NO: 3, SEQ ID NO: 5 and SEQ ID NO: 7 can be isolated in a manner similar to that used to isolate the nucleic acids set forth in the Sequence Listing of the present invention as SEQ ID NO: 1, SEQ ID NO: 2, SEQ ID NO: 4, SEQ D NO: 6, SEQ ID NO: 8, SEQ ID NO: 9, and SEQ ID NO: 10. For example, given the sequence of the primers used to amplify the nucleic acid set forth in the sequence listing as SEQ ID NO: 1, one can use these or similar primers to amplify a homologous gene from other sources.

For example, SEQ ID NO: 9 and SEQ ID NO: 10 represent unspliced mRNA for the propin1 and cote1 genes, respectively. Using the sequence information provided herein, one skilled in the art can obtain the sequence of a primer that will hybridize to the desired message of genomic DNA, either in an intron, in an exon, or both, such that corresponding RNAs or DNAs from other organisms can readily be detected, and/or isolated. An example of a similar cross-species detection is provided in the Example herein where cDNA molecules were hybridized to genomic DNA from various vertebrates and budding yeast to detect analogous genes in those organisms. One can therefore use a spliced or an unspliced message, or a corresponding DNA, or a fragment thereof, as a probe to detect homologous sequences in other organisms, or similar genes from other individuals from the same species.

The present invention also contemplates any unique fragment of these genes, such as those encoding domains of the proteins set forth in the Sequence Listing as SEQ ID NO: 3, SEQ ID NO: 5, and SEQ ID NO: 7 or of the nucleic acids set forth in any of SEQ ID NO: 1, SEQ ID NO: 2, SEQ ID NO: 4, SEQ ID NO: 6, SEQ ID NO: 8, SEQ ID NO: 9, and SEQ ID NO: 10. To be unique, the fragment must be of sufficient size to distinguish it from other known sequences, most readily determined by comparing any nucleic acid fragment to the nucleotide sequences in computer databases, such as GenBank. Such comparative searches are standard in the art. Typically, a unique fragment usefull as a primer or probe will be at least 20 to about 25 nucleotides in length depending upon the specific nucleotide content of the sequence. Additionally, fragments can be, for example, at least about 30, 40, 50, 75, 100, 200 or 500 nucleotides in length All of the genes, nucleic acids, and fragments of the genes and nucleic acids disclosed and contemplated herein can be single or multiple stranded, depending on the purpose for which it is intended.

Once a nucleic acid encoding a particular protein of interest, or a region of that nucleic acid, is constructed, modified, or isolated, that nucleic acid can then be cloned into an appropriate vector, which can direct the in vivo or in vitro synthesis of that wild-type and/or modified protein. The vector is contemplated to have the necessary functional elements that direct and regulate transcription of the inserted gene, or nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene. (See generally, Sambrook et al.).

There are numerous E. coli (Escherichia coli) expression vectors known to one of ordinary skill in the art which are useful for the expression of the nucleic acid insert. Other microbial hosts suitable for use include bacilli, such as Bacillus subtilis, and other enterobacteriaceae, such as Salmonella, Serratia, and various Pseudomonas species. In these prokaryotic hosts one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. The promoters will typically control expression, optionally with an operator sequence, and have ribosome binding site sequences for example, for initiating and completing transcription and translation If necessary, an amino terminal methionine can be provided by insertion of a Met codon 5′ and in-frame with the downstream nucleic acid insert. Also, the carboxy-terminal extension of the nucleic acid insert can be removed using standard oligonucleotide mutagenesis procedures.

Additionally, yeast expression can be used. There are several advantages to yeast expression systems. First, evidence exists that proteins produced in a yeast secretion systems exhibit correct disulfide pairing. Second, post-translational glycosylation is efficiently carried out by yeast secretory systems. The Saccharomyces cerevisiae pre-pro-alpha-factor leader region (encoded by the MF″-1 gene) is routinely used to direct protein secretion from yeast. (Brake, et al. “∝-Factor-Directed Synthesis and Secretion of Mature Foreign Proteins in Saccharomyces cerevisiae.” Proc. Nat. Acad. Sci., 81:4642-4646 (1984)). The leader region of pre-pro-alpha-factor contains a signal peptide and a pro-segment which includes a recognition sequence for a yeast protease encoded by the KEX2 gene: this enzyme cleaves the precursor protein on the carboxyl side of a Lys-Arg dipeptide cleavage signal sequence. The nucleic acid coding sequence can be fused in-frame to the pre-pro-alpha-factor leader region. This construct is then put under the control of a strong transcription promoter, such as the alcohol dehydrogenase I promoter or a glycolytic promoter. The nucleic acid coding sequence is followed by a translation termination codon which is followed by transcription termination signals. Alternatively, the nucleic acid coding sequences can be fused to a second protein coding sequence, such as Sj26 or -galactosidase, used to facilitate purification of the fusion protein by affinity chromatography. The insertion of protease cleavage sites to separate the components of the fusion protein is applicable to constructs used for expression in yeast. Efficient post translational glycosylation and expression of recombinant proteins can also be achieved in Baculovirus systems.

Mammalian cells permit the expression of proteins in an environment that favors important post-translational modifications such as folding and cysteine pairing, addition of complex carbohydrate structures, and secretion of active protein Vectors useful for the expression of active proteins in mammalian cells are characterized by insertion of the protein coding sequence between a strong viral promoter and a polyadenylation signal. The vectors can contain genes conferring hygromycin resistance, gentamicin resistance, or other genes or phenotypes suitable for use as selectable markers, or methotrexate resistance for gene amplification. The chimeric protein coding sequence can be introduced into a Chinese hamster ovary (CHO) cell line using a methotrexate resistance-encoding vector, or other cell lines using suitable selection markers. Presence of the vector DNA in transformed cells can be confirmed by Southern blot analysis. Production of RNA corresponding to the insert coding sequence can be confirmed by Northern blot analysis. A number of other suitable host cell lines capable of secreting intact human proteins have been developed in the art, and include the CHO cell lines, HeLa cells, mycloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, or lipofectin mediated transfection or electroporation may be used for other cellular hosts.

Alternative vectors for the expression of genes or nucleic acids in mammalian cells, those similar to those developed for the expression of human gamma-interferon, tissue plasminogen activator, clotting Factor VIII, hepatitis B virus surface antigen, protease Nexin1, and eosinophil major basic protein, can be employed. Further, the vector can include CMV promoter sequences and a polyadenylation signal available for expression of inserted nucleic acids in mammalian cells (such as COS-7).

Insect cells also permit the expression of mammalian proteins. Recombinant proteins produced in insect cells with baculovirus vectors undergo post-translational modifications similar to that of wild-type proteins. Briefly, baculovirus vectors useful for the expression of active proteins in insect cells are characterized by insertion of the protein coding sequence downstream of the Autographica californica nuclear polyhedrosis virus (AcNPV) promoter for the gene encoding polyhedrin, the major occlusion protein. Cultured insect cells such as Spodoptera frugiperda cell lines are transfected with a mixture of viral and plasmid DNAs and the viral progeny are plated. Deletion or insertional inactivation of the polyhedrin gene results in the production of occlusion negative viruses which form plaques that are distinctively different from those of wild-type occlusion positive viruses. These distinctive plaque morphologies allow visual screening for recombinant viruses in which the AcNPV gene has been replaced with a hybrid gene of choice.

Alternatively, the genes or nucleic acids of the present invention can be operatively linked to one or more of the functional elements that direct and regulate transcription of the inserted gene as discussed above and the gene or nucleic acid can be expressed. For example, a gene or nucleic acid can be operatively linked to a bacterial or phage promoter and used to direct the transcription of the gene or nucleic acid in vitro. A further example includes using a gene or nucleic acid provided herein in a coupled transcription-translation system where the gene directs transcription and the RNA thereby produced is used as a template for translation to produce a polypeptide. One skilled in the art will appreciate that the products of these reactions can be used in many applications such as using labeled RNAs as probes and using polypeptides to generate antibodies or in a procedure where the polypeptides are being administered to a cell or a subject.

Expression of the gene or nucleic acid, either in combination with a vector or operatively linked to an appropriate sequence, can be by either in vivo or in vitro. In vivo synthesis comprises transforming prokaryotic or eukaryotic cells that can serve as host cells for the vector. Alternatively, expression of the gene or nucleic acid can occur in an in vitro expression system. For example, in vitro transcription systems are commercially available which are routinely used to synthesize relatively large amounts of mRNA. In such in vitro transcription systems, the nucleic acid encoding the desired gene would be cloned into an expression vector adjacent to a transcription promoter. For example, the Bluescript II cloning and expression vectors contain multiple cloning sites which are flanked by strong prokaryotic transcription promoters. (Stratagene Cloning Systems, La Jolla, Calif.). Kits are available which contain all the necessary reagents for in vitro synthesis of an RNA from a DNA template such as the Bluescript vectors. (Stratagene Cloning Systems, La Jolla, Calif.). RNA produced in vitro by a system such as this can then be translated in vitro to produce the desired protein. (Stratagene Cloning Systems, La Jolla, Calif.).

If ex vivo methods are employed, cells or tissues can be removed and maintained outside the body according to standard protocols well known in the art. The nucleic acids of this invention can be introduced into the cells via any gene transfer mechanism, such as, for example, virus-mediated gene delivery, calcium phosphate mediated gene delivery, electroporation, microinjection or proteoliposomes. The transduced cells can then be infused (e.g., in a pharmaceutically acceptable carrier) or homotopically transplanted back into a subject per standard methods for the cell or tissue type. Standard methods are known for transplantation or infusion of various cells into a subject

The nucleic acids of this invention can also be utilized for in vivo gene therapy techniques. With regard to gene therapy applications, the nucleic acid can comprise a nucleotide sequence which encodes a gene product which is meant to function in the place of a defective gene product and restore normal function to a cell which functioned abnormally due to the defective gene product. Alternatively, the nucleic acid may encode a gene product which was not previously present in a cell or was not previously present in the cell at a therapeutic concentration, whereby the presence of the exogenous gene product or increased concentration of the exogenous gene product imparts a therapeutic benefit to the cell and/or to a subject. For example, the nucleic acid of this invention can include but is not limited to, a gene encoding a gene product involved in Gaucher's disease.

For in vivo administration, the cells can be in a subject and the nucleic acid can be administered in a pharmaceutically acceptable carrier. The subject can be any animal in which it is desirable to selectively express a nucleic acid in a cell. In a preferred embodiment, the animal of the present invention is a human. In addition, non-human animals which can be treated by the method of this invention can include, but are not limited to, cats, dogs, birds, horses, cows, goats, sheep, guinea pigs, hamsters, gerbils and rabbits, as well as any other animal in which selective expression of a nucleic acid in a cell can be carried out according to the methods described herein.

In the method described above which includes the introduction of exogenous DNA into the cells of a subject (i.e., gene transduction or transfection), the nucleic acids of the present invention can be in the form of naked DNA or the nucleic acids can be in a vector for delivering the nucleic acids to the cells for expression of the nucleic acid inside the cell. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as Lipofectin®, Lipofectamine® (GIBCO-BRL, Inc., Gaithersburg, Md.), Superfect® (Qiagen, Inc. Hilden, Germany) and Transfectam® (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a Sonoporation machine (ImaRx Pharmaceutical Corp., Tucson, Ariz.).

As one example, vector delivery can be via a viral system, such as a retroviral vector system which can package a recombinant retroviral genome. The recombinant retrovirus can then be used to infect and thereby deliver nucleic acid to the infected cells. The exact method of introducing the nucleic acid into mammalian cells is, of course, not limited to the use of retroviral vectors. Other techniques are widely available for this procedure including the use of adenoviral vectors, adeno-associated viral (AAV) vectors, lentiviral vectors, pseudotyped retroviral vectors, and pox virus vectors, such as vaccinia virus vectors. Physical transduction techniques can also be used, such as liposome delivery and receptor-mediated and other endocytosis mechanism. This invention can be used in conjunction with any of these or other commonly used gene transfer methods.

The nucleic acid and the nucleic acid delivery vehicles of this invention, (e.g., viruses; liposomes, plasmids, vectors) can be in a pharmaceutically acceptable carrier for in vivo administration to a subject By “pharmaceutically acceptable” is meant a material that is not biologically or otherwise undesirable, i.e., the material may be administered to a subject, along with the nucleic acid or vehicle, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The nucleic acid or vehicle may be administered orally, parenterally (e.g., intravenously), by intramuscular injection, by intraperitoneal injection, transdermally, extracorporeally, topically or the like. The exact amount of the nucleic acid or vector required will vary from subject to subject, depending on the species, age, weight and general condition of the subject, the severity or mechanism of any disorder being treated, the particular nucleic acid or vehicle used, its mode of administration and the like.

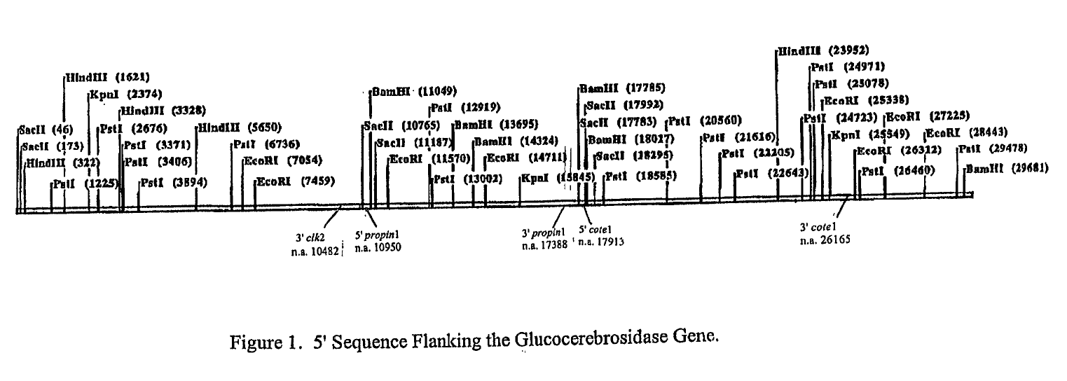

Also provided by the present invention is an isolated double-stranded nucleic acid, consisting of 1) a single-stranded DNA which has an apparent molecular size of 6.4 Kb and is derived from humans, and 2) a DNA complementary to the single-stranded DNA, giving the same restriction map as that obtained with the nucleic acid set forth in SEQ ID NO: 8, such as that of the corresponding region of the restriction map of FIG. 1, which represents a genomic nucleic acid corresponding to an unspliced RNA and therefore includes introns of a human gene encoding propin 1.

The term “apparent molecular size” as used herein means an estimated size obtained upon running the nucleic acid on a gel such as an agarose or polyacrylamide gel, or by other molecular size estimates techniques, such as molecular weight filtration. Briefly, the nucleic acid sample is applied to the gel and electrophoresed simultaneously with nucleic acid markers of known molecular size. Upon completion of gel electrophoresis, the size of the nucleic acid sample is estimated by comparing its mobility with the relative mobilities of the nucleic acid markers.

The invention further provides for a single-stranded RNA corresponding to the single-stranded DNA of the double-stranded nucleic acid set forth in SEQ ID NO: 8, and a single-stranded RNA corresponding to the complementary DNA of the double-stranded nucleic acid set forth in SEQ ID NO: 8.

The invention also provides an isolated double-stranded nucleic acid consisting of 1) a single-stranded DNA which has an apparent molecular size of 8.3 Kb and is derived from humans, and 2) a DNA complementary to the single-stranded DNA, giving the same restriction map as that obtained with the nucleic acid of SEQ ID NO: 9, such as that of the corresponding region of the restriction map of FIG. 1, which represents a genomic nucleic acid corresponding to an unspliced RNA and therefore includes introns of a human gene encoding cote 1.

The invention further provides for a single-stranded RNA corresponding to the single-stranded DNA of the double-stranded nucleic acid set forth in SEQ ID NO: 9, and a single-stranded RNA corresponding to the complementary DNA of the double-stranded nucleic acid set forth in SEQ ID NO: 9.

The invention further provides an isolated double-stranded nucleic acid, consisting of 1) a single-stranded DNA which has an apparent molecular size of 1.04 Kb and is derived from humans, and 2) a DNA complementary to the single-stranded DNA, which is a double-stranded nucleic acid corresponding to a cDNA of the genomic nucleic acid and therefore does not include any introns of a human gene encoding propin 1.

The invention also provides a single-stranded RNA corresponding to the single-stranded DNA of the double-stranded nucleic acid set forth in SEQ ID NO: 4, and a single-stranded RNA corresponding to the complementary DNA of the double-stranded nucleic acid set forth in SEQ ID NO: 4.

The invention also relates to an isolated double-stranded nucleic acid consisting of 1) a single-stranded DNA which has an apparent molecular size of 2.01 Kb and is derived from humans, and 2) a DNA complementary to the single-stranded DNA, which represents a cDNA of the genomic nucleic acid sequence and therefore does not include any introns of a human gene encoding cote 1.

Also provided by the present invention is a single-stranded RNA corresponding to the single-stranded DNA of the double-stranded nucleic acid set forth in SEQ ID NO: 6, and a single-stranded RNA corresponding to the complementary DNA of the double-stranded nucleic acid set forth in SEQ ID NO: 6.

The invention also provides an isolated double-stranded nucleic acid consisting of 1) a single-stranded DNA which has an apparent molecular size of 1.8 Kb and is derived from humans, and 2) a DNA complementary to the single-stranded DNA, and which is a variant of the nucleic acid encoding cote1.

Also provided by the present invention is a single-stranded RNA corresponding to the single-stranded DNA which has an apparent molecular size of 1.8 Kb and is derived from humans, and a single-stranded RNA corresponding to the complementary DNA of the single-stranded DNA.

The invention also relates to an isolated double-stranded nucleic acid consisting of 1) a single-stranded DNA which has an apparent molecular size of 6.0 Kb and is derived from humans, and 2) a DNA complementary to the single-stranded DNA, and which is a variant of the nucleic acid encoding cote 1.

The invention further provides a single-stranded RNA corresponding to the single-stranded DNA which has an apparent molecular size of 6.0 Kb and is derived from humans, and a single-stranded RNA corresponding to the complementary DNA of the single-stranded DNA.

The invention also relates to an isolated double-stranded nucleic acid consisting of 1) a single-stranded DNA which has a molecular size of 3.0 to 3.4 Kb and is derived from humans, and 2) a DNA complementary to the single-stranded DNA, and which is a variant of the nucleic acid encoding cote 1.

The invention further provides a single-stranded RNA corresponding to the single-stranded DNA which has an apparent molecular size of 3.0 to 3.4 Kb and is derived from humans, and a single-stranded RNA corresponding to the complementary DNA of the single-stranded DNA.

In another aspect, the invention provides a polypeptide encoded by the nucleic acid set forth in any of SEQ ID NO: 2, SEQ ID NO: 4, and SEQ ID NO: 6 and the polypeptides of any of SEQ ID NO: 3, SEQ ID NO: 5, and SEQ ID NO: 7, and the nucleic acids encoding the polypeptides of SEQ ID NO: 3, SEQ ID NO: 5, and SEQ ID NO: 7.

These polypeptides can also be obtained in any of a number of procedures well known in the art. One method of producing a polypeptide is to link two peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to a particular protein can be synthesized by standard chemical reactions. For example, a peptide or polypeptide can be synthesized and not cleaved from its synthesis resin whereas the other fragment of a hybrid peptide can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form a larger polypeptide. (Grant, “Synthetic Peptides: A User Guide,” W. H. Freeman and Co., N.Y. (1992) and Bodansky and Trost, Ed., “Principles of Peptide Synthesis,” Springer-Verlag Inc., N.Y. (1993)). Alternatively, the peptide or polypeptide can be independently synthesized in vivo as described above. Once isolated, these independent peptides or polypeptides may be linked to form a larger protein via similar peptide condensation reactions.

For example, enzymatic ligation of cloned or synthetic peptide segments can allow relatively short peptide fragments to be joined to produce larger peptide fragments, polypeptides or whole protein domains (Abrahmsen et al. Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. “Synthesis of Proteins by Native Chemical Ligation,” Science, 266:776-779 (1994)). The first step is the chemoselective reaction of an unprotected synthetic peptide-∝-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Clark-Lewis et al. FEBS Lett., 307:97 (1987), Clark-Lewis et al, J.Biol.Chem., 269:16075 (1994), Clark-Lewis et al. Biochemistry, 30:3128 (1991), and Rajarathnam et al. Biochemistry, 29:1689 (1994)).

Alternatively, unprotected peptide segments can be chemically linked where the bond formed between the peptide segments as a result of the chemical ligation is an unnatural (non-peptide) bond (Schnolzer et al. Science, 256:221(1992)). This technique has been used to synthesize analogs of protein domains as well as large amounts of relatively pure proteins with full biological activity (deLisle Milton et al. “Techniques in Protein Chemistry IV,” Academic Press, New York, pp. 257-267 (1992)).

The present invention also contemplates DNA probes for detecting the propin1 gene of the locus of SEQ ID NO: 1, wherein the DNA probe hybridizes to the nucleotide sequence set forth in the Sequence Listing as any of SEQ ID NO: 4 and/or SEQ ID NO: 8, and DNA probes for detecting the cote1 gene, wherein the DNA probe hybridizes to the nucleotide sequence set forth in the Sequence Listing as any of SEQ ID NO: 6 and/or SEQ ID NO: 9.

As used herein, the tern “DNA probe” refers to a nucleic acid fragment that selectively hybridizes under stringent conditions with a nucleic acid comprising a nucleic acid set forth in a sequence listed herein. This hybridization must be specific. The degree of complementarity between the hybridizing nucleic acid and the sequence to which it hybridizes should be at least enough to exclude hybridization with a nucleic acid encoding an unrelated protein.

Allelic variants can be identified and isolated by nucleic acid hybridization techniques. Probes selective to the nucleic acid set forth in the Sequence Listing as any of SEQ ID NO: 1, SEQ ID NO: 2, SEQ ID NO: 4, SEQ ID NO: 6, SEQ ID NO: 8, SEQ ID NO: 9 and SEQ ID NO: 10 can be synthesized and used to probe the nucleic acid from various cells, tissues, libraries etc. High sequence complementarity and stringent hybridization conditions can be selected such that the probe selectively hybridizes to allelic variants of the sequence set forth in the Sequence Listing as any of SEQ ID NO: 1, SEQ ID NO: 2, SEQ ID NO: 4, SEQ ID NO: 6, SEQ ID NO: 8, SEQ ID NO: 9 and SEQ ID NO: 10. For example, The selectively hybridizing nucleic acids of the invention can have at least 70%, 80%, 85%, 90%, 95%, 97%, 98% and 99% complementarity with the segment of the sequence to which it hybridizes. The nucleic acids can be at least 12, 50, 100, 150, 200, 300, 500, 750, or 1000 nucleotides in length Thus, the nucleic acid can be a coding sequence for the clk2, propin1 or cote1 proteins or fragments there of that can be used as a probe or primer for detecting the presence of these genes. If used as primers, the invention provides compositions including at least two nucleic acids which hybridize with different regions so as to amplify a desired region. Depending on the length of the probe or primer, target region can range between 70% complementary bases and full complementarity and still hybridize under stringent conditions. For example, for the purpose of diagnosing the presence of an allelic variant of the sequence set forth in the Sequence Listing as any of SEQ ID NO: 1, SEQ ID NO: 2, SEQ ID NO: 4, SEQ ID NO: 6, SEQ ID NO: 8, SEQ ID NO: 9 and SEQ ID NO: 10, the degree of complementarity between the hybridizing nucleic acid (probe or primer) and the sequence to which it hybridizes is at least enough to distinguish hybridization with a nucleic acid from other bacteria. The invention provides examples of nucleic acids unique to SEQ ID NO: 1, SEQ ID NO: 2, SEQ ID NO: 4, SEQ ID NO: 6, SEQ ID NO: 8, SEQ ID NO: 9 and SEQ ID NO: 10 in the Sequence Listing so that the degree of complementarity required to distinguish selectively hybridizing from nonselectively hybridizing nucleic acids under stringent conditions can be clearly determined for each nucleic acid.

“Stringent conditions” refers to the washing conditions used in a hybridization protocol. In general, the washing conditions should be a combination of temperature and salt concentration chosen so that the denaturation temperature is approximately 5-20° C. below the calculated Tₘ of the nucleic acid hybrid under study. The temperature and salt conditions are readily determined empirically in preliminary experiments in which samples of reference DNA immobilized on filters are hybridized to the probe or protein coding nucleic acid of interest and then washed under conditions of different stringencies. The Tₘ of such an oligonucleotide can be estimated by allowing 20° C. for each A or T nucleotide, and 4° C. for each G or C. For example, an 18 nucleotide probe of 50% G+C would, therefore, have an approximate Tₘ of 54° C.

Also provided herein are purified antibodies that selectively bind to the polypeptides provided and contemplated herein, or purified antibodies which selectively bind to a polypeptide encoded by the nucleic acid set forth in any of SEQ ID NO: 2, SEQ ID NO: 4, and SEQ ID NO: 6, and purified antibodies which selectively bind to a polypeptide encoded by a nucleic acid encoding the polypeptide set forth in any of SEQ ID NO: 3, SEQ ID NO: 5 and SEQ ID NO: 7. The antibody (either polyclonal or monoclonal) can be raised to any of the polypeptides provided and contemplated herein, in its naturally occurring form and in its recombinant form. The antibody can be used in techniques or procedures such as diagnostics, treatment, or vaccination. Antibodies can be made by many well-known methods (See, e.g. Harlow and Lane, “Antibodies; A Laboratory Manual” Cold Spring Harbor Laboratory, Cold Spring Harbor, N.Y., (1988)). Briefly, purified antigen can be injected into an animal in an amount and in intervals sufficient to elicit an immune response. Antibodies can either be purified directly, or spleen cells can be obtained from the animal. The cells can then fused with an immortal cell line and screened for antibody secretion. The antibodies can be used to screen nucleic acid clone libraries for cells secreting the antigen. Those positive clones can then be sequenced. (See, for example, Kelly et al. Bio/Technology, 10:163-167 (1992); Bebbington et al. Bio/Technology, 10:169-175 (1992)).

The phrase “specifically binds” with the polypeptide refers to a binding reaction which is determinative of the presence of the protein in a heterogeneous population of proteins and other biologics. Thus, under designated immunoassay conditions, the specified antibodies bound to a particular protein do not bind in a significant amount to other proteins present in the sample. Selective binding to an antibody under such conditions may require an antibody that is selected for its specificity for a particular protein. A variety of immunoassay formats may be used to select antibodies that selectively bind with a particular protein. For example, solid-phase ELISA immunoassays are routinely used to select antibodies selectively immunoreactive with a protein. See Harlow and Lane “Antibodies, A Laboratory Manual” Cold Spring Harbor Publications, New York, (1988), for a description of immunoassay formats and conditions that could be used to determine selective binding.

This invention also contemplates producing a selected cell line or a non-human transgenic animal model for the analysis of the function of a gene comprising introducing into an embryonic stem cell a vector having a selectable marker which, when the vector is inserted within a gene, the inserted vector can inhibit the expression of the gene, selecting embryonic stem cells expressing the selectable marker, excising the vector from the embryonic stem cells expressing the selectable marker such that host DNA from the gene is linked to the excised vector, sequencing the host DNA in the excised vector, comparing the sequence of the host DNA to known gene sequences to determine which host DNA is from a gene for which a model for the analysis of the function the gene is desired, selecting the embryonic stem cell containing the inhibited gene for which a model for the analysis of gene function is desired, and forming a cell line or a non-human transgenic animal from the selected embryonic stem cell.

It is also contemplated in this invention that transgenic animals can be produced which either overproduce the polypeptides of this invention or fail to produce the polypeptides of this invention in a functional form. For example, a transgenic animal which overproduces the propin1 of this invention can be produced according to methods well known in the art whereby nucleic acid encoding propin1 is introduced into embryonic stem cells, at which stage it is incorporated into the germline of the animal, resulting in the production of propin1 in the transgenic animal in increased amounts relative to a normal animal of the same species. One skilled in the art can determine if overproduction or under production of propin1 results in altered GBA expression.

A transgenic animal in which the expression of propin1, for example, is tissue specific is also contemplated for this invention. For example, transgenic animals that express or overexpress these genes at specific sites such as the brain can be produced by introducing a nucleic acid into the embryonic stem cells of the animal, wherein the nucleic acid is under the control of a specific promoter which allows expression of the nucleic acid in specific types of cells (e.g., a neuronal promoter which allows expression only in neuronal cells. One skilled in the art can determine if a tissue-specific alteration in clk2, propin1 or cote1 expression results in altered GBA expression by assaying for GBA expression in both the non-transgenic and the transgenic animal.

Alternatively, the transgenic animal of this invention can be a “knock out” animal (see, e.g., Willnow et al., 1996), which can be an animal that, for example, normally produces propin1 but has been altered to prevent the expression of the animal's nucleic acid which encodes propin1, thereby resulting in an animal which does not produce propin1 in a functional form. Such an animal may lack the ability to express all of the nucleic acids encoding propin1 or the transgenic animal may lack the ability to express some (one or more than one) but not all of the nucleic acids encoding the propin1.

For example, the transgenic “knock out” animal of this invention can have the expression of a gene or genes knocked out in specific tissues. This approach obviates viability problems that can be encountered if the expression of a widely expressed gene is completely abolished in all tissues. One skilled in the art could determine whether or not the “knock out” has influenced the expression of GBA by assaying for GBA in both the non-transgenic and transgenic animal. The knock-out mice can also be utilized to correlate particular genotypes with clinical presentation of Gaucher's disease.

The present invention is more particularly described in the following examples which are intended as illustrative only since numerous modifications and variations therein will be apparent to those skilled in the art.

### EXAMPLES

Isolation of Genomic Clones: The Center for Genetics in Medicine (CGM) YAC library (Burke and Olson 1991. “Preparation of clone libraries in yeast artificial-chromosome vectors.” In Methods in Enzymology. Guide to Yeast Genetics and Molecular Biology (eds. C. Guthrie, G. R Fink), pp. 251-270. Academic Press, San Diego, Calif.) was screened by PCR amplification of ordered arrays of pooled clones (Green and Olson 1990. “Systematic screening of yeast artificial-chromosome libraries by use of the polymerase chain reaction.” Proc. Natl. Acad. Sci.USA 87: 1213-1217.) using a primer pair specific for the human GBA gene. A YAC clone, CGY2981, contained both GBA and psGBA. A his3 mutation was introduced into the yeast from strain MB11, and the his5 mutation removed by standard mating procedures (Sherman 1991. “Getting started with yeast.” In Methods in Enzymology. Guide to Yeast Genetics and Molecular Biology (eds. C. Guthrie, G. R. Fink), pp. 3-21. Academic Press, San Diego, Calif.) The resulting strain, MNG1, was transformed with fragmentation vectors pBP108 and pBP109 (Pavan et al. 1991. “High-efficiency yeast artificial chromosome fragmentation vectors.” Gene 106:125-127.) to reduce the insert size. One of the derivatives, MNG4, with an insert of ˜85 kb, was confirmed to contain both GBA and psGBA and following partial Sau3AI digestion, was subcloned to Lambda, Dash (Stratagene, La Jolla, Ga.). The full-length YAC clone, MNG1, following partial SauA1 digestion, was also subcloned into SuperCos (Stratagene).

Several subclones that hybridized to a human 1.6-kb GBA cDNA were subcloned into pGEM7zf+ (Promega, Madison, Wis.) for sequencing. Plasmid subclones were ordered using sequence across the junction points in the DNA. A cosmid subclone of the YAC containing 16 kb of DNA upstream of GBA was also subcloned to pGEM7zf+ and pGEM11zf+ for sequencing.

Complementary DNA Clones: A human hippocampal cDNA (Lambda ZapII) was obtained from Stratagene. The human brain plasmid library in a pCMV-SPORT was from Life Technologies (Gaithersburg, Md.).

RACE PCR was used to determine the 5′ ends of cDNAs. PCR amplification was carried out using human brain cDNA ligated to an anchor (Clontech) or on cDNA isolated from the pSPORT human brain library with anchor or vector primers and nested gene-specific primers.

DNA Sequence Analysis: Plasmid and cosmid DNA were prepared by either standard alkaline lysis (Maniatis et al. 1982. “Molecular cloning: A laboratory manual.” Cold Spring Harbor Laboratory, Cold Spring Harbor, N.Y.), Perfect Prep kits (5 Prime →3 Prime, Boulder, Co.), or in some cases by CsCl gradient purification. Sequencing was performed on alkaline-denatured, double-stranded template using the Sequenase Reagent kit (U.S. Biochemical, Inc., Cleveland, Ohio) or on subclones using the Circumvent ThermoCycle Dideoxy DNA Sequencing kit (New England Biolabs, Beverly, Mass.) according to manufacturer's protocols, PCR products, cosmids, and subclones were sequenced using a Perkin-Elmer Applied Biosystems model 373A automated sequencer with the FS enzyme system and dye terminator chemistry according to the manufacturer's protocols (Perkin Elmer, Foster City, Calif.). Oligonucleotides were synthesized on an Expedite (Perseptive Biosystems, Framingham, Mass.) or Cyclone synthesizer (Milli. Bioresearch, Milford, Mass.) and used without purification. All sequence was determined in both directions, entered into PC-Gene and analyzed by GRAIL (Ubersbacher and Mural 1991. “locating protein-coding regions in human DNA sequences by a multiple sensor-neural network approach.” Proc. Natl. Acad. Sci. 88: 11261-11265.) to determine potential coding areas. A BLAST-N (Altschul et al. 1990. “Basic local alignment search tool.” J. Mol. Biol. 215: 403-410.) comparison of putative exon sequences with the GenBank database was used to identify homologies. The BLAST-X (Altschul et al. 1990) program was also used to compare the predicted amino acid sequence to the protein database.

Northern and Zooblot Hybridization: Multiple human tissue Northern blots and multiple species (Zoo) blots were obtained from Clontech Laboratories. Hybridization was carried out according to the manufacturer's protocol using cDNA from clk2 kinase, propin1, cote1, and -actin as probes.

The hybridization conditions used in the Northern and Zooblot hybridization experiments described herein can be used to detect nucleic acid species from other organisms that correspond to the clk2, propin1, and cote1 genes of the present invention. For example, a representative hybridization experiment would consist of prehybridizing the filter(s) for 1 to 4 hours at 42° C. in a prehybridization mix (50% v/v formamide, 5×SSC, 5×Denhardts, 20 mM Na phosphate pH 6.5, 1% v/v glycine, 100 μg/ml E.coli RNA and 10 μg/ml poly A RNA in sterile water). Following prehybridization, E.coli DNA is added to the probe using 100 μl per 10 ml of hybridization mix used. The probe/E.coli DNA is boiled for 5 minutes, then quick cooled on ice for 3-5 minutes. The probe/E.coli DNA is added to a hybridization mix (50% formamide, 5×SSC, 5×Denhardt's, 20 mM Na phosphate pH 6.5, 10% dextran sulfate and 10 μg/ml poly A RNA) and mixed gently. The filter is then hybridized overnight at 42° C. Subsequent to hybridization, the filters are washed 2×10 minutes at room temperature with 6×SSC, washed 4×5 minutes at room temperature with 2×SSC, 0.1% SDS, washed 2×15 minutes at room temperature with .01×SSC, 0.1% SDS, and washed 1×20 minutes at 55° C. with 0.1×SSC, 0.1% SDS.

Detailed Information Regarding the Glucocerebrosidase Gene Locus

The following information gives the GENBANK accession information regarding the sequence of the present invention, which includes the coordinates for the splice junctions of the unspliced RNAs and the coordinates for the start and the end of the RNAs of the genes within this locus:

LOCUS AF023268 75270 bp DNA PRI 28-OCT-1997
DEFINITION Homo sapiens clk2 kinase (CLK2), propin1, cote1, glucocerebrosidase

(GBA), and metaxin genes, complete cds; metaxin pseudogene and
glucocerebrosidase pseudogene; and thrombospondin3 (THBS3) gene,
partial cds.

ACCESSION AF023268
NID g2564910
KEYWORDS
SOURCE human.

ORGANISM Homo sapiens

Eukaryotae; Metazoa; Chordata; Vertebrata; Mammalia; Eutheria;
Primates; Catarrhini; Hominidae; Homo.

REFERENCE 1 (bases 1 to 75270)

AUTHORS Winfield, S. L., Tayebi, N., Martin, B. M., Ginns, E. I. and Sidransky, E.
TITLE Identification of three additional genes contiguous to the

glucocerebrosidase locus on chromosome 1q21: implications for
Gaucher disease

JOURNAL Genome Res. 7 (10), 1020-1026 (1997)
MEDLINE 97474796

REFERENCE 2 (bases 1 to 75270)

AUTHORS Winfield, S. L., Tayebi, N., Martin, B. M., Ginns, E. I. and Sidransky, E.
TITLE Direct Submission
JOURNAL Submitted (05-SEP-1997) Clinical Neuroscience Branch, NIMH, 9000

Rockville Pike Bldg. 49 Rm. B1EE16, Bethesda, MD 20892, USA

FEATURES Location/Qualifiers

source 1..75270

/organism=“Homo sapiens”
/db_xref =“taxon:9606”
/chromosome=“1”
/map=“1q21”

mRNA join(1..129,2354..2523,3624..3852,4550..4637,4987..5053,

5220..5336,6458..6624,7401..7495,8580..8709,8804..8886,
9055..9134,9315..9405,9956..10482)
/gene=“CLK2”

gene 1..10482

/gene=“CLK2”

CDS join(2354..2523,3624..3852,4550..4637,4987..5053,

5220..5336,6458..6624,7401..7495,8580..8709,8804..8886,
9055..9134,9315..9405,9956..10138)
/gene=“CLK2”
/codon_start=1
/product=“clk2 kinase”
/db_xref=“PID:g2564911”

/translation=“MPHPRRYHSSERGSRGSYREHYRSRKHKRRRSRSWSSSSDRTRR
RRREDSYHVRSRSSYDDRSSDRRVYDRRYCGSYRRNDYSRDRGDAYYDTDYRHSYEYQ
RENSSYRSQRSSRRKHRRRRRRSRTFSRSSSQHSSRRAKSVEDDAEGHLIYHVGDWLQ
ERYEIVSTLGEGTFGRVVQCVDHRRGGARVALKIIKNVEKYKEAARLEINVLEKINEK
DPDNKNLCVQMFDWFDYHGHMCISFELLGLSTFDFLKDNNYLPYPIHQVRHMAFQLCQ
AVKFLHDNKLTHTDLKPENILFVNSDYELTYNLEKKRDERSVKSTAVRVVDFGSATFD
HEHHSTIVSTRHYRAPEVILELGWSQPCDVWSIGCIIFEYYVGFTLFQTHDNREHLAM
MERILGPIPSRMIRKTRKQKYFYRGRLDWDENTSAGRYVRENCKPLRRYLTSEAEEHH
QLFDLIESMLEYEPAKRLTLGEALQHPFFARLRAEPPNKLWDSSRDISR”

mRNA join(10950..11272,11624..11701,12699..12821,12908..13028,

14412..14540,15709..15868,15980..16081,16574..16691,
16947..17388)
/product=“propin1”

CDS join(11207..11272,11624..11701,12699..12821,12908..13028,

14412..14540,15709..15868,15980..16081,16574..16691,
ATTORNEY DOCKET NO: 14014.0296
16947..17093)
/codon_start=1
/product=“propin1”
/db_xref=“PID:g2564915”

/translation=“MAQSRDGGNPFAEPSELDNPFQDPAVIQHRPSRQYATRDVYNPF
ETREPPPAYEPPAPAPLPPPSAPSLQPSRKLSPTEPKNYGSYSTQASAAAATAELLKK
QEELNRKAEELDRRERELQHAALGGTATRQNNWPPLPSFCPVQPCFFQDISMEIPQEF
QKTVSTMYYLWMCSTLALLLNFLACLASFCVETNNGAGFGLSILWVLLFTPCSFVCWY
RPMYKAFRSDSSFNFFAFFFNFFDQDVLFVLQAIGIPGWGFSGWISALVVPKGNTAVS
VLMLLVALLFTGIAVLGIVMLKRIHSLYRRTGASFQKAQQEFAAGVFSNPAVRTRAAN
AAAGAAENAFRAP”

mRNA join(17913..18715,18913..18969,19174..19284,19390..19509,

19636..19743,21463..21638,21770..21869,22207..22276,
22550..23095,24910..24978,25080..25333,25428..26165)
/product=“cote1”

CDS join(18491..18715,18913..18969,19174..19284,19390..19509,
19636..19743,21463..21638,21770..21869,22207..22276,
22550.23095,24910..24978,25080..25333,25428..25601)
/codon_start=1
/product=“cote1”
/db_xref=“PID:g2564916”

/translation=“MMPSPSDSSRSLTSRPSTRGLTHLRLHRPWLQALLTLGLVQVLL
GILVVTFSMVASSVTTTESIKRSCPSWAGFSLAFSGVVGIVSWKRPFTLVISFFSLLS
VLCVMLSMAGSVLSCKNAQLARDFQQCSLEGKVCVCCPSVPLLRPCPESGQELKVAPN
STCDEARGALKNLLFSVCGLTICAAIICTLSAIVCCIQIFSLDLVHTQLAPERSVSGP
LGPLGCTSPPPAPLLHTMLDLEEFVPPVPPPPYYPPEYTCSSETDAQSITYNGSMDSP
VPLYPTDCPPSYEAVMGLRGDSQATLFDPQLHDGSCICERVASIVDVSMDSGSLVLSA
IGDLPGGSSPSEDSCLLELQGSVRSVDYVLFRSIQRSRAGYCLSLDCGLRGPFEESPL
PRRPPRAARSYSCSAPEAPPPLGAPTAARSCHRLEGWPPWVGPCFPELRRRVPRGGGR
PAAAPPTRAPTRRFSDSSGSLTPPGHRPPHPASPPPLLLPRSHSDPGITTSSDTADFR
DLYTKVLEEEAASVSSADTGLCSEACLFRLARCPSPKLLRARSAEKRRPVPTFQKVPL
PSGPAPAHSLGDLKGSWPGRGLVTRFLQISRKAPDPSGTGAHGHKQVPRSLWGRPGRE
SLHLRSCGDLSSSSSLRRLLSGRRLERGTRPHSLSLNGGSRETGL”

mRNA join(32168..32299,32668..32755,33308..33499,33623..33769,

34735..34868,35079..35251,35806..36043,36915..37139,
37540..37703,38073..38189,38284..38931)
/gene=“GBA”
/product=“glucocerebrosidase”

gene 32168..38931

/gene=“GBA”

CDS join(32273..32299,32668..32755,33308..33499,33623..33769,

34735..34868,35079..35251,35806..36043,36915..37139,
37540..37703,38073..38189,38284..38389)
/gene=“GBA”
/codon_start=1
/product=“glucocerebrosidase”
/db_xref=“PID:g2564914”

/translation=“MEFSSPSREECPKPLSRVSIMAGSLTGLLLLQAVSWASGARPCI
PKSFGYSSVVCVCNATYCDSFDPPTFPALGTFSRYESTRSGRRMELSMGPIQANHTGT
GLLLTLQPEQKFQKVKGFGGAMTDAAALNILALSPPAQNLLLKSYFSEEGIGYNIIRV
PMASCDFSIRTYTYADTPDDFQLHNFSLPEEDTKLKIPLIHRALQLAQRPVSLLASPW
TSPTWLKTNGAVNGKGSLKGQPGDIYHQTWARYFVKFLDAYAEHKLQFWAVTAENEPS
AGLLSGYPFQCLGFTPEHQRDFIARDLGPTLANSTHHNVRLLMLDDQRLLLPHWAKVV
LTDPEAAKYVHGLAVHWYLDFLAPAKATLGETHRLFPNTMLFASEACVGSKFWEQSVR
LGSWDRGMQYSHSIITNLLYHVVGWTDWNLALNPEGGPNWVRNFVDSPIIVDITKDTF
YKQPMFYHLGHFSKFIPEGSQRVGLVASQKNDLDAVALMHPDGSAVVVVLNRSSKDVP

LTIKDPAVGFLETISPGYSIHTYLWRRQ”

misc_feature complement(38948..42406)

/note=“metaxin pseudogene”

misc_feature 54895..59543

/note=“glucocerebrosidase pseudogene”

mRNA complement(join(<59671..59885,59978..60132,60598..60674,

60801..60983,61149..61241,62741..62820,62953..63022,
64036..>64116))
/product=“metaxin”

CDS complement(join(59671..59885,59978..60132,60598..60674,

60801..60983,61149..61241,62741..62820,62953..63022,
64036..64116))
/codon_start=1
/product=“metaxin”
/db_xref=“PID:g2564913”

/translation=“MAAPMELFCWSGGWGLPSVDLDSLAVLTYARFTGAPLKVHKISN
PWQSPSGTLPALRTSHGEVISVPHKIITHLRKEKYNADYDLSARQGADTLAFMSLLEE
KLLPVLVHTFWIDTKNYVEVTRKWYAEAMPFPLNFFLPGRMQRQYMERLQLLTGEHRP
EDEEELEKELYREARECLTLLSQRLGSQKFFFGDAPASLDAFVFSYLALLLQAKLPSG
KLQVHLRGLHNLCAYCTHILSLYFPWDGAEVPPQRQTPAGPETEEEPYRRRNQILSVL
AGLAAMVGYALLSGIVSIQRATPARAPGTRTLGMAEEDEEE”

mRNA join(<65492..65570,66961..67167,68050..68306,68409..68511,

69827..69853,70061..70143,70229..70280,70404..70552,
70963..71103,71330..71407,71795..71947,72161..72271,
72361..72468,72754..72913,73251..73369,73508..73560,
74757..74950,75138..>75270)
/gene=“THBS3”
/product=“thrombospondin3”

gene 65492..>75270

/gene=“THBS3”

CDS join(65492..65570,66961..67167,68050..68306,68409..68511,

69827..69853,70061..70143,70229..70280, 70404..70552,
70963..71103,71330..71407,71795..71947,72161..72271,
72361..72468,72754..72913,73251..73369,73508..73560,
74757..74950,75138..>75270)
/gene=“THBS3”
/codon_start=1
/product=“thrombospondin3”
/db_xref=“PID:g2564912”

/translation=“METQELRGALALLLLCFFTSASQDLQVIDLLTVGESRQMVAVAE
KIRTALLTAGDIYLLSTFRLPPKQGGVLFGLYSRQDNTRWLEASVVGKINKVLVRYQR
EDGKVHAVNLQQAGLADGRTHTVLLRLRGPSRPSPALHLYVDCKLGDQHAGLPALAPI
PPAEVDGLEIRTGQKAYLRMQGFVESMKIILGGSMARVGALSECPFQGDESIHSAVTN
ALHSILGEQTKALVTQLTLFNQILVELRDDIRDQVKEMSLIRNTIMECQVCGFHEQRS
HCSPNPCFRGVDCMEVYEYPGYRCGPCPPGLQGNGTHCSDINECAHADPCFPGSSCIN
TMPGFHCEACPRGYKGTQVSGVGIDYARASKQVCNDIDECNDGNNGGCDPNSICTNTV
GSFKCGPCRLGFLGNQSQGCLPARTCHSPAHSPCHIHAHCLFERNGAVSCQCNVGWAG
NGNVCGTDTDIDGYPDQALPCMDNNKHCKQDNCLLTPNSGQEDADNDGVGDQCDDD
AD
GDGIKNVEDNCRLFPNKDQQNSDTDSFGDACDNCPNVPNNDQKDTDGNGEGDACDNDV
DGDGIPNGLDNCPKVPNPLQTDRDEDGVGDACDSCPEMSNPTQTDADSDLVGDVCDTN
EDSDGDGHQDTKDNCPQLPNSSQLDSDNDGLGDECDGDDDNDGIPDYVPPGPDNCRLV
PNPNQKDSDGNGVGDVCEDDFDNDAVVDPLDVCPESAEVTLTDFRAYQTVVLDP”

What is claimed is:

1. An isolated nucleic acid comprising the nucleic acid set forth in the Sequence Listing as SEQ ID NO: 2.

2. The isolated nucleic acid of claim 1 in a vector suitable for expressing the nucleic acid.

3. The vector of claim 2 in a host suitable for expressing the nucleic acid.

4. A polypeptide encoded by the nucleic acid of claim 1.

5. An isolated nucleic acid encoding the polypeptide of claim 4.

6. A purified antibody which specifically binds to the polypeptide of claim 4.

7. An isolated nucleic acid comprising the nucleic acid set forth in the Sequence Listing as SEQ ID NO: 4.

8. The isolated nucleic acid of claim 7 in a vector suitable for expressing the nucleic acid.

9. The vector of claim 8 in a host suitable for expressing the nucleic acid.

10. A polypeptide encoded by the nucleic acid of claim 7.

11. An isolated nucleic acid encoding the polypeptide of claim 10.

12. A purified antibody which specifically binds to the polypeptide of claim 10.

13. An isolated nucleic acid comprising the nucleic acid set forth in the Sequence Listing as SEQ ID NO: 6.

14. The isolated nucleic acid of claim 13 in a vector suitable for expressing the nucleic acid.

15. The vector of claim 14 in a host suitable for expressing the nucleic acid.

16. A polypeptide encoded by the nucleic acid of claim 13.

17. An isolated nucleic acid encoding the polypeptide of claim 16.

18. A purified antibody which specifically binds to the polypeptide of claim 16

19. An isolated double-stranded nucleic acid consisting of 1) a single-stranded DNA which has a molecular size of 6.4 Kb and is derived from humans, and 2) a DNA complementary to the single-stranded DNA, giving the restriction pattern corresponding to the propin1 gene shown in FIG. 1.

20. A single-stranded RNA corresponding to the single-stranded DNA of claim 19.

21. A single-stranded RNA corresponding to the complementary DNA of claim 19.

22. A polypeptide encoded by the isolated double-stranded nucleic acid of claim 19.

23. An isolated double-stranded nucleic acid consisting of 1) a single-stranded DNA which has a molecular size of 8.3 Kb and is derived from humans, and 2) a DNA complementary to the single-stranded DNA, giving the restriction pattern corresponding to the cote1 gene shown in FIG. 1

24. A single-stranded RNA corresponding to the single-stranded DNA of claim 23.

25. A single-stranded RNA corresponding to the complementary DNA of claim 23.

26. A polypeptide encoded by the isolated double-stranded nucleic acid of claim 23.


SEQUENCE LISTING (10 sequences)

SEQ ID NO: 1 - DNA, 75270 bp, Homo sapien
tcccagggtcccgggttgggggggtggagcagcatttcgtcgccgcgggggtgccgggactccggccgcagtgtcgccgccatcacggacttcctgtgggacaagcgcacgggcctcgccgccagaacggtgagcgcgcgggttggctggccgcgcgaaaagatggcgaccgcggggcgggcggggttaggcttgggttgggggcaagggacgggggcgagatttggagaggaggagagttgagacctacgaagcgacggagtagggagatgagggggaaggaggtcggcctgcgttagatgtaccaagaagacctgtaggaagcttgggcggaaccagactagaagggatgatagagtaaggatgggagtggttccagagatgtgaaggagtggaatatcaaagttggactcgggtccaatcaaagctggaggggaggaatacatcaccgtgtcacttggaacatgcaggaagaatttgcctgtaggagttctggattttctgagtaagaaaggaaaaagcaaaagatttcaaacttgagaaaggtaggctagtgatgttttgaggggacagtgctgtgaggccgtttgatggtagtatgtggtgagatggcatttatgtggctatgctgattaggataactatgctgggaaaatcttgtgcttgcagcttggtatgctaccggccccagttacaccaagaatgcatcaaataccaccctcctcaatttggggaggttgggaagaagtgtatacagaatggtggtcagacatcggaatcattaaaccttttgcatacgcgattctcatacacccccaccaagacttatgtccctgggcctgtttcattttattgtgccttcctattctcatcttcctggattgtgacagtgatttttctcttttttccttgaaggaaaaaaaggatttgtgaaccatcccattccaattccattaactggaaggtggccagctgtcagctaagtagggctgatattaattaaaaaccacttactggccaggcgcggtggctcacatctgtaatcccagcactttgggaggccaaggcgggcggatcacctgaggttgggagttcgagaccagcctgaccaacatggagaaaccctgtctctactaaaaatacaaaattagctgggcgtggcggcgcatgcctgtaatcccagctactcgggaggctgaggcaggagaatcgcttgaaccctgcaggcggaggttatggtgagccgagattgcgccattgcactccagcctgggcaacaagagcaaaactccgttttcaaaaaaaaaaaaaaaaacacaagaaacagttgttatctacgtcttaagcacaagagtggaggggccagatgggatgctgactgtattttgtggcctactacattaaaacctctcaagggagagttggccaaagatgtgagagatgattaagttaattcaaggggcccgacccagatccctggagctgatagtatttggcgtttacctggtatatgtagaaacctgggagactggattcctaccctcaggacactgatagtctacttgaggagatgagatgtactcttaatgagtgtacaacatttatcataactgtaaaccaaagctttactctattgacttgtgaggaactcagcatcacacagatctatatcttagatcctgaacggggatacactgtggcagtctccatcggtcgtcatacactttgctctgtagtgaactgtgtcctagacctgaaggtcttcatggagctgtgtttgaagaaaataaggatagaacacttgaactggctgggtgcggtggctcacgcctgtaatcccagcactttgggaggccgaggcaggaggatcacgagtcaggagtttgagaccatcctggccaacatggtgaaaccccgtctctactaaaaatataaaaattagccaggagtggtggtgcgtgcctgtaatcccagctacacaggaggctgaggcaggagagttgcttgaacctgggaagtggaggttgcagtgagccgagattgtgccactgcactccagcctggccacacagcgagactctgtctcaagaataaaataagaacacttgaactgaggggtgggtaatgagggatgggtttgggggctttctagagtttcagccagagttagtaggtggctgtactccaataaggaggaattgtaagtacagaggaagactgaggaagttgtgcctggtaaaaagaggcagaaataagaggggaaagcagcttagcatctttggagcagccagtccttggaggagatttctgaaaactcgaaactttcctatttccgctttcccttgccttccagatgccgcatcctcgaaggtaccactcctcagagcgaggcagccgggggagttaccgtgaacactatcggagccgaaagcataagcgacgaagaagtcgctcctggtcaagtagtagtgaccggacacgacggcgtcggcgagaggacagctaccatgtccgttctcgaaggtgaggcctcaggaagagatactcaaacacagggtcactcgctcattcctttgttcaacaaatacttattggttatatgctaaaaccattgccttaagtagctggctgaaggtaatgtcgggatagataaccacagaagccatttgggctgcagtgttaagtgttagaatacataatgaggaactgaggacgtacccagtgcgtttggttggagtggggcaggatgttaattcagataaaacattgcagaaagcaacatctgtatatgtctggaggacaagtagagtcaggtgggggaagtgtctttgggcagagggaaagcatgtgcaaaggactgggagctacaggtaactaataagctaacagataagacaaagttagatctttgtgtttttaattttttaaaaattttaaaatatcatttatttatttttgagacagagtttcactctgttgcccaggctggagtgcagtggcaagatctcagctcactgcaaactctatggccccaggttcaagttgattcttgtgcctcagcctcccaagttacctgggactacaggcatgcgccatcatgcctggctaattttttttttttttttttgtatttttagtagagacaggatttcaccatgttggcctcaaactcctgacctcaagtgctccacctgccttggcctcccaaagtgctgggattacaggcgtgagccactgtgcccggcccagttttaatattttagtggtatgttcagtggaaagaatgggttaaggatggtttagagcaaggggggatttcattcaggaaagcttgagaaaaaaatggctctttgtttctttgtatcactgcagcgggaaaaaggccccaatagggagtggttctgcagatgccttttcagaggttttggtgacatggcagatgtgcttggttaacctcaacttcttctggtcccttggtcctctccagtgactcaaacaagtgtctgagaacagtttttcttagcattcactctttgtcctcactaaggcaggaccttgtcacagggtatggtagggaatgtgatctgaacagatacacctcattggtatttgttatccaacagcagttatgatgatcgttcgtccgaccggagggtgtatgaccggcgatactgtggcagctacagacgcaacgattatagccgggatcggggagatgcctactatgacacagactatcggcattcctatgaatatcagcgggagaacagcagttaccgcagccagcgcagcagccggaggaagcacagacggcggaggaggcgcagccggacatttagccgctcatcttcggtgagtgccagcccaggcccttcctctccccactcttctgcaggccctctaggactctggtaagtgagcagtatccttgttctcagctgaacattggggcatgaacactgaggtgggcactgagtttgcctactttcttggaagctctccgactcttgaagggccctggatctgctttggagatggatgggcacggagcatttgtgacccccagtgctctccctggcatgttgggcttattgtgttgggagcagcttctccgccccagcggcctccactctttaatggggaccttgcttgttgaactgcctttttcccccaagccctgggctctgtagcccccttggcaggcgggcttgggtgggggcaaggggagatctgtgtctgcccggaagggcattgtgtagagcatggggttgtgggtgacattggcaacaacaccacttctctgctctgcccatgcctctccctgttcctgttttgggtttggggacttgggattatgcgccgctctctcttctcacaccgatccaccttactgcatctgacgtgttcccttccactgtcccccatcatcgtctgtcccccctggctgggcgcctgtgaccggtgacccctctaccacccaccccgcctccctccggcccccggctccgaacactgggcttggttcggaaccccccctgccccccgacagcagcacagcagccggagagccaagagtgtagaggacgacgctgagggccacctcatctaccacgtcggggactggctacaagagcgatgtacaagccaaatcgtaacaatcctatagcctgtaatggtcccatagccatcctaacgtcccaagccaacctgagtcacagcctcttgccttttgctcagtggtgttgtttatctgggtggtttgagttgctgttgagaattgacctctgttgtccactcccagctctcacggcccctgggttaaaggtggtgggaatcacgcaggggttttcttcccctgtgaccatctgtatctgttccccttccttcatctccaccccaggttgctgtccccttttttcttccaactcagctcattccccaccttctctccctccctctccccgaccctgctctctttcatttcagatgaaatcgttagcaccttaggagaggggaccttcggccgagttgtacaatgtgttgaccatcgcaggtaactgtcagtccctccctactatgtggggctaaagagatggttggggttatatggggcttttttgctaattaacctgaggtagaatttcttagtccccctacagccctgttcattttgagacattcttgagaacccagcaaaagcctctcctgccaacttacaggggtggggctcgagttgccctgaagatcattaagaatgtggagaagtacaaggaagcagctcgacttgagatcaacgtgctagagaaaatcaatgagaaagaccctgacaacaagaagtaagcaagcaaggaagtgtgtagggaggctgagagccccaacccctacacgggagagattcctagacctggtttaggcagacagggggagtcctagaccttctcatccattcatcttttgttcatcatcttagagtagtagaacatcagggtaggaaagggggggggcccatgacattccttaatccactccccttgttttcagggaagttagattgctctgtcgtttgaccccagcctagaatggcttctgtagagatataccctggacatagccctaggactgagcatcaacctcatcgaacaatgaattaagcttttataagtagaactatgctgataaggccacagacatttatgcagtaatattttgttcacatacattcacagtccaaaaatagaataaggactttaagccaagatatgaggccagtaggtataaataggagcccagtggtagctagtggtgaatgaatatcaggagttgggctggggtttgggatttaggatggacagtttgatgattccaggatctatactgtttggagcttggcactccacagctcttccagcaaatagtttttgaactatttaaaatgacgcaggaagatattttgaaacttgggctgggcatggtggctcacgcctgtaatcccagcactttggaggccgaggcgggtggatcacaatgtcaggagttcgagaccagcctggccaatatggtgaaaccccgtctgtactaaaaataaaaaaattagccgggcatggtggtggacaactgtagtcccagctacttggaaggctgaggcaggagaatcgcttgaacaggaggcagaggttgcagtgagccaagatcgcgccactgcactccagcctgggtgacagagtgagacgccatctcaaaaacaaaaaagaaaaaaacatggcagatagtaccatttctttcttgggtctggtagaggctactccttagctgaactgaattttggtgttagtactagctggcatggttttacacaagttatgtggaatcaacagctatgaagtacctccttgttggatggctgatgggcagatgggagctcatcagacaatcccccttcccccatctctcttctcagcctctgtgtccagatgtttgactggtttgactaccatggccacatgtgtatctcctttgagcttctgggccttagcaccttcgatttcctcaaagacaacaactacctgccctaccccatccaccaagtgcgccacatggccttccagctgtgccaggctgtcaagtgtgagtggggtgggccgaagtggactctggggcagtccctcccttcattggatctcttctgtcggttgtgcactggtgaagcccctaaacagtcagctgtctgttatctgcagttctttgatttactgtcatcttgaaacgtcttctgacttaactccttgactgatgtctttatcgtcactgattgctcttactctacacctagcctagcagcagctagaagagaaagcctttggaatcaaagcacttaattaccctccccttttcctttctccctttcttgggaaagcatcagtcagacagcaaacataaagagacaaaaatacactccttagggtaaaggcttacatttgtctgggatgagatgttcattcacagcaaaggagatgggaacacagagtatgtagttcagctaaggggcaaggtggggaattcaaagaaatatatcattcctctggagtcatcaaaataatacagtttcacagaattgagttaacataatgccagctagacacatgtcaaagtgtgtacacactacaactcaaagcaaactttttttttttttacatcagttgagctacatatgtatcttacattagaaagagcagagcttcttagaccaggcattccatttagacgtagagcggaaagcagccctagtgattctggacctgtctcctcactggctttgccctaggtaaccaggcctggggtcagctgataccagttagctctggccacctgcaccaagcctgacctggccttctcccctacagtcctccatgataacaagctgacacatacagacctcaagcctgaaaatattctgtttgtgaattcagactatgagctcacctacaacctagagaaggtaagatggatagggtctgcccttggttactgggggcaggcagctgcaccactttgccttctgccgagccctttgttttctcccttttatttcgtcctcccacattttctccctgactggctccaactgggtaaaactaaataggttgaaagggagaaatctcttaagaagacttaaattgggagataacttgtacaggggacttcagataactttccagtagagtgaaagtttttaaggttcttggtttgggctttatttatttatttatttatttatttatttatttatttgagacagagtctcactctgttgcccagactgatgtgcagtggcataatctcggctcactgcaacctctacctcccaggtttaagtgattctcctgcctcagcctctggagtagctggggttacaggcacccgccaccacgcctggctagtttttgtgtttttagtagagatggggttttgccatgttggccaggctggtctcgaacccctaacctcaggtgatctgcctgtctcgggctcccagaatgctgggattacgggtgtgagccacagtgcctggcccggacttcttttttgagatgtgtatttctgtgaggtaggaaagctcggctcctttgactaactggagataatgaagatctcagacctaaagaagctctccctcctttgacccctttgctacatgttacatatttttagagaaactcctttgctacatgttacattttttcagaaaaacccatttgctacatgttacattttttcagaaaaacccatttgctacatgttacatgttttcagaataactatatatctggcacttgagtgtagtcttcagatgcttacagatgtgcacgctgttctataatcatttcttaatccttttgctcccctacttctaaaagtattatgctggatgtggtggaagctgtaaaacaggaatttcccaggcttgtaaggccaggcaggagatcacagtaccttttttaaggctcagaagcaatgtagagaatactggtggaatctcagtctgggtgcagaggttcatcctctttctcctctgctcctagaagcgagatgagcgcagtgtgaagagcacagctgtgcgggtggtagactttggcagtgccacctttgaccatgagcaccatagcaccattgtctccactcgccattaccgagcaccagaagtcatccttggtaagggagggcaaggctgtccaagtgtgtgagatgatgtgagggtggggccacctaagcctcataacaccttttccctcccattctcacccagagttgggctggtcacagccttgtgatgtgtggagtataggctgcatcatctttgaatactatgtgggattcaccctcttccaggtaagtgatgggatgtcttacttgactgcctggcattcttctacctggttcctttgttttctgctgaggacctgcctactcagttctccacattgcctgccttcctggcagctgatccttagcatgcccttttcagtcccatgctcagtttctgtttttgtttcccagacccatgacaacagagagcatctagccatgatggaaaggatcttgggtcctatcccttcccggatgatccgaaagacaaggtgaaccttgagggggcactagttaactcttttccttttctctccacagaattggtctatttcacatcattttcttttttctttgatacctcctctccccccagttactttcagatggggaaataagggaattgtaacaagggtgaccttctgattcctcaacctccccttcccctctagaaagcagaaatatttttaccggggtcgcctggattgggatgagaacacatcagctgggcgctatgttcgtgagaactgcaaaccgctgcgggtgagctgggctcgggataaatagtgcccaccgtccagaagtcacttccttcttagggtggttgccccctggaatgctcttcaacaagccagagggttaggaaaggaggggaggaaagctgaaagaagacatctttggtcaacagaggaaacataagagggagtggttttgcggagggaaggaggttagacagcctaaccttgagacaaccagagatcaaagcaatgtcctggattctttaggtcagacagaaaagaataaactacccttgaagagcttacattttaatgaggaactaaagaagattcatgaagttgacaaggatatacaagtagaaagaactttcaaagattatggagtaattgtgctagagggaaggtaggttgagctataatatcagaaacgttggtcctggtgtggtcgtggttaggtagccttcaaattggttgcaagcagagccttggttctccaagaatgaaaggtagggtcttgaagaaggcagggtttgtaaggcatcctgcctcacctttttcctgccctcctccaccagcggtatctgacctcagaggcagaggaacaccaccagctcttcgatctgattgaaagcatgctagagtatgaaccagctaagcggctgaccttgggtgaagcccttcagcatcctttcttcgcccgccttcgggctgagccgcccaacaagttgtgggactccagtcgggatatcagtcggtgacgatcaggccctgggcccccctgcatcttttatagcagtgggtgtccagtccaggacactggtgcttttttatacaagagaacgagccagagttcactccttcctcctggctctctatatacctgtgaatatgtgaaatagtgtaaatatgaaagaacttgtacctatcacttcaacccctgccttgtacataatactattccatccacacagtttccaccctcacctgccccctcatacggagttggatgggggccgagtgaggtaaccaggtggcatctaccccatgttttataaggaattttgtacagtctttgtgaaataaaataacgtgcttcatttgacccccatccctggagttggaggtttgggaatgctggggtggagggatgaaactattggcaaactttctgagtttgggtatgaagggagtcctccttaccctccaaaatgaagcacagccaggctaccatttatttcccctgtccaccttatcatatgggagggtagtgatgggtggggcagcatttctttcagattaaaacagagaagtgttatgaggtggcacttctcggatgtggaattatgagagttgggaagatctgactcctagagtcattaggccgcggcccagtatagagccagaaactcaggttgaaaacgtgctcaacctggctcctaggggggattgccaggaccggtcagaaggctcccgtcgtccatctcgggagactaggaaggccggattcctacgcgaggcctgctgggaagtgtagttcgttagtggaaggaagtcacatggaagaggggcggtagttggttgtgggcactgggttagaggtatcacgtgggggcactttcgtcttagcttttggacaagacgcaggcgcaaacccacggctgctgcgggggatccttgtggccctttccggtcggtggaaccaatccgtgcaacagagaagcggggcgaactgaggcgagtgaagtggactctgagggctaccgctaccgccactgctgcggcaggggcgtggagggcagagggccgcggaggccgcagttgcaaacatggctcagagcagagacggcggaaacccgttcgccgagcccagcgagcttgacaacccctttcaggtgacttgcgccagtcggcctcttttgggcggtcaggttgattcttcccggttctgtagggtcgggctaacttgtatccccatttgtgacatttgatcctgggaagagccgccacgtggggtgacagtgactccagaccagtgagcactctggggggcgggccctgccccttaatgggctggtgctggcagtgttggatgatggatttgaggtaaatgttgtcccagtcctgggacaacggcgtggccccgaaggtgcagtacttggagccacaggcagtttgggaatggtccctgggaattcccctaggtggcactgggtgccagctgagacccgggtctctgccctcaggacccagctgtgatccagcaccgacccagccggcagtatgccacgcgtgacgtctacaacccttttgagacccgggaggtgagctactggagagcataggagttcaaggaagggaagggtttgccagtgagacaagttagcttgggtacaggggacttttctgcatgcacaggaaggaagaggcactgtgatcttagttccctgagagaggctagtcagccatgggatgcctcatccttctagggccaccagggccaccaacccaaatggtttgagctctaaaagtcagtcctgggaagatctggaggctggcaaggccttaaccaatgttctgtatgggaaggctttcttgaagaaaataaaggttggggctggttggagtttaggttaggtgtgcagtttgaaagcaaggaatgaggaggactacacctgtttgtcccactggattagaagatggtgagttgtaagaatttaaggggttttcatggtacttaaaacttaaaaaaaaaagccaagcatggtgtgcatgcctgtaatcccacttgggaggctgaggtgggaggattgcttgagcccaggaatttgatggagaccatcctgggcaacagcgagacccccatctcttgagacgttttaaaaattggctaggtgtagtggcacatgcctgtagtccttgctacttgggaggctgggatggaaagactgcttgggcccaggagtttgaggcttcgatgagtgatgattgcaccactgcactccagcctgggtggcagagagacaaccccacactccatccccagaagaaaagagaaaaaaaaaacacagttaagtggggagatatggtaaatgagtgatttgagtcctcactaaggaattggatatgagggatgataaggctgctacaggtcatcatagagttctctgacagaaattcaacagaagaactagcttcctaacaaatgggggaaaggctgcttccttacatcatgagttctgggtctaaaggtattcaagcagactatagattcctgtgttatgagggactagatgatcactaaggtcttttcctcaacagccaccaccagcctatgagcctccagcccctgccccattgcctccaccctcagctccctccttgcagccctcgagaaagctcagccccacagaacctaagaactatggctcatacagcactcaggtacaggaggtgtgcaggtgagggctggggagaagggccagtcctggggcccagggctcacatctccgtctgctcacactgggcaggcctcagctgcagcagccacagctgagctgctgaagaaacaggaggagctcaaccggaaggcagaggagttggaccgaagggagcgagagctgcagcatgctgccctggggggcacagctagtaagtaatagagtggggaagagcatctgagagtttgggaggagcaaaaacaggtcttaagggcctgggctcggctgggtgcggtggctcacacctataatcccagcactttgggaggctgaggtgggcggatcacgaggtcacgaggtcaagaaatcgagaccatcctagccaacatggtgaaaccctgtgtctactaaaaatacacacacacaaaaattagctgggcatggtggcgcgcacctgtagtcccagctactcgggaggctgaggcagggagaatcgcttgaacccgggaggtggaggttgcagtgatccgaaatacgcgccactgcactccagcctggtgacagaaatgagactccatctcaaaaaaaaaaaaacacaaacctgggctctggagtcagactgctgagtttgaattttagagatactgcttactaaccatgagcttttagggaagttacttaatcttgaatgcctcagtttccctatttataaaatgaaaaccacgtttgcatccgtttgacagggttgttgtgaaggttaaacaaaatatatgtgaagtacttggtacagtgcttagatgttttaaataataataaacagtattaatttttccactctcacttgtctccttggaccccaaattggagataaaagagaggatccagggctgggtatggtgcctcacgcctgtaattccaacactttgggaggccgaggcaggcaggttgcttgagttcaggagttagagaccaacctggacaacatagtgagatcctgcctccaaaacattaatgaaaaaaattagcagggcatgatgtacctgtctgtagtcctagctcctcaagaagctgaggtggacggccaggcgccggggctcatgcctgtaatcccagcactctgggaggccaaggcgggtggatcacctgaggtcaggagttcaggaccagccaacatggtgaaaccctgtctctactaaaaatacaaaaattagctgggcgtggtggcatgtgcccataatcccagctactcaggagactgaggcaggagaattacttgaacccgggaggcggagattgcactgagccaagatcacaccactgcactccaacctgggcaacaagagcgaaactctatctcaggagaaaaaaaaaaaaaaaaaaaagctgaggtgggagggttgcttgagcccaggaggttgaggctacagtgaaccatgatcataccactaccttccagcctgaacaacagagaccctatctcaaaaaaaaaaaaaaaaaaaaaagagaggatccagggatggagagaagggggagtgattcctttgttggtctctgttttacttctggggtccacctgttttttggcgccttacagctcgacagaacaattggccccctctaccttctttttgtccagttcagccctgctttttccaggacatctccatggagatcccccaagaatttcagaagactgtatccaccatgtactacctctggatgtgtgagtagtgagaagccttttggaggaagttacaggtagatctcttaactgccctggggtccgctcaccaagaaccaaacacttcacctctatttagaactcaccagcctgctagcaaatgttcttgccctctccccactttttattgtccatatgcaggaatatattttgaattctttagatgtctttgggctgggtgcagtggtatacgcctgtaatcccagcactttgggaagctgaggtgggtggataacctgaggtcagaagtttgagactagcctgatcaacatggagaaaccccatctctactaaaaatacaaaattacctgggcgtggtggcacatgcctgtaatcctagctactcaggaggctgaagcaggagaatcacttgaacccgggaagtggaggttgcaatgagccaagatcatgccattgcactccagcctgggcaacaagagcaaaactccatctcaaaaaaaaaaaaaaaaaaaaaaaaaaaaaaaaaaaggccgggcacggtggctcacacctgtaatcccagcactttgggaggcccaggtgggcagaacatgaggttaggagatcaacaccatcctggctaacacggtgaaaccccgtctctactacaaatacaaaaaattagccgggcgtggtggcgggtgcctgtagtcccagctacttaggaggctgaggtaggagaatgggcgtgaacccaggaggcagagcttgcagtgagccgagatcctgccactgcactccagcctgggcgacagagcgagattccatctcaaaaaaaaaaaaagaaaaaaaaaaattagctgggcgtggtggcgggcgcctgtagtcccagctactctggaggctgaggcaggagaatggcgtgaaccggggaggcggagcttgcagtaagctgagattgcgccactgcactccagcctgcgcgacagagccagactccgtctcaaaaaaaaaaatgtctttgcaaggggatcacacattattgacatttgctttctccatctcccctgttgaggattccatgaaggcagcagctgccttcatttccttactctgccatgtttggtgaatattataggatgagcatagatgggaaggagccttcattgcagtccagaagggctcctcatcctgtccctctgccccttaggcagcacgctggctcttctcctgaacttcctcgcctgcctggccagcttctgtgtggaaaccaacaatggcgcaggctttgggctttctatcctctgggtcctccttttcactccctgctcctttgtctgctggtaccgccccatgtataaggctttccggtaagtgtgttagtggtgggagagtgatggagacctgggatgggccccacgtctgcccatccttcagctctaattcttctcccaccctccccatttttttcctctttgtaggagtgacagttcattcaatttcttcgctttcttcttcaatttcttcgaccaggatgtgctctttgtcctccaggccattggtatcccaggttggggattcaggtttgtgaggctgttatccaccctcacctttccctctagatccagccagcactgggtgctggataggagttgttcagaaaaaggaaatgtggttttaatccttgggaggtactagtttaatgagatacaagacatacttccaggatagagcgcagacagcactcttgacacatatagattgaagggaagaatgctgcatttggccaatctaaggtggcttcctggaggaggcatagaaccatggttgaaaggaggaagaagaatccccagatgagtgcctggaagagcttggagagctcagggctaatggttcagaaactggaattaaactatgaagagattagatacagtttgggattgtggtgcttggaatgctgctcgattgacagggagccactgctgatgggaagggtaggaacaggggatgctggtgacataaccagtggagaagctgaggagcccctcttcactggtacatccttccctttacagtggctggatctctgctctggtggtgccgaagggcaacacagcagtatccgtgctcatgctgctggtcgccctgctcttcactggcattgctgtgctaggaattgtcatgctgaaacgggtgagggctgtgtcgaaggtggggccgggatggtgagatcatgggtccccaggggcgtgggtggaacattcaggagcaactggcacaggtcaggctgctgggttgttctcagctaatggacctctggggtgtgtgtttctgtgtgtgagtgtgtgtgctgggcagcaggctgctgagtggtagtgatgctgttaggctgggggtggggaaccagtggctggaatgggcggtaatgtctttgtcctctacttgcagatccactccttataccgccgcacaggtgccagctttcagaaggcccagcaagaatttgctgctggtgtcttctccaaccctgcggtgcgaacccgagctgccaatgcagccgctggggctgctgaaaatgccttccgggccccgtgacccctgactgggatgccctggccctgctacttgagggagctgacttagctcccgtccctaaggtctctgggacttggagagacatcactaactgatggctcctccgtagtgctcccaatcctatggccatgactgctgaacctgacaggcgtgtggggagttcactgtgacctagtccccccatcaggccacactgctgccacctctcacacgccccaacccagcttccctctgctgtgccacggctgttgcttcggttatttaaataaaaagaaagaggaactggaactgacatccccgtttcctgaatcttcattgggaattaggccttatgaaagagaaaggagagtgtggagtgaggtgggaaggtcggacccggctttagtgtaaactgggagagagatgaggggcggggcggtggagcccgagtggctggcgcaggaaggaggtgggaagtccacggaaacgcgaaacccggagacgccagggagccctgctccccacccctctccattaatgacggggaagagccaccgcctctgccgggaacgccaaggaatacgcgggcctggagcctgaaaagctggatggggctcaagtggaggcccaaaggatcactagcagcctagccagggtccagagcgaggcagggactggaggagcgcttatccgactgcctcgccctgccgcgggatcccccaaccccgacagggtctcagtcccgaactacaactcccggggtgcaccgcgccggccctcgccgccatgcccctccttcccgcaccatccccattcccatcccccttctctagtccccgacctgcggcagccggagctcggggagcggagcgtggtggggaggggagcgggacaggcgacacaggagacagcggcgccgcggcctctccccaccaggcggccccggatcctactggacgccctgagggcacaccgaccgcgcctctagagtcaccccacgccgacccctcccctcttctctagacttatttccatccttcccgcttttaccctccccacccgtccctgggctccaggccgccgccccctcctcactcctggaccggcccttctcggtgcccctcttccctagggagatgcgatgagccggtgcccccgcgtcctcatcgtcgccccgggcacggtgcccgtccagtgcccgtggtggggagggagcactccgcggtccctccgtgacgcccctcgcttggccccccccacagctggcgtccctcggccatgccccaggggacccagccagggggtgggctctagagcgagtggggtggagaggagaaaggacggggccttgggggcctctgagatgctcccaagtgccagggagggccgagcgaggcgcaggcaaccgggcagcaggcatgatgccctcgcctagtgactccagccgctcgctgaccagccggcccagcaccaggggccttacccacctccgcctccaccgaccctggctgcaggccctgcttacgctggggctggtccaagtgctcctgggcatcctggtggtcaccttcagcatggtggcctcttccgtcaccaccaccgagagcatcaagaggtcctgcccgtcttgggctgggttctcggtgagttgggtgcacagttgttgggtgggggaggctcctcgggccccaccctccacaccagctgcaagtccacatgcttgcctctcctccctctcctggtcctctgcctccttacagggctgggtgcatcctgtggtgaggggctcactcaggagcctccccctgccaggctgagcctgtgcccactctgtccccagctggcgttctccggggtggttggcattgtgtcctggaagcggccattcactctagtggtaggtgccagggtccagtgcccactgggaggcaggtgcccagcacgcaaggggaagcccatttatgacctcaagaagggaactggctccccagttggtgccagccgggtgggcacgaagttctgtgaaggagggggactctgtccgtggcagaggagtactcatgagggtcccagccctgagtctgcaccctgttttccccagatctccttcttctccttgctttcggtgctctgtgtcatgcttagcatggctggctctgttctctcctgtaagaatgctcaactggcccgagacttccaacagtgctctctggtgagatttgaggagggagagctggaaagaactggctgggggaggtgtgcaggacacctcagtttgtgctgactcaggctgcctcaccctccctgctccactcaggaaggaaaggtctgtgtgtgctgtccctctgttcccctcctccggccctgtccagagtcggggcaggaactgaaagttgcccctaactccacctgtgatgaagcccgaggggccctcaaggtgagcttgcaccctgcaaacatcctcctggttctcactcttgcctcccctgctggggatctcacaggcccataatgtgtggaacttagccgtctcctgactcctctcctcctttcccttccccagaacctgctcttcagcgtctgtgggctcaccatttgtgccgctataatctgtacactctctgctattgtctgctgcatccaaatcttctccctggacctcgtgcatacggtgagaagggagcaggggccagggcacgcaggtatggtgggggcaggggtgtgtgtgctgagacttgcctgagggaacaatggctacagatctggcttttgagcaccaggggctatgggttatctattatcctcatctattggaggaatggatgtttagtggggaggatgagaaggggagatggcagggagtgagttaagtatgtgatgctggtctgggaccaggagagggtgctttagagaatgggactggatggtggggtagagtcaagaaggtcctatgctgggcacggtggctcattcctgtaatcccagcactttgagaggcccaggcaggcggatcacctgaggccaggagttcaagacccacctggccaacatggcgaaaccctgtctctattaaaaatacaaaaatcgggccgggcgcagtggctcgtgcctataatcccagcactttgggaggctgaggagggcagatcacctgagatcaggagatcgagactatcctggctaacatggtgaaaccccatctctactaaacatacaaaaaattagccaggcgtggtggcgggcgcctgtggtcccagctactcgggaggctgaggcaggagaatggcatgaacctgggaggcagagcttgcagtgagctgagatagtgccactgcactccagcctgggcgacagagcaagactccatctcaaaaaacaaacaaacgaaatacaaaaattagccaagtgtggtggcgggtgcctgtaatcccagctacttgggagtctgaggcaggaaaattgcttgaaccggggagtcagaggctgcagtgagctgagatggtgccactgcattccagcctgggcgacaagagcacagactccgtctcaaaaaaaaaaaaaaaaaaaaaaaaaaaatatatatatatatatatatatatatatatatacacacaaaattacaaaaattaggctgggctccgtggctcatgcctgtaatcccagcactttgggaggccaaggcaggaggatcaccagatatcaggagtttgacaccagcctggccaacatggcgaaaccctatctctactaaaaatacaaaaattatccgggtgtggtggcgggtgcctgtaatcccagctactcggaagactgaggctggagaatcgcttgaacctgggaggcagaggttgcagtgagctgagatgtagccattgtactccagcctgggcgacaagagtgaaacttcgtctcgaaataataataataataataattagctgggcatggttgcacacccttataattcctgctactcaggaggatgaggcatgggaattgcttgatcttgggaggtgggggttgcagtgagctgagatcgcgccactgcactccagcaacagagtcagactctgtctcaaaaaaaaaaagaaggtcctagatggaggtgaggctaaagggtggacattcctgtgaagacacagaatgtgtggagcttctggatggaggtggggccataaagaggaatttgtgggtgggcagggccagaggctagaggtacagggtgacggtggggttaaggagagctgattttgggtgagggaggggccagaataagagcttctctcaggggatagggccagaaaaaataatgtagatgagagaggagtttggggcccaggaggagctgtattcccagaatccagacttgctgacctgctcgcttccccagcagctggcccctgagcggtcagtctcaggcccactgggacctctgggctgcacgtccccgcccccagcccctctcctacacaccatgctggacctggaggaatttgtcccgcctgtgcccccaccgccctactatcccccagagtatacctgcagctcagaaacagatgcacagaggtaaggcctggtggggtcttgctgggggagctaaggaaggggactggggctgggcctggattgctggagagagggatctttgtggagggggaattatttctcctacattgaccctcttcctcatctgccagcatcacgtacaatggctccatggacagcccagtgcccttgtaccctaccgattgccccccttcttatgaggcagtcatgggactacgaggagacagccaggtgagagcacagcacggcttggggcgggctggggagccgggttgtagcctggaaagctgaacaggctgtagcctctggataaagatagtaacagtggtcaaggtctttacagcaccagcatgcccactgttgcctttgatcttccctagagcctggtgaggtgagtgggttggaagctatcatttctgccatacagacgtggaagctgaggctgcacaattaagtgacttgtacaaagggacaaggctggtctggaatctaagtctccagagctctgtctagcacatgggggtgttcagtgtgtagggctgaacccttgaccctgtgtcttctgcaggccactctctttgaccctcagcttcacgatggctcgtgcatctgtgaacgagtggcctccattgtagacggtgagcagggcgtaatgaggggtggacaagggcggggctgccagggatagctggggtgggtagagacaatagaaggggaaaacaaggcggagttggtggtttggggacataggagggctgtggcaacttggaaccctggacttatttttctcctctgagataaagctggaggcacagtggcccctagaggctgggcttgggagaagaggaactgcctgggcagggctaggccaggccaggctggtgctacacagcgccccctgccgcccacagtgtccatggacagcgggtctctggtgctgtcagccattggtgacctccctgggggctctagcccgtcggaggactcgtgcctgctggagctgcagggctccgtgcgctccgtggactacgttctctttcgctccatccagcgcagccgtgccggctactgcctcagcctggactgtggcctgcggggccccttcgaggaaagccccctgccacggcgccccccacgggctgcccgctcctattcctgctctgcccctgaagctccacccccactgggtgcccccacagctgcccgcagctgccaccggttggagggctggccgccctgggtgggaccctgcttccccgagctgaggcggcgggtcccccggggagggggccgcccagccgcagccccgcccacccgagccccgactcgtcgcttcagcgatagctcaggttccctcaccccaccggggcaccggcctcctcatccggcatccccaccaccgctgctgctgccacggtcccacagcgacccaggcatcacgacctccagtgacactggtgagccccctccccgactgcccaggctcaggagagggtaggcactgggagttaggtggccagtgatgcccaccaggattggggcacagttgaggtgggtgaggaggaagaaagggtgagttcactcctctggtagacattctcaggtactcacctcctagggacagaacatccatagcttagcagctaaaaaaggagaatttttttttttttttgagacggagtctctggctctgtcgcccaggctggagtgcagtggcatgatcttggctcactgcaacccgtttcccgggttcaagtgactctctcctgcttcagcctgccgagtagctgggactacaggtgtgcgccaccatgaccggctaatatttttttttttttttttttttgagacggtgtctcgctctgtcacccaggctggagtgcagccgcgcgatctgggctcactgcaactccgcctcccgggttcacgccattctcccccgggttcacgccattctccttcctcagcctcccgagtagtgagtagctgggactacagatgcctgccaccacgcccggctattttttgtatttttagtagagatggggtttcaccgtgttatccaggatggtctcaatctcctgacctcgtgatctgcccgcctcggcctcccaaagtgctgggattacaggcatgagtcaccgtgcccgggcaatttttgtattttttagtagagacaggtttcacccttttggccaggctggtcttgaactcctgacctcaagtgatccacccgcctcggcctcccaaaaggatttattttttgaaaccagttccacagctctcagcttggtccacttatctgtcctccccaagcttcagctgtcacttgttaacatgtataataatagtacttcacgccgggcacggtggcttgcacctgtaatcccagcactgtgggaagctgaggtgggtggatcacctaggtcgggagttcgagaccagcctggctaacatggtgaaaccctatctctactaaaaatacaaaaattagccaggtgtggtggagcgcgcctgtaattccagctactaacgagagaggctaaggcaggagaatcgcttgaacctggaaagcaggggttgccgtgagccaagatcatgccactgcactccagcctgggtgacagagacacactccatctcaaaaacacaaacaaacaaacaaaaaacatgtataataacagtacttcagccataggcattgtactcgaagatgctgagaaagaacagtggcgggcaaggctgttcacacctataatcccagcactttggaggccaaggcaggtggatcacctgaggtcaggagttcaagaccagcctagccaacatggtgaaacccccatctctactaaaaattgaaaaattagctgggcctggtggtggacgcctgtaatcccagctactagggaggctgaggcaggagaatcgcttgaacctgggaggcggaggttgcagtgagctgagatcgtaccactgcactccggcctgggcaacacagtgagactccatctcaaaaaaataagaaaggagatagtactggggaacgctcagcactgtgcgccaggtgctgaacaacaccactgcagtccttgttgtggtggattgtaccatctagttgctggctaatatggacagagatgctggccctttgattggggatggagcgtgggagctgtgaaagctcctctgggcttgagttcccacaggagggtgggcgtgtccacagaacacttccactcactccctgtctccctttctctcttctccccagctgacttcagggacctttataccaaagtgcttgaggaagaagctgcttctgtttcctctgcagatacaggtcaggcatgtggtttgcgccccagggatggggattgggcatggctgcccagccccctctccaccctacaataccattctcttatctctgtctctctgcagggctctgctctgaagcctgcctcttccgcctagcccgctgcccttcccccaagttgctacgtgcccggtcagccgagaaacggcgccctgtgcccaccttccaaaaagttcccctgccctcgggccctgcacctgcccactccctgggggacctaaagggcagctggccaggtcggggcctggtcactcgtttcctccagatatccaggaaagccccagaccccagtgggactggagctcatggacataagcaggtaggaattcggggagccaggaaagatgtttgggaaagcgtggagcttcagattgagccttattgatgatgccctttcttgtgtccctgtccaggtgccccggagcctgtggggccggcctggccgagagagcctccaccttcgcagctgcggagatctgagctctagctcttccctgcggcgtctcctgtctggccgcaggctggagcgtggtacccgcccccacagcctcagcctcaacgggggcagccgggagactgggctctgacctaggcttcttgtcacactgaacacatccagccacaggcaccagctggttgggaccagcagcccccagcatcctcttgcactggctggcacaaaaagaaacctgctgtataccccccaaagtgtccctttccctcctacctctggggtctcttgctgcttgcctctgctgctctggtctgggagagcttctgtcctgtgctgcatgggtatttagactgtgggggagatgccccttcttatagcactggaggaggaaaacaaattcttgtccccctcagaatgagagtggctctttctgatttgcaagggcactatggtcagggcaaaggcatggcccaggtgtttaagtacagggtgacgtgtgcctatgcaatggggtggtaaggcaggcacgaagagtccaaaaaatctaggtggcctctcagctctgccacctctagctgcatgaccttgggcaagctatgtaaccccaattgcctgctccattaaagactgtgaaggtagaatgtttgtaaagctcttaacagtatgtaagccttcaataaatttcagttttccccttgttttcttgatcattctctgtcaccagtgaaatttgttctagtgtctctcatatttaagaaaactctttcaggactgggtatggtggctcacacctataatcctagcactttgggaggccgaagcaagaggatcgcctgagcctaggaattcaagaccagcctgggcaacatagtgagaccctgtctctacaaaaaacaaaaaattagccaggcatggtgggacacgcctgtagtcccaactactcaggtggctaaggtgagaggatcacttgagcttgggaagtccaggctgcagtaagctgtgattgagccactgcactacagcctgggcaacagagcaagaccatgtctcaaaaaaaaaaaaaaaagaaaaaagaaactttcaagacactctttccaaccactaattgtaactctgctcctccttttcacagcaataggttttctttttcttccctccactgttaaacatccattctctcctcacccacccccatcagactccttcccctatctttccacagccactgctctgaccaaactttccagtgaccacagtggtgtcagacccagtgaccatttctctgcctgcatctcacttgacctcgaggcagcaattaatacccataatcagcatcttcttgaatttgtccctttgaaaagggaaatattggctcttctactttgtcctgctgaactgcttaacattggagggccccagggccctcacctaagccctctttcctacctccactctttctataggtggccctactactaaagtccatggctttaaataccatctttctatgtgttaatccatgactccagccttgacctcccatgagcgccatccaactcagcatgtctgcttggatgtctaatgggcatttcagattcaacatggccacaactgaactcttgattcccaccccagcaccggttatttttccactgttcccatctcaatggcacctccattacccatttgcacattccaaaagctcaggaaccatggtgacttcttttcccatatccaacacaaccaatcctatcctgaattcatccacatcccaccacctccccagctacctagctccagccatcctctctccacaacctctgaatcagtctttcacttttcccagcaatccattctccactcagcaaaatgatgataaagcacgtcacatcaaggctctgcctcaatttaatggcttcccattgtatttagaatcatctccaaactcccagagactatggtcgagctacaatctggcccaccttctgttccagccaaatttcctcacagcacaaggacgtttgcacctgctgttttgccaagcatgaaacccttggcccctatatctggtgctatcacctaatatcaggttttagctccattctcaccatttcagtgagcacccaatccccatcgcagtcattctatcacatagccatgtttttttttgtttgtttgtttcattttgtctttttttgagacagggtcttgctttgttacccaggctggagtgcagtggtgtgatttgggctcactgcaaccttccacctcctgggtcaagcaattctcctgcctcagcctcccgagtagctgggattacaggcgcccgtccccatgcccgcccagctaaattttgtatttttagaagagatagggtttcaccatgttggccaggcgggtctcaaactcctgacctcaagtaatccgcctgcctcggtctcccaaagtgctgggattacaggtgtgactcaccgcgcctggccacatacccatggtttcagcatgtatcactatctaaaattattatttttgtttatatatctgtgtcgtcccatagaaggttaaggtcccaagatcagaaacttgctcattgcagtgggtctaacactcagtaggtcctcaacaaacattcgttaagatactaaagtggcagggtggggccctgtaaacagcttcaggagcttcaggaccctgtgcttgtaggggcaacgtggtgccctccaaggaagacagggaggtgggaggagcactgcccagagatggcgtcaggctgcaagacttcttgaataattcagcatcataacaacccagcctcaggaagggatagggcacggccaggacgaaacattaggaggcgatggacaatgggattcccacggggcagcttctgcgcactggacgttccctaacctgaggctctctaaagaggaaggttaggaatcctctgagcttcggtgggctggactcactgtgggaattcaatcgcccccatccaccaacagtgtgctggcgggaaaacgccgacacgcatgcgtagttctcgcgccggctcctctctctctctctctctctctcgctcgctctctcgctctctcgctctctctcgctcgctctctcgctctcgctctctctctctctccggctcgccagcgacacttgttcgttcaacttgaccaatgagacttgaggaagggctctgagtcccgcctctgcatgagtgaccgtctcttttccaatccaggtccgcccgactccccagggctgcttttctcgcggctgcggtgatcggtcgggctgcatcctgccttcagagtcttactgcgcggggccccagtctccagtcccgcccaggcgcctttgcaggctgcggtgggatttcgttttgcctccggttggggctgctgtttctcttcgccgacggtaggcgtaatgaatatttcgacctttggatcttagctgtcccctccctgcgttcgcacttaacctttttcaccattattattattattgttattattattattttttgagggagtctcgccctgtcgcccaggctggagtgtaatggcgccttcttggctcactgcaacctccgcctcccgggttcaggcgattctccgacctcagcctcccaagtacgtgggattacaggcacccgccaccacgcacggctaattttttgtatcttttagtagagacggggtttcaccatgttggtcaggctggtctccaattcctgacctcgtgatccgcccgcctcggcctgccaaacagctgtgattataggcgtgagccaccgcgcccggccaaccatcattattatttttaacggtaaggatggtcagattttactaatgaagaagagattataaaatcttcaagtctttatatccacttgctttttgaggggtggagtgggaagaaggttatgtaattcatacgttcttcagagatgtgacaaacattcacggagccggacgacgtcgggttggattcgcactggagctgcagatgggtgccaggatggactggtccctaccctccgcttgaacctaggaggcggaggttgcagtgaaccgagatcgtgccactgcactccagcctgggtgacagagatactccgtctcaaaaaaaaaaacaaaacaaaaaacaagcggactgggcgcagtgcctcaccctgtaatcccagcactttgcaaagccaaggcgggaggatcctttgagtttaggagtttgagaccaacctgcgcaacacagtaagaccccgtctctacaaaaaatacagaaattagccaggtgtggtggtgtgcgcctatagtcccagctattctggaggctgaggtgggaggattgcttattctggaggcagaggttgcactgagccgaaatcaagctactacactccatccagggcaacatacggagaccctgtctcaaacaaacaaacaaaaaattgctcagtacctggccaaaaaagaagaggctcactatgcagaggggaagtggaaggagatgtttggacttctaaactcaatagagcaggagaggcaaatgtagaatgtgctcaggaaatatctgtgagatgaatgaacttgagggaagtaaggtactagatattacctgccctacccagaacaaatcctgtgcaatgtttccttgaaaagtgagaagtctggaaggggtggctactgacatagtgaagcaactagttcaattctacaacttgacagctaccctgtgccaggctatctacgaggatacttagaatgcataagacattccttcaaggaactccaggaacagaggcctgacatgttgcaatgtttagtgtcaagcagtgtactagagacacattatcacactcaaacctcacaacaattctgtgaggtaggagttatcactccccttttatagatgaaacagaggcttagagtgattgatttattgaaagtcaaacagccagtaaatggtgtagccaggattccaaacttgctgtctcactgagactgtacttaattactggagggaccgggtgtggtggctcattgctataatcccaacaccttgggaggctgaggctggtggatcacctgaggtcaggggttcgagaccagcctggccaacatggtgaaaccccatctctactaaaaatacaaaaattagctgggcatggtggtgggctcctgtaatcccagctactcaggaggctgaggcagggcaattgcttgagccgagatcacactgcactccagcctgggcaacagggcaagactctgtctcaaaaccaaaaaaaaaaaaattactggaggaacctagaagaagaaatgatcaattttgcttggagtgtatctagaaagacttcactgagatcatttaaagaacaaaaaggatggctggggtccaggcagtggctcatgcctgtaatcccagcactttcggataccaaggcagcagatcacctgaggtccagagtttcagaccagcctggccaacatagtgaaaccccatctctactaaaaataaaaaaattagctgagcatgttggagggcacctgtaatcccagctacttgggaggctgaggcaggagaatcactcgaacccaggaggtggaggttgcagtgagccaagatcacgccactgcactccagcctgggcaacagagtgagactctgtctcaaaaaacaacaacaacaaaaaatacaaacaagagacaagtagttcccaggtgcctaccaagtggtcaggcactgcacttacctcactgactgcagtaaccaccctttgaggttgtggcattgctccattttccaggcaaggaaatgggctgagagctgggattagtcaggtcatgactgtgtgtgccactcccgctaaatctcatttgatgtggttcatgaggccacaccatggacagcttcctccttgtgtccactgaggatatggctttgtacaacactttggtttttgaacgactttacaaacctccctgtcttgtgaggaaggaagaacagttattaccatctgcatctgatgatgaaacaagggacgctgcagaggagccgcactgaccactccctccctccagtcctgtcatcccactgccagtgtcccaccctcttgtgccctgcacttcactggctaataacccccctcactttttcctctgtgaagccatcctggataattccccacccacgaatggtccctcctcatctcagagagctctccatgcacacctgttaccgtttctgtctttatctgtaaatatctgtgtgtctgacttccatgcctcacacacctctatagggcaaagactgtcttaaacatcttggtagtgtcagtattttgcacagtgaagtttttttttttaaattatatcagctttatttgtacctttttgacatttctatcaaaaaagaagtgtgcctgctgtggttcccatcctctgggatttaggagcctctaccccattctccatgcaaatctgtgttctaggctcttcctaaagttgtcacccatacatgccctccagagttttatagggcatataatctgtaacagatgagaggaagccaattgccctttagaaatatggctgtgattgcctcacttcctgtgtcatgtgacgctcctagtcatcacatgacccatccacatcgggaagccggaattacttgcagggctaacctagtgcctatagctaaggcaggtacctgcatccttgtttttgtttagtggatcctctatccttcagagactctggaacccctgtggtcttctcttcatctaatgaccctgaggggatggagttttcaagtccttccagagaggtaagagagagagctcccaatcagcattgtcacagtgcttctggaatcctggcactggaatttaatgaatgacagactctctttgaatccagggccatcatggctctttgagcaaggcacagatggagggaggggtcgaagttgaaatgggtgggaagagtggtggggagcatcctgatttggggtgggcagagagttgtcatcagaagggttgcagggagagctgcacccaggtttctgtgggccttgtcctaatgaatgtgggagaccgggccatgggcacccaaaggcagctaagccctgcccaggagagtagttgaggggtggagaggggcttgcttttcagtcattcctcattctgtcctcaggaatgtcccaagcctttgagtagggtaagcatcatggctggcagcctcacaggattgcttctacttcaggcagtgtcgtgggcatcaggtgagtgagtcaaggcagtggggaggtagcacagagcctcccttctgcctcatagtcctttggtagccttccagtaagctggtggtagacttttagtaggtgctcaataaatccttttgagtgactgagaccaactttggggtgaggattttgttttttttcttttgaaacagagtcttactctgttgcctgggctggagtgcagtggtgcaattttggctcattccaacctctgcctcccagattcaagcgattctcttgcttcagcttcccaggtagctgggattacaggcggccaccactacgcccagctaatttttgtatttttagtagagacggggtttcaccatgctggcaaggcaggtctcaaactcctcacctcaggtgatccgcccacctcggcctcctaaagtgctaggattacaggtgtgagcccctgcgcccggccaaggggtgaggaattttgaaaccgtgttcagtctctcctagcagatgtgtccattctccatgtcttcatcagacctcactctgcttgtactccctccctcccaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggtaaccattacacccctcaccccctgggccaggctgggtcctcctagaggtaaatggtgtcagtgatcaccatggagtttcccgctgggtactgatacccttattccctgtggatgtcctcaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggtgaggaggaaggggacaagatgacatagagccattgaaacttttcgtttttcttttctttttttaaaatttttttgaggcagaatctcactctgcccattctgtcggcgagacaggagtgcagtggtgtgatctcccctcacagcaacctctgcctcccaggctatagtgattctcctgcctcagcctcctgagtagctggaattataggcgtgcgccactaccacctggctaatttttgtatttttagtagagacagggtttcatcatgttgaccaggctagtcttaaactcctgacctcaaatgatatacctgccttggcctcccgaagtgctggaattacaagtgtgagccaccgagcccagcagacacttttcttttttctttttttttttttgagacagagtctcgcactgtcacccaggctggagtgcagtggcacaatctcagctcactgcaacctccacctcccgggttcaggtgattctcctgtctcagcctctcgagtacctgggattacaggtgcctgccaccacgcccggctaattttttgtatttttagtagagacagggtttcactatgttggccaggatgattgcgaactcctgacctcgtgatctgcccacatcggcctcccaaagtgctgggattacatgcgtgagccactgacacttttctttgccctttctttggaccctgacttctgcccatccctgacatttggttcctgttttaatgccctgtgaaataagatttcgccgcctatcatctgctaactgctacggactcaggctcagaaaggcctgcgcttcacccaggtgccagcctccacaggttccaacccaggagcccaagttccctttggccctgactcagacactattaggactggcaagtgataagcagagtcccatactctcctattgactcggactaccatatcttgatcatccttttctgtaggaatcggatataacatcatccgggtacccatggccagctgtgacttctccatccgcacctacacctatgcagacacccctgatgatttccagttgcacaacttcagcctcccagaggaagataccaagctcaaggtaggcattctagctttttcaggccctgagggccctgatgtctgggggttgagaaactgtagggtaggtctgcttgtacagacattttgtcccctgctgttttgtcctgggggtgggagggtggaggctaatggctgaaccggatgcactggttgggctagtatgtgttccaactctgggtgcttctctcttcactacctttgtctctagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagtaagggatcagcaaggatgtgggatcaggactggcctcccatttagccatgctgatctgtgtcccaaccctcaacctagttccacttccagatctgcctgtcctcagctcacctttctaccttctgggcctttcagccttgggcctgtcaatcttgcccactccatcaggcttcctgttctctcggtctggcccactttctttttatttttcttcttttttttttttttgagaaggagtctctctctctgtcacccaggctggagtgctgtggcgccatcttcactcactgtaacctctgcctcctgagttcaagcaattctcctgcctcagccttccaagtagctgggattataggcgcctgccaccaggcccagctgatttttctatttttagtagagacggggtttcgccaggctgttctcgaactcctgaactcaagtgatccacctgcctcggcttcccaaagtgctgggattacagtgtgagccaccacacccagctggtctggtccactttcttggccggatcattcatgacctttctcttgccaggttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtaaggcctggacctccatggtgctccagtgaccttcaaatccagcatccaaatgactggctcccaaacttagagcgatttctctacccaactatggattcctagagcaccattcccctggacctccagggtgccatggatcccacagttgtcgcttgaaacctttctaggggctgggcgaggtggctcactcatgcaaacccagcactttgggaagccgaggcgggtgatcacctgaggtcaggagtttaagaccaccctggccaacgtgttgaaaccctgtgtctactaaaatacaaaaaaaaaaaattatctgggcatgatggtgggtgtctgtaatcccagctactcaggaggctgagaagggagaatcagttgaacccgggagatggtggttgcggtgagccgagatcgcgccactgcactccagcctgggaggctgagcgagactccatctcgaaacaaaacaaaacaaaactatctaggctgggggtggtggttcatgtatgtatgtgtatatacatatatatgtgtttatatgtatatatatatacacacacacacatacatacacacacatacacacacaaattagctgggtgtggcacccgtgtagtcccagctactcaggaggctaatgtgggaggatcagttgaccctaggaagtcaaggctgcagtgagtcgtgattgcgccactgtactccagcccgagtgacagagtgacatcctgtctcaaaaacaaaaaaaaatctccccaaacctctctagttgcattcttcccgtcacccaactccaggattcctacaacaggaactagaagttccagaagcctgtgtgcaaggtccaggatcagttgctcttcctttgcaggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacggtaagccaccccagtctcccttcctgcaaagcagacctcagacctcttactagtttcaccaaagactgacagaagcccttcctgtccagctttccccagctagcctgcccttttgagcaactctggggaaccatgattccctatcttccctttccttcacaggtctgcacacctcattgccccttttgcaactactgaggcacttgcagctgcctcagacttctcagctccccttgagatgcctggatcttcacacccccaactccttagctactaaggaatgtgcccctcacagggctgacctacccacagctgcctctcccacatgtgacccttacctacactctctggggacccccagtgttgagcctttgtctctttgcctttgtccttaccctagaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcaggtgagtggagggcgggcacccccattccataccaggcctatcatctcctacatcggatggcttacatcactctacaccacgagggagcaggaaggtgttcagggtggaacctcggaagaggcacacccatccccttttgcaccatggaggcaggaagtgactaggtagcaacagaaaaccccaatgcctgaggctggactgcgatgcagaaaagcagggtcagtgcccagcagcatggctccaggcctagagagccagggcagagcctctgcaggagttatggggtgggtccgtgggtgggtgacttcttagatgagggtttcatgggaggtaccccgagggactctgaccatctgttcccacattcagcaagttcattcctgagggctcccagagagtggggctggttgccagtcagaagaacgacctggacgcagtggcactgatgcatcccgatggctctgctgttgtggtcgtgctaaaccggtgagggcaatggtgaggtctgggaagtgggctgaagacagcgttgggggccttggcaggatcacactctcagcttctcctccctgctccctagctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagcccagtcagtgtgagtggctttattctgggtggcagcaccccgtgtccggctgtaccaacaacgaggaggcacgggggcctctggaatgcatgagagtagaaaaaccagtcttgggagcgtgaggacaaatcattcctcttcatcctcctcagccatgcccagggtccgggtgcctggggcccgagcaggcgttgcccgctggatggagacaatgccgctgagcaaggcgtagcccaccatggctgccagtcctgccagcacagataggatctggttccggcgccggtatggctcctcctcagtctctgggcctgctggtgtctggcgttgcggtggtacctcagctgagggtcaaggaaggaaggtgtgttaggagaactagttcttggatccctgcccactctccccagggctgcccctcccatctgccccttacctccatcccaggggaagtagagactgagaatgtgggtacaataggcacagaggttgtgcagcccacgcaggtggacctgcagcttcccactgggcagctttgcctgcagcagcagggccaagtagctgaagacgaaggcgtccaaggaggcagggctggagcagagagagaagggtgggatggaggagaaccactggggtagaaggggtaaagatggagctggaggaagagtcagccttgggaggtgggctctgggcagcaggcggccaccagggaaggacaggacacacagttctagacctggtatggggagagatccccaggtggcgccagctggccctgaatagggctctatcccagggctgcataaagggcacactcagtgccccacagctcttcaggcccttcctgtgcctggctgccctcccaccctacccttttgtacctctgagaaggctctggccccacgcacagccccactgtcaccagggccagtatctgtctcagggacctcctatccagagcctgagccagccccagccccagccccagctccagctgctccatctgaacctgtatcttcttccaagccacccattaccctcttggagtcagactcacgcatctccaaagaagaacttttgagagcccaggcgctgagagagcagggtcagacactcccgagcctctcggtacagctgtaggggcgacacaggtaggcttgcagctgcgggaacagtgccacctccgcacctaagcactcccattcctggccagcatccttggggctcatctcatacaatagcccccggtctcagagctacctccttctccagctcttcctcgtcctcaggcctgtgctccccagtcagcagctgtagccgttccatgtactgccgctgcatgcggccaggcaggaagaagttgaggggaaagggcatagcctctgcataccacttccgggtcacttctacgtagttcttggtgtctatccaaaaagtatgtacctggattgggtgggcaggaagaaacaggcaggtctgagccagtgcacctgtctgattcaaggtgggcttctgacctccatgctctcctgagtctctgtgtgggtctgtgtgttcccgtcccctccccggctggccatggatgctgggaggtctgggcacactcaccagcaccgggatcaacttctcctccaggagagacatgaaggccagggtgtctgccccttgctgagctgacagatcataatcagcattgtacttctgtggaggaaatatccatggcgtggacgctggggagctgcaagggcacttcaccagggaggaaggagtcctgtctggtacccccctcactggcctctgagtgcagtggaggtacagcaaggaacttttcctgccaaggcccccttgcctgggcccagccagtagcctgttgctgttggcaaaaagcctgggccttggagcccgctggccgtcaaggtcctgggcccattgagaagaaggaagaaaggttgggccgcaaactaggagcagctcccagaatttccatggaaagctggaacaatgcctgctgacagcaactttctaacagtaactttcccgacccagacaccacaaagctagcacaacggagctcagatgcaggctaggactcggtccatgcctcaggaaccagggaaagccatcctcacactccctggatccagggaacccacgcccagggccccccagcttgttccctcagtgcccagctcttggctatttctttcacttcattccatcgcccagacaccattaccacatacacattccatccatacccccaggtctcagcctgccctaccttcccaggctccagtccctgttcctcagcatcccccaccacatcctgagtaagctttgtccccagataacctcttcagcatgatccttaaatctccctaagcctcagtttctcccctgtggaatgggggtaagaatctctttctctgaatgcccctgtgttaggaaataatttagaatacttcggaaacaaaaagctctgttcacacctaagcaatcagggcagtggcctggccttgccaggaacttaggcttttatctggatcctctttccaggcctctcaattaattccccaggtccttaacctttgggaaattagaaattaggaagagtgtcccacttctgacactgtgttccctcttggaacctgaccgtcaatgctagaagaacccttggaaaacatgctggcccagccctctagttttacaaataagggagtgcacagccctgagaggttacatggcctgcccaagatcacgcagtcaatggcagagtaaagagcatagcctaggcctccccactcctctagtaatgctctttcatcttctccaacctggctctaagccttgtccatcctgagccccatatctagcccaacctagtccctgaaaacaagaagtggcccttagaaatctctctccagtcccactatcagaggccaactgctgtcttccagtctccttcagcctgtgctcctctccctccctgactgacaggcagaaggtaccgtgcctctggatatccccacagtgccctgagctgcatctcttgccgactgctttaatacatcacagtgacattgtgtgtgtctctgccaccagactattgctccttgatgctctgggtcacctgcatctagcatggcatatatctagtgctcaataaatgtgtattgtacggaattgactgaacttctctcactggcagccccctctatccaaatcacccacctctttttgaaggtgggtgatgatcttgtgtggtactgaggtgacctctccatgactggtccaaagggcaggcagggttcctgatactgagagaaaagtataccaaccaggaccttagctgcctatccatacgtagttgcaacacattcctgcctatctcctgcttctcctcttgtacacacccttcctcacccccaagggatactgggtacctgaagggctctgccaggggttgctgatcttgtgctatatggtgcacaagaaacttttaagaaaaaagaaactttcaagacacactttccaaccacgaattctatctctgctccttttcatagcaataggttttctttttcttccctccacacttaaacatccattctcttatcacccacccccatcagactccttcccctgtgtttcttcagccactgctctgaccaaaatttgagtgaccaaaagtggtgtcagacccagtgaccatttctctgcctgcatttcacttgaccttgaagcagcaattaatctccataatcagcatcttcttgaatttttccctttgaggacattgctcttctactttgtgctctggttatcctttacaaacttttacacctctcctgaactggttaacagtagagggccccaagggtctcacgtaagccttctttgtttttgttttttttcttttcttttattttttatatatttttttgagacggagtcttgctctgttgcccgggctggagtgcagttgcacgatctcggctcactgcaagctctgcctcccaggttcatgctattctcctgcctcagcctcccgagtagctgggactacaggtgcctgccaccatgcctggctaattttctgtatttttaacagagacagggatacaccatgttagccaggatggtctcgatctcctgacctcgtgatctgcccacctcagcctcccaaaatgctgggattacaggcgtgagccactgctcccagccgtcatttttattttattttatttatttttttgagatggagtcttgctcttttgccaggctggagtgcatggtgcgatctcggctcattgcaacccccgcctcccaggttcaagcgattctcctgcctcagcctcctgagtggctgggactacaggtgcctgtcaccatgcctggctaattttctgtattttagcttgagacacggttttcaccttgttagccaggatggtctcgatctgtgagcctcgtgatctgcctgcgtcggcctcccaaagtgctgggattagcaggcagtgagccactgcacccggccatcaattttttttttttttaaatggagtcttgctctgtcacccaggttggagtgtaacagtgcaatcttggctcattgcaacctccgcctcttgggttcaagcgattctccagcctcagcctcctgagtagctgggactacaggtgcatgccaccacacccggctagtttttgtatttttagtagagacggggtttcaccatgttgtccaggatggtctcaaactcctgacctcaaatgatgtgcctgccttgaccttccaaagtgctgggattagaggcgtgaaccacattgcccaccgtaagccctcttttctacttccactctttctgtaggtggccctactactaacgtccatggctttaagtaccatctttctatgtgttaatgcataagtccagccttgacctctcttgagcgccatccaactcagcatatctggttggatgtctaatgagtatttcaaattcaacatggccacaactgaactaactgaactcttttttttttttaggcagagttttgctcttgttgcccaggctggagtgcaatggcgagatcttggctcactgcaacctctgcctccagggttcaagcgattctcctgcctcagcctcccgagtagatgggattataggccccgctacccggctaatgtttttgtatttttagtagagacagggtgttgccatattgaccaggctggtctgcaaatcctgacctcaggtgatccccctgcctcggcctcccaaagtgctggaattacaggcgtgagtcactgccccggccacacaactgagctcttcattcccaccccagcaccggttatttttccactgttcccatctcaatggcacctccattacccatttgcacattccaaaagcccaggaaccatggtgacttcttttcccatatccaacacaaccaatcctatcttgaattcatccacgtcccaccacctccccagctacctagttccagccaccctctctccacaacctctgaatcaatctttcacttttcccagcaatccattctccactcagcaaaatgatgataaagcacgtcacatcaaggctctgcctcaatttaatggcttcccattgtatttagaatcatctccaagctctcagagactatggtcagctacaatctggcccaccttctgttccagccaaatttcctcacagcacaaggacgtttgcacctgctattttccaagcacgaaaccctcgggcagatatatctggtgctgtcacctaatttcaggttttaactccactctcaccatttcagtgaggacctaatccccatcgcagtcattctatcacatagctttattttattttattttatttttttttgagatacagtctggctctctcacccaggctggagtgcagtggtgtgatctgggctcactgcaaacttccatctcctgcgttcaagcgattctcctgcctcagcttcccgagtagctgggattacaggtgtctgccatcacgcctgactaagttttgtattttcagtagagacggggttttgccatgttagccaggctggtctcgaactccttgacctcaagtgatccacctgcctcagcctcccacaatgttggatttacaggcgtgagccactgctcccggccacatagccatgttttaagcatgtatcactatctaaaattattttttgtttatacgtttgtgtcgtcctgtagaatgtaaggtcacaagatcagggacttgctcattgcactgggtctaacacacagtgcttcaacaaacactcgttaagatactaacgtggcagagtggggccttgtaaacagtttcaggaccctgtgcttgtaagagcaacgtggtgccctcccaaggaagacagggaggatgcaggagcactgcccagagatggcgtcaggctgcaagacatcttgaataattcaccatcgtaacaacccagcctcaggaagagatggggcaaggccagaacgaaacattaggtaagaggcggtggacaatgggattcccacagggcagcttttgggcactggacgttccctaacctgaggctctctgaagaggaaggttaggaatcctctcagcttcggtgggctggactcactgtgggaattcaatcgcccccccccacccactaagggtgtgctggcgggaaagcgctgagacgcatgcgtagttctcgcgtctggcacccgctccctttccaatacgcttgcgccccgtctgtgctacggatggtcagggagagttgtccgtcttcaaatggaccaatgagacttgtggaggggctctgagtcccgcctctggatgagtgaccgtctcttttccaagtgaggcccgcccgcctccccagggctgcttttctcgcggcacgggtggtggggctgcttcttgacttccgcgcctagtgcgcagggccccattctccagtcccgcccacgcgcctttggaggctgcggtgggatttccttttgccttcggttggggctgctgtttctcttcgccgacggtaggcattataaatatttcgccctttgaattttagctctcccctcccggcgttcgcacttagcctttttcatcattatcattatttttaatggtaaggatggtcaaattttactaatgaagcgattataaaatcttcaagtctttgtatccacttgctttttgagggctggagtgggaagaaaggtatataattcattcattcttcggacatgtgacaaacgttcacggagcgcggcaacgagcgccggtgtcgcgatgcgcactggggctgcacatgggagccaggatggactggactggtccctgccctgcccgctgacgattggcaggccactgcctttgatgagctgggcgctataactgccattaagccatttgtacaattaatcacaacagtgataagagcgacaaaggagctattcgggtggctgaggcggaggatcttttgagcctaggagttcgagaccagcctggggaatagagcgagatcctgtctcaaaaaaagggggagaaaaagtgacaaagaagttgtgagggctatgagtttgcgttagaggaggtgtgtgaggtggggaggcggcagggggcgcggttgttcttagagaaagtgacatccgggctaattttgaaagaataagtgttaactaggctaagcggtgggaaagagtagtgtgtgctgaaggaagggaagaggaacctggcaagttcatggcaggcctgtaattggatgctggtctccctccacacttgactcctacagtctgatttctacacagcacccaaagtgatctcttaaaaatacacatgtgatcttgtcacttcccagcaccaaactctacagtgaatttccatcttagaacaaaattcagactccttaccatggccaccaagaccctacacaatctggcctcccaatttccttttccaaggtcaccttttaccactatccatctcactcacactgctccagcactctctatgcttttatttttctttcttcctctttctttctttctttctttttctttttctttctttctttccttccttctctctctctctctttcttttcttttctctttcttttttttttttttgagatgcagtcttgctctgtcacccaggtgtgtgatcttggctcactgtcaacccagagcagtgggggtgatcttggctcactgcagcctccgccttccaggttcaagcaattctgcctcagcctcctgagtagcagggattagcgccatcagtcccagctaatttttgtatttttagtagagatttcatcatgttgtccaggctggtctcgaactcctgacttcaagtgatctgcccatctcagcctcctaaagtgctgagattacaggcaggagccaccacacctggctcagtatttgttttatttttgttgtgtttatttatttagagacaaggtcttgctctgttgctcaggctggagtgctgtggcaggaacacagctcactgcagcctccactacctcaattcaggcgatcctcccacctcagctgagactacaggtgtgcatcaccatgcctggttaattttttcgttttaccatgttggccaggtttgtctcgaactcttgggctctgctgtcctcctaccttagcctcccaaagtgctaggattgtaggcgtgagccactgtgcccagctggtgttcagtatttgaatccatatttcctgtagccgcaaccaaagttccactgttaggtctcactttgactttaataattgtgttcaggctgggcacactggctcaagcctgtaatctcagcagtttgggaggccgaggtgggtggatcacatgaggtcaggtgtttgagaccagcctgtccaacatggcgaaaccctgtctctactaaaaatacaaaacattctccgatcgtagtggcgggcgcctgtaatcccagctacttgggaggctgaaggaggagaattgcttgaacctgggaggcagaggttgcagtgagcggagatcacgtcattgcactccagcctgggctacagagcgagactctgtctcaataagtaaataaataaataaataaataaataaataaataaataattgtgttcggagtcagcatcattcttcctgaagttcaccactcctttgccaagaacacttcctgaaacactgacaagcaggaccttgaataatggggtatggttggtaacaactcactcattcaacaaacattgagcacctattttgtgcttgcctctaaatgaagagctggttgtattatttatttttttaggtgacagggctttccctatgttgcccaggttcgtctcaaactcctgggctcaaaagatcctcatcttctcaagtggttgaatatacacgctccagcgaccatgcctggctgaatgaagagctttgagattttgaagaaacaggaaccatgaaatttgctttgcaactgtttgcaacctttaaggaagactgaaaaggcattcctgaagcatgtgagaagcagtctgtgtgacctgatgactcagaactgcttggaatttagattaggacagatatgagcttaggcttcactctgccacatatttaacttctctaagtcttagttttcttttctttttttttttttttgagatggagtctcgctctgtcacccaggctggagtgcagtggcacgatcttggctcactgcaagctccgcctcccgggttcacgccatcctcctgcctcagccacccgagtagcggggactacagtcgcacaccgccacgcctggctaaatttttgtatttttagtagagacgggtttcaccgtgttagccaggatggtctcgatctcctaaccttgtgatccgcccgcttcggcctcccaaagtgttgggattacaggtgtgagccatcgcgtctggcctctaagtcttaattttcttatctgtaatgtagagttgtcaggctagtgcatgtaataagcactcatgaaagactgactattatcttgcgaaaaattggaagagatcatatgaggtacaaggaccttccatctgcactgggctgtacattgaatgttgtggttactgttgaagcattggtaaatcacatgaatccaatggtaaaaccacaccctaaggtcagactcagtggctcacgcctgtaattccagcactttgggaaaccaaggcaagaggattgcttgagctcaggagttcgataccagtctaggcaacctgtttctataaaaagttaaaaaattagcttggtgtggtggtgtgcacttctggtcccagctactcaggaggctaaggtgggaggatcccttgagcccaggtggtcgaggctgcagtgagccaggatcacaccattgcattccagcctggatgacagtgtgaggccctgtcttaaaaaagacaaaaacaaaccaaaaaaacccacaccctagtgggtaaggggcagcagaggtcccacccaagagtgaaatcatttttggtgtcagtaatcagggagagtaatataccctcaggccagaaactagaggtgagtgatggctcacgcctataatcccagcaccttgggaggctgaggcaggtggattgcttgacctcaggaattcgagaccagcctgggcaacataggaagaccccatctctaaaaataaatttcaaaaattagttgggcatggtggcatgtacctgtagtcccagctactatggaggctgaggtgggaggatccaattgagcctgagaggtcaaagctgcagtgaattgtgattgtgccaagaccctgtctctaaaataaaataaaattacaattaaaaactagagctgaaaggacaggttctaggttaactggtagtagttgttcattcatctgtataaaaagtacttttttgagcacctactctgtgctgtgcatggaatgaacaaatgtctttatttgacttgaacacagaccatagaagtaactaacagtggaactgggagtggcactctccatgggacctaaagacctaggagcctgtgatatgatacctgtcagtgtgaaagggctggagtttgctatctgagccagacctacactccaagacaggtcttgctgggatgggatcaaaattgctgctgggtcagccgaactatttttgtacttctgcttttcacttatatttatgtcttccctaggtatatagctgtctttttttgtttgcttgtttttctgagtcagagtcttgctctgtcactcaggctggagtgcagtggcgcgtctcggctcactgcaacatccacctcgggttcaaacgattctcctgcctcagattcccgagtagctaggattacaggtgcccaccaccatgcccggctaattttttgtatttttaatagagatggggtttagtagagacagggttcactgtgttatccaggatggtctccatctcctgacctcgtgatctgcccgcctcagcttcccaaagtgctgggattacaggcatgagccactgcgcccggccactggagaccccttttctaccaaaaaaaataaaaataaataaataaatatatatatatatttgtaaagacggagttctctatgttgcccaggctggtcttgaactcctggcctcaagggatcctcttgtctcagcttcttaaaatgatggggttacaggcatgagccaccgtaccaggccttagcaaactctttttcaagtgctatacaaaggggacagaggaacgtgattgtggccacctagtatgcccctattggccactgcagtgaggcctaggtgtttgaagagaggcacctagggtagtggctctcaggcacaccctaggggcattttggaaatttgtggggcatctttaatagagacaatgattaggaggcactttgagaatttagtggatggagcccaggaaaggtagacattctgcaacatgtagtacattcatacacaaggaagaattcactcatttcacctgacttacaaatataaaatcaaatataaatcagtaggactttattataaaatattgcagaaaagtttcacaagaattttttgtagaaaaagcccaatcagggccgggcatggtggctcatgcctgtaatctcaggactttgggaggccgaggcgggtgaatcacctgaggtcaggagtttgggacaagcctggccaacatggtaaaaccccgtcttgactaaaaatacaaaaatcagccaggcatggtggtgtgcgtctgtaatcccaggtactcgggaggctgaggcaggagaatcgcttgaacccaggaggcggggtttgcagtgagccaagatcaagccactgtgctccagcctgggtgaaagagcaagactccgtcgaaagaaagaaaggaagggaggaagggaggaagggagggagggagggagggagggaaagaaagaaaaaagagaagagaggggaggggagggaagaggagaaaagaaggaaggaaggcaggaaggaaggaatccagtcgagatcgttaaatttgttgtaattttgtctttggcacaataaacattagtccttattaaaactaactttacttagaaaataaaatagaaaatcctcaaggacaaatcacattgtacaggaaatatgaaatgtgaaataatatccaccttggaaattttttttttttgaaacagagtgttactctgtctcccaggctggagtgcagtggcacaatcttgggtctctgcaacctctgactcctgggttcaagtgattctcgtacctcagcttcccaagtagctgggattacaggcgtgcaccaccacacccagctaatttttgtattttcatttcagatgggattttgccatgttggccatgaacacctgtcctcaagctatccactgcctcagcctcccaaagtgctgggattataggcatgagctaccgtgcccagccaccctggaaaaattttacaaaatgaagtataaaaagaagaaaagaggggcgggcatggtgccttgtgcgtgtaatcctagaactctgcaaagccgaggcaggagatcctttgggtttaggagtttgagaccaacctgcacaacatagcaagaccctatttctacaaaaaatacagacactaggctgggcgtggtggctcacgtgtaatcccagcactttgggaagctgaggccagcagatcacgaggtcaggagatggagaccatcctggctaacatagtgaaaccctgtctctactaaaaatacaaaaaattagccgggtgtggtggcaggcgcctgtagtcccagctactcaggaggctgaggcaggagaatggcgtgaatccgggaggcggagcttgcagtgagccgagatcgtgccactgcactccagcctgggagacagagcaagactccgtctcaaaaaaaaaaaaattttgctcagtacctggccaaaaaagaagcagctcactccctgtacacagaggggtaagagaaaggagatggttgaacttctaaactcgctaaagcaggagaggcaaatgtggaatgtgctcaggaaatatctgtgagatgaatgaatttgagggaagtaaggtactagataattacctgccctacccagaacaaatcctgtgcaacgtttccttgaagagcaggaagtcaggccgggtgctgtgctcacgcctgtaatcccagccctttgggaggccaaagtgtgcgaatcacctgaggtcaggagattgagaccagtctggctaacatggtgaaaccccatctctactaaaatacaaaaattagccgggcgtggtggtgcgtgcctgtagtcccaactacttgggaggctgaggcaggagaattgcttgaacctgggaggcagaggttgcggtgagctgagatcggccactgcactccagactgggtgacagagtgagacaacatctcaaaaaaacaaaaaaaaaagagaaaagcaggaagtctggaaggggtggctactgacatagtgaagcaactagttcaattctacaacttgacaactacccctgtgccaggctgtctacaaggatatttagaatgtgtaagacattccttcaaggaactccaggaacagaggcctgacatgttgcaatgtttagtgtcaagcagtgtactagagacacattatcacactcaaacctcacaacagttctatgaggtaggagttatcactccccttttatagatgaaatagaggcttagagtgattgatttactgaaggtcaaacagccagtaaatggtgtagcaaggattccaaccttgccgtctcactaaaactgtacaaaaaaagatacaaacaacagacaaatagttcccaggcgcctcccaagttgccaggcactgcatttacctcactgacccctttgaggttgtggcattgcctccattttctaggtgaggaaataggctgagagctggggttagtctggtcatgactgtgtgtgccactcccaccaaatctcatttgatgtggttcatgaggcaaatggcatggacaacttccttcacatgtccactaagcatatggccttttacaacactttctggtttctgaactactttaaaacctcactgtcctgtgaggaaggaagaacagttattacaatctgcatctggaagccaattgccctttagagatatggctgcaattgcctcactgcctgtgtcatgtgactctccgaggcccttaatgagtaaatgaggggtgctgcaggggagccaagctgaccactcccctccctccagtcctgccaccccactgccagtgtcccaccctccttgcgccctacacttcactggctaataacccccctcactttttcctgtgttgaaggcatcctggataattccccacccacgaatggtccctcctcatctcagagagctctccatgcacacctgttactgtttctgtttttacctgtaaatatctgtgtctgacttccatgcttcatgcacctctatagggcaaagactgtgtcttaaacatcacggtagcctcagcatgttgtgcaatgaaggtttttttgtttttgttctttgttttttttttggtattagctttatttgtatcattttgaaatttttatcaaaaaagcagcgtgcctgctgtggttcccatcctctgggatttaggaatctttacccgattctccatccaagtctgtctttcgtattctaggctcttcctaaagttgtcattcacatataccctccagaattttatagggtgtataatctgtaacaactcggaggaagccaattgccctttagaaatatggctgcaattgcctcacttcctgtgtcatgtgactctcctagtcatcacatgacccatccacattgggaagccagaattacttgcaggagtaacctagtgcctatagctatggcaggtatcctgcatccttgttttttgtttagtggatcctctatccttcagagactctggaacccctgtgctcttctcctcatctagtgaccctgaggtgatggagttttcaagtccttccagagaggtaagagagagagctcccaatcagcattgtcacagtgcttctggaatcctggcactggaatttaatgaatgacagactctctttgaatccagggccatcatggctctttgagcaaggcacagatggagggaggggtcgaagttgaaatgggtgggaagagtggtggggagcatcctgatttggggtgggcagagagttgtcatcagaagggttgcagggagagctgcacccaggtgtctgtgggccttgtcctaatgaatgtgggagaccaggccatgggcacccaaaggcagctaagccctgcccgggagagtagttgaggggtggagagggacttgcttttcagtcattcctcattctgtcctcaggaatgtcccaagccttcgggtagggtaagcatcatggctggcagcctcacaggattgcttctacttcaggcatgtcgtgggcagtcagatgagtgagtcaaggcagtggggaggtagcacagagcctcccttctgcctcatagtcctttggtagccttccagtaagctggtggtagacttttagtaggtgctcaataaatccttttgagtgactgagaccaactttggggtgaggatttttgaaaccgtcttcagtctctccaaacagctgtgtccgttctccacatccttgtcagacctcacctctgcttgtgctccctccctcccaggtggtgcccctgcatccctaaaagcttcagtacagctcggtggtctgtgtctgcaatgccacatactgtgactcttgaccccccgacctttcctgccctaggtgccttcagccgctacaagagcagaagcagtgggcattggatggagctgagtacaggaccatacaggctaattgcaccggcacaggtaaccattacacccttcaccccccgggccaggctgggtcctcctagaggtaaacggtgtcagtgatcaccatggagtttctccctgggcactgataaccctgtggatgtcctcaggcctgctactgatcctgcagccagaagttccagaaagtgaagggatttggaggggccgtgacagatgcaggtgccctcaacatccttgccctgtcaccccctgcccagaatttgctacttaaatggtacttctctgaagaagatgaggaggaaggggacaggatgacatagagccactgacacttttctttgccaattcttgtggaccctgacttctgcccatccctgacatttggttcctgtcttaatgccagtgaaataagatttcgccgcctatcatctgctaactgctacggactcaggctcagaaaggcctgcgcttcacccaggtgccagcctccacaggttccaacccaggagcccaagttccttttggccctgactcagacactattaggactggcaagtgataagcagagtcccatactctcctattgactcggactaccatatcttgatcatccttttctgtaggaatcggatataacatcatctgggtacccatggccagctgtgacttctccatccgcacctacacctatgcagacacccctgatgatttccagttgcacaacttcagcctcccagaggaagataccaagctcaaggtaggcattctagctttttcaggccctgagggccctgatgtctgggggttgagaaactgtagggtaggtctgcttgtacagacattttgtcccctgctgttttgtcctgggggtgggagggtgggggctaatggctgaaccggatgcactggttgggctagtatgtgttccaactctgggtgcttctctcttcactacctttgtctctagatacccctgattcaccgagccctgcagttggcccagcgtcccgtttcactccttgccagcccctggacatcacccacttggctcaagaccaagggagcggggaatgggaaggggccactcaagggacagcccagagacatctaccaccagacctgggccagatacattgtgaagtaagggatcagcaaggatgtgggatcaggactggcctcccctttggccatgctgatctgtgtcccaaccctcaacctggttccacttccagatctgcctgtcctcagctcacctttctaccttctgggcctttcaaacttggatctgtcagtcttgcccactccatcaggcttcctgttctctcggtctggcccactttcttggctggatcactcatgacctttctcttgccaggttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctacccttgccaacggtactcaccacaatgtccgcctactcatgctggatgaccaacgcttgctgctgccccactgggcaaaggtggtaaggcctggacctccatggtgctccagtgaccttcaaatccagcatccaaatgattggctcccaaacttagagggatttttctacccaactatggatcctagagcaccattccccgggacctccagggtgccatggatcccacagttgggacttgaaacctctctaggctgggggtggtagctcatggctataattccagcactttgggaacccaaggtgggtggatcacttgaacctaaggagttcaagatgagcctgggaaacatggtgaaaccctaactctacaaaaaaaaaaatagaaaagttagccgggtgtggtggtggcacgcctatagtcccaagtattctggaggctaaggcgggaggtttagttgagcctagaatttcaggctgcagtgagctatgattgtgccactgtactccagcctgtgtgacagagggagaccctgtctcaaaaacaaaaacaaaaaatccctcccaaaacctctgtagttgcattcttcccaccacctaattcaggattcctacaagaggaactagaagttccagaagcctgtgggcagggtccagggtgacttgttcttcctttgcaggtactgacagacccagaagcagctaagtatgttcatggtattgctgtacattggtacctggactttctggctccagccaaagccaccctaagggagacacaccacctgttccccaacaccatgctctttgcctcagaggcctgtgtgggttccaagttctgggagcagagtgtgcggctaggctcctgggatcgagggatgcagtacagccacagcatcatcacagtaagccaccccagtctcccttcctgcaaaggaggacctcagacccattagtagtctcaccaaagactgatagaagcccttcctgtccagctttccccaggtagcctgcccttttgggcaactctggggaaccatgattccctgtcttgcctttccttcacaggtctgcacacctcattgccccttttgcaactactgaggcacttgcagctgcctcagacttctcagctccccttgagatgcctggatcttcacacccccaactccttagctactaaggaatgtgccctcacagggctgacctacccacagctgcctctcccacacgtgacccttacctacactctctggggacccccagtgttgcgcctttgtctctttgcctttgtccttaccctagaacctcctgtaccatgtggtcggctggaccgactggaacccatcattgtagacatcaccaagcacacgttttacaaacagcccatgttctaccaccttggccacttcaggtgagtggagggcggggcacccccattccataccaggcctatcatctcctacatcggatggcttacatcactctacaccacgagggagcaggaaggtgttcagggtggaacctcggaagaggcacacccatccccttttgcaccatggaggcaggaagtgactaggtagcaacagaaaaccccaatgcctgaggctggactgcgatgcagaaaagcagggtcagtgcccagcagcatggctccaggcctagagagccagggcagagcctttgcaggagttatggggtgggtccgtgggtgggcgacttcttagatgagggtttcatgggaggtaccccgagggactctgaccatctgttcccacattcagcaagttcattcctgagggctcccagagagtggggctggttgccagtcagaagaacgacccggacgcagtggcactgatgcatcccgatggctctcctgttgtggtcgtcctaaaccggtgagggcaatggtgaggtctgggaagtgggctgaagacagcgttgggggccttggcaggatcacactctcagcttctcctccctgctccctagctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacaacagggccagcgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagcccagtcagtgtgagtggctttattctgggtggcagcaccccgtgtccggctgtaccaacaacgaggaggcacgggggcctctggaatgcatgagagtagaaaaaccagtcttgggagcgtgaggacaaatcattcctcttcatcctcctcagccatgcccagggtccgggtgcctggggcccgagcaggcgttgcccgctggatggagacaatgccgctgagcaaggcgtagcccaccatggctgccagtcctgccagcacagataggatctggttccggcgccggtatggctcctcctcagtctctgggcctgctggtgtctggcgttgcggtggtacctcagctgagggtcaaggaaggaaggtgtgttaggagaactagttcttggatccctgcccactctccccagggctgcccctcccatctgccccttacctccatcccaggggaagtagagactgagaatgtgggtacaataggcacagaggttgtgcagcccacgcaggtggacctgcagcttcccactgggcagctttgcctgcagcagcagggccaagtagctgaagacgaaggcgtccaaggaggcagggctggagcagagagagaagggtgggatggaggagaaccactggggtagaaggggtaaagatggagctggaggaagagtcagccttgggaggtgggctctgggcagcaggcggccaccagggaaggacaggacacacagttctagacctggtatggggagagatccccaggtggcgccagctggccctgaatagggctctatcccagggctgcataaagggcacattcagtgccccacagctcttcaggcccctcctgtgcctggctgccctcccaccctacccttttgtacctctgagaaggctctggcccccacacagtcacactgtcactagggccagtttctatcccagggacctcctatccagagcctgagccagccccagccccagccccagctccagctgctccatctgaacctgtatcttcttccaagccacccattaccctcttggagtcagactcacgcatctccaaagaagaacttttgagagcccaggcgctgagagagcagggtcagacactcccgagcctctcggtacagctgtaggggcgacacaggtaggcttgcagctgtgggaacagtgccacctccccacctaagcactcccattcctggccagcatccttggggctcatctcatacaatagcccccggtctcagagctacctccttctccagctcttcctcgtcctcaggcctgtgctccccagtcagcagctgtagccgttccatgtactgccgctgcatgcggccaggcaggaagaagttgaggggaaagggcatagcctctgcataccacttccgggtcacttccacgtagttcttggtgtctatccaaaaagtatgtacctggattgggtgggcaggaagaaacaggtaggtctgagccagtgcacctgtctgattcaaggtgggcttctgacccccatgctttcctgagcctgtgtgtgggtctgtgtgttcccgaaccctccccggctggccatggatgctgggaggtctgggcacactcaccagcaccgggagcaacttctcctccaggagagacatgaaggccagggtgtctgccccttgccgagctgacagatcataatcagcattgtacttctgtggaggaaatatccatggcgtggacactagggagctgcaagggcacttcaccagggaggaaggagtcctgtctggtacccccctcactggcctctgagtgcagtggaggtacagcaaggaacttttcctgccaaggcccccttgcctgggcccagccagtagcctgttgctgttggcgaaaagcctgggccttggagcctcctggccgtgaaggtccagcgcccaatgcagggaaggaaggaaggctccgccgcaaactaggagcagctcccagaatttccatggaaagctggaacaacgcccgctgacggcaactttctaacagtaacttccccgacccagacaccacaaagctagcacaacggagctcagatgcaggctaggactcggtccatgcctcaggaaccagagaaagccatcctcacactccctggatccagggaacccacgcccagggccccccagcttgttccctcagtgcccagctcttggctatttctttcacttcattccatcgctcagacaccattaccacatacacattccacccatacccccaggtctcagcctgccctaccttcccaggctccagtccctgttcctcagcatccccctccacatcctgagtaagctttgtccccagataacctcttcagcatgatccttaaatctccctaagcctcagtttctcccctgtggaatgggggtaagaatctctttctctgaatgcccctgtgttaggaaataatttagaatactttggaaactggaaaagctctgttcacacctaagcaatcagggcagtggcctcggctctgccaggaactttggcttttatctggatcctctctttccaggcctctcaattaattccccaggtcctcaacctttgggaagttagaaatgaggaagagtgtcctacttctgacactgttccctcttggaacctgaccgtcaatgctagaagaacccttggaaaacatgctggcccagccctctagttttacaaataagggagtgcacagccctgagaggttacatggcctgcccgagatcacatagtcaatggcagagtaaagagcatagcctaggcctccccactcctctagtaatgctctttcatcttctccaacctggctctaagccttgtccatcctgagccccatatctagcccaacctagtccctgaaaacaggaagtggcccttagaaatctctctccagtcccaccatcagaggccaactgctgtcttccactctccttcagcctgtgctcctctccctccctgcctcacagtgccctaagtttcatctcttgccgactgctttaatacatcacagtgacattgtgtgtgtctctgccacaagactgttgctccttgatgctctgggtcacctgcatctagcatggcatatatctggtgctcaataaatgtgtattgtatagaattgactgaacttctctcactggcagccccctctatccaagtcacctacctcttttcgaaggtgggtgatgatcttgtgtggaactgagatgacctctccatgactggtccgaagggcaggcagagttcctgatattgagagggaagtataccaaccagacccttagctgcctagtcatacatagttgcaacacattcctgcctatttcttgcttctccccttgtacacacccttccttacccccaagggatactgggtacctgaagggctctgccaggggttgctgatcttgtgtaccttcagtggagcaccagtaaatctggcataggtctgcagggaaggaagcagaagtcagagaggcagagccatgtcccccacagtggtatcaagaagagaaataacatttattgaataccttatgtgccattccctatacttagtatcttagtctcgaaaaaaaagggggagtcactaccatttctattttacaaatggacaacacagggctcagagagattccggacatgtctgtgattacaggacagccaggaaaatcctttcctctatctcctctcttgccatctacctggtgaacatctatcctcagaagcttttcctaaccagtatccctcttcttggaagcactgataatgcccttctctttggcagtatctgtggtctgttcctactttaacttatatgtgaatattttattatatttatttgcttacctatctgcctttctgtaaactcgaagggaggaaccaaattttattcatctttgcctggcaatgtttaatatttggaagaaatctaatagatcctctttaaagcaaggagtcaatgaataaatgaagcaaaggaagtctgattgctaagctctttatacaaagcctggctgtgagtcgtttggtctaacaaacaatagcctcagtaaatgctgttaagtgaggaggaagaggagaagggggctgaggagaaggcaggaattccaagctagagtaggtccctgagacagtccagcctagtaaacctccgaatggagaaaaaaaatgcccaaagagaaaggatctccgcaaggtcacacagccagtgaatggatgagcaaggactagaacccatcacctgaactcccaaccaggcgtcttttcattgctgcattaagcctagaaaactacagcatagggcactgaggccatatcggccacgtacgctctttgactgcctcagtttcccccactgcgcccgtgctgagtagccctggcaaggtttggatgcttggctagttcggccccctccccagtcatcagggcacagcagagggcgccggcgccacccctcaccagcacggccaggctgtccaggtccactgacggcagcccccagccccctgaccagcagaacagctccatgggcgccgccatcttgcccaccctctgtcccggaaacacttccttgtgtgcttctgccctcccctgcctgggcccgccccccgccaccgcccctccgatcgttgcggtcagggggcctggggagatccccggggaggcgaggcttcttctggcccgactggcagctgaactgcgggggactgggccgcgggcctcgggggagggcggccgccggcccatccagaggtggcccacgtagcgggacagcgctgtcggcccggcgcgcctcggagtgtcacggcgcctcgtccaagtggagccccgaacccctgaaggcgcggcaggctctggagagcggggtcttgtgcgcctgggccaggtctgggggctcttgccaaactgcacgtggcctgtactgctccagggccccttggggctcgtccccgagcggggactgcgggggggtcccccgagcagcatgttttccacagcgcgttatgtttggagcgggccctgcgccgcctgtcgccatggaaacaaaacaggggcggtggcggcggccggagcggaggccgggctggggcttgggtgggggaggggaagagaggctcgcaggctgtcgcttaggtgacgggaactcaggcgcccctctgcttcatccgggtcacggcccgtccgctagtacccacagtgttccacagtctggtccttggctcctcgcctgtacccctggtcttctgcgcctgtccctggtgtccctttcctcttttttggtttcttcactctgacctcactgaccactgctttagattctcccttcagttcccgtcagacgcttccagactcccaagctttcctacgaatgagggaaaatggagaaacaggcacttgtcaggggacccccaccctaataaagagcacttgctccgccagaacagcaaaattcatgccatgtgggcatccctgggcactatagcaagctagttgcggccactcccttggcatcctttcctgccagctgtggaataatgcccactgtctagcactgcccctgccaggggttcttgccttccacaatcgtggcttccagaaaacagtggcattcggtagcgctgtgtgccgagacccccaacaatgatgactgcggagagggaggcacctggggggaggatcattagggagaggtagaaagcagggaggcctccaggattctttcccagtgcccctggttcccagagctgatgatgcctccagggtgattggcagctctttgtttcagccccccctcacccgcctgctggccccccctccaattctgtccctccgccccccggctcctgctctctccgcctagccttttcccctcccagctgcctgcctgccaggggtagtgagccggctgagaggcatggagacgcaggaacttcggggggccctggctcttctcctcctttgctttttcacatctgccagtcaggatctgcagggtaagcctgtctccatcctcttagaccgctctctgcttcttccccatttgccctcagcccaagtagcagagaacatgtgggcaaggggagaggggaagagtccagaaattgagccagaggaaaacttaagactgcctagagttggtgaaattatggcctggtggagaggagtgggcaccggagagtagtgggggagctggaacaggacagggtcaggcatgaggccagggcagaggactcaggaactggatgctcaggctgcctggctggggtggttcctgagcatctgtaggcaccctagggtctgggagagcagcctgaaggtgggcgtactgtaggtccctgggcttcgtccccctgggctcctggctgcccaaggacagggcgggggtggggaccaagaagcctgggctctcccggaggtctggtggtgggatgggccgatggatgtgggagaggtagctggagcagatgacacaggcatagattctttactggctggagggaaaaccacagattggccaggacacaggaggcagaggagctgggtcctacattcccgtgggagatagcccagatggtgggagggttagagtcctcagtgggcagctgttcttatccagagcgagtgtgctgcgcccctaggatgggaggaggggaactagggtgtggaacgagccaaacccaggagtggaaagagaagctgccttccatttctacgttgtggacaccaggtgccactcctgtgggggatcagcacagcatctcctttgcgccacctggtgggggcatctcaaatttgtggggtgctgtttctgtgtttccaagtgagtctcaaacctgcaggttcctggaaggtagtaataggcaataatggggaaaggaaggcacagtgcctctgtcctctgagagcaacaagggttgaggtttttttttgttttcgttttgtctttttgagatggagtttcactcttgttgcccaggctggagtgcaatggcgcgatctcggctcactgcaacctccacctctcaggttcaagcgagtctcctgcctcagcctcctgagtagctgggattacaggtgcccgccaccacgcccggctaggtggtttttttttcttttttgttgttgtttttgtatttttagtagacacggggtttcaccgtgttggccaggctggtctcgaactcctgaccttaggcgatccacctgcctcaacctcccaaggtgttgggattacaggtgcgagccaccatgcccagccaagggttgaattttgaggtgactaacgctgcctggtggtgggggtagggggtgatgtgggctcggggagggcataccttagcatttgatcccattctctaaccttgataacccctgcctgaccagtaattgacctgctgactgtgggcgagtctcggcagatggtagctgtggcagagaagatccggacagccttgctcactgctggggacatctacctcttatccaccttccgcctgccccccaagcagggtggtgtcctctttggcctctattctcgccaagacaacactcgatggctggaggcctctgttgtaggcaagatcaacaaaggtgactggtgggcatctcctttcttgcaatggtgacctccttaagcactctccctcatccctacttcccaagtgcttcctcattctccttggcctccattagggaccaaggtcccatcaggctgtgcccagacatcaggggcacctggtatggggtgcttataaggtgctttaggagcccatgtaccaaacaatagggtgaggcttgagtcctgatatttatagggatgggaggttgtacttggggatgcctggaaggagggtgaggcatgtctctgtgcctcagcggtgatacagaaaggtctttcagatccctggaacagtcagtggccacaaaaccagctgtcaccctaggagaggatacagtcatctgataccaatgaagactaagaaagtatttgtctctaaaccaattatttttttaaatggagtcttgctttgtcacctaggctggagtgcagtggtacaatctcagctcactgcaacctctgcctcctgggttcaagcaattctcctatctcagcctcctgagtagctgggactacaggtgcccaccaccatgcctggctaacttttgtatttttagtagaaacagggttttgccatgttgaccaggctgttctcgaacttctgacctcaagtgatctgcccaccttggccttctaaagtgctgggattacaggtgtgagccaccgtgcctggcctctaaagcaatttcaaaaacatttattgtgtacctattaaatacaaggtgttgagagccacatagtagaattagggataggggccctagagatgtggtatctagcagagttgatccaaggctcagctgggaggcagggggtgggggatagcgcccatactgatctcccctcctgggcagtactggtgcgataccagcgggaggatggcaaagtccacgccgtgaacctacagcaagcgggcctggctgatgggcgcacacacacagttctcctgcgactccgaggtccctccagacccagccctgccctacatctctacgtggactgcaaactgggtgaccaacatgcaggccttccagcactggcccccattcctccagcggaggtcgatgggctggagattaggactggacagaaggcgtatttgaggatgcaggtgagccgggaggagcctctgagttctgtggaaatagagtttgcaccagctggggaaggggttggtagaccttcccaccttactctgctgctatccccccagggctttgtggaatctatgaaaattattctgggtgggtccatggcccgggtaggagccctgagtgagtgtccattccaaggggacgagtccatccacagtgcaggtaacacagagacttgtttgctgacattggaccacggatcctgtggcctttggtgactcctgtcttcttgatctccctctcccttaaaacccattcctttggctcacctgttcctagggtttctaaccctgttattccaaatctttcacctgactccacaatctccaatacacccatccgagaaaaaaagtgatctaagagaagaattggttaattgcttggccttggctgaccaagagatactggtctcgagtattttttttttttttggtgatggagtcttgtcctgttgcccaggccggagtgcagtggtgcaatctgggtcactgcagtctccactgagttcaagtgattctcctgcctcagcctcccaagcagctgggattacaggcggccctccaccatgcctagctaattttgcatttttagtagagatgggatttcaccacgttggccaggctggtctcaaacacctaatctcaagtgatgcacccacctcggcctctcaaagtgctgggattacaggcgtaagccaccgcgcccggcctggtctcgagtcttttatagttctcactggcagctgtcaccagcaatttctctgagtgttgcccatctgcctggtctctctgacagggatgttcagaagctctcacctaaataaaagacccacctttcccagatatttgagggagagctcttgaagaagggaatgggatggcgggtgtggtggctcacacctgtaatcccagcactttgggaggctgaggtgggctgatccctcaaggtccggagttcaagaccagcctggccaacactgtgaaacctcctctctactaaaaaatacaaaaaattagctgggcatggtggtgggcacctgtagtcccagctacttgggaggcggaggcaggagagttgcttgaacccaggaggtggaggttgtggtgagcagagatcacgccactgcattccagcctgggaaacagagcaagactccgactcaaaaaaaaaaaaaatggggatatactggggccctggccctgctttgggtccatcccttctgccactaccatgcctaggaaccaggaggatttgggttctaacttcctgtgaagcaactcccttagagggccttttgccccatagaaggagctggcactgcttgtctgccagctctgccctcccagcatccagcaccccatctttattctggggctccagccctgtccctgtcctcaccttccttcctccttctcaccaaccaggcctctcttcacttcacctcacccctctgactatgttttcttctcctctccagtgaccaatgcactgcactccattctaggtgagtaggccacactgaagcggaagcggggagcggggaggaggccccaggctctggcagctgcctgaaactaagtcctcttcagtcaggaatgtagtaggtttaaaggcaggggtgggcagccgtggcaggtactggcttattgcccatggagggcccaggactggtgctccagtactgaacccctcacaccctgggtcccgacaggggagcagaccaaggcgctggtcacccaactcaccctcttcaaccagatcctggtggagctgcgggatgatatacgagaccaggtttgggtgggctggcgaagggtggcactgattctggggtagggtggcagatgtcaagtgctgactcctccccatccttctccaggtgaaggaaatgtccctgatccgaaacaccattatggagtgtcaggtgtgcggtgagtgggagagcaggggaggctccacatgaccgtgccacgttcccaccattggctttggctttcccttctggtggcttaaatagtgaccaccgggtagctctgactgtgtccacccctcaggcttccatgagcagcgttcccactgcagccccaatccctgcttccgaggtgtggactgcatggaagtgtacgagtacccaggctaccgctgtgggccctgcccccctggcctgcagggcaacggcacccactgcagtgacatcaatgaggtgagggaggtcagagcccagaagggtacagaaaactggggtgaggatgtcaggaggcacccaagagggtgggataaatgctggtccggaggagaggaatctggagtttaggagaggtcagaggcaagagaaatgcaagatgggagagacagaaggcctggggcaaagactgaaggccatacagggaaggagctggggaaactgcaggggaggcttgagatggcgaccagtggcatggggagggagagagggaaccccgagggaagtggggtggggaccaggagcacaaggcagttgtgtggggagagctgcacaaaggggagacctggagcaatggttcctggatcacaggcagggacctgagtttcccagagggcggcctgacctctgccttctcatctggtccccagtgtgctcacgctgacccctgtttcccgggctccagctgcatcaacaccatgcccggcttccactgtgaggcctgtcctcgagggtacaagggcacacaggtgtctggtgtgggcattgactatgcccgggccagcaaacaggtcagactgggtagtgtgtgtggacaagggatcttggccttgtagaggccaggggcttctgggttggacatgggacccttgtgatgaatgggacatggatggctttgcatcggctttgggtctgggcttaatgttgatgttcacagataaggccataccagtgtcctctgctgtatctgaggagacccaccataggtctggttccacccaaagtctgccccttcaggtctgcaatgacatcgatgaatgcaacgatggcaacaatggtggctgtgacccaaactccatctgcaccaacactgtggtgagctgaatatcctgagtgtattctggggtggtgggaatggtaaaaccccaacccccgccccttcttaccttcaaatttcctgctgcctttcctcctccccagggcactacacagattaggtgttcacatggacttggtttggaatactggtttgcctcttgctagctctttagcaggttatttaacttttctgagcctcagcttcctcatctgaaaactgaggctgttatccaccctgaaaggttgtcatgataacctaaaggagatactgatatgcctggcataactgagtgcccagcccacactccgcaggtggagcagtgctggtcctgggtgcctggtgggctcaccctccttcctaagatgctgcctctccactcttagggctctttcaagtgtggtccctgccgcctgggtttcctgggcaaccagagccagggctgcctcccagcccggacctgccacagcccagcccacagcccctgccacatccatgctcactgtctctttgaacgcaatggtgcagtgtcctgccaggtgagctaggcttcaggcgtggaaggaaaagggagggtctggggaaaggagtagggctatgtttagggcctgggttgggggtcttcataggagagaaggtgggcctgggcccaggaactgtttggtggggagaataggacctgaagcagggaaaatatagggaggaggggagccagaccaaactgctagctcctaccctttgtgttgccctagtgtaacgtgggctgggctgggaatgggaacgtgtgtgggactgacacagacatcgatggctacccagaccaagcactgccctgcatggacaacaacaaacactgcaaacaggtgcagggagcaggcgggcaagggggcgtagtggggagcccaagctgggtcaggccagaactcatccatcctcttcccctgaactttaggacaactgccttttgacacccaactctgggcaggaagatgctgataatgatggtgtgggggaccagtgtgatgatgatgctgatggggatgggatcaagaatgttgaggtgactcccagactgccctgccccttgaagctcctctcccctcctcctgtcctctctgtgcctcacttacctcatctggcagctctctagtaagggccaaatactccaaatcagggaggcaaaaacctcgtgcccaggacaaggaggctgggtgggtgggactgtactgagcagtttgtccatcacaagggtgtgatcttagaaaaggatacagagacaaggtaggtgcaagatgacaagtgatttaggggagacctgacccttcccctcccaccctctgcccaggacaactgccggctgttccccaacaaagaccagcagaactcagatacagattcatttggtgatgcctgtgacaattgccccaacgttcccaacaatgaccagaaggacacagatggcaatggggaaggagatgcctgtgacaacgacgtggatggggatggtgcaggcctggggctgaaggggtggctgggggacctgtgagaatttggatcaggtggggatgaagcagggaagctaggaagtctctgtgaaatagggaggcaggcttgtggacgttggcctgggtgaggagagattacctgcagcagatgtcaataggaatgtgaggtagggcgtagtgttaggcagagtgtggactagagggtgagacaagaaacaggcagatttcctggccagttgtcctctgggtggggagacaaagttcgggactttcaccaacctagaagagagaatatggcatgttctagtaacaaccttgtgctacccatgtcttctaggcatccccaatggattggacaattgccctaaagtccccaacccactacagacagacagggatgaggacggggtgggagatgcttgcgacagctgccctgaaatgagcaatcctacccaggtacagggagatggtaaggacaggggagggatgagggtactgatggatgaagccccagccctttggatggaaagtggtcagatcaccctcttcagagttatcaagaggagatggtgagaacaggtccctctctctcagacagatgcagacagcgacctggtgggggatgtctgtgatactaatgaagacaggtaaggtcttggtcaagagacgcaaggtctttcttttttttgtctttctgagacggcttgctctgtcacctaggctggagtacagtggcacgatcttggctcactgcaacctccgtctcccagggtcaagtgattctcatgcctcaacctccctgagtggctaggattacaggcatgtgctaccaagcccagctaattcttgtattttcagtagagacagggtttcaccatgttggccaggctggtctcgaactccttacctcaggtgatccgccagcctctgcctcccaaagtgctgtgattacaggtgtgagccactgtgccagcgaaatgcaaggtcttataggggatattttactttcctctagtatatccttttttttttttttttgagacggagtcttgctctgtcgcccaggctggagtacagtggcatgatctcggctcactgcaagctccacctcccgagttcacgccattcttctgcctcagcctcccaagtagctgggactacaggcgcctgccaccacgcccggctaattttttgtgtttttagtagagacggggtttcactgtgttaaccaggatggtctcgatctcctgatctcgtgatttgcctgcctcagcctcccaaagtgctgggattacaggcgtgagccaccgtgcccggcctcctttttttttttgagatggagtcttgctctgtcactcaagctggagtgcagtgatatcggctcactgcgacctccacctccagggttcaagcgattttcctgcctcagcctcccagtagctggattacaggcgcgtgctaccaagctcagctaatttttgtgtttttagtagagacagggtttcatcgtgttggccaggctggtctcgaactcctgacctcaggtgatctgcccgccttggcctcccaaagtactgggaatacaggcatgagccactgtgcccaggccatagtatattctaaattccttctatgatttagccttaattctctattgctattcagcaggaatttattcttacagtcatccctccattcctactgccagaccttcacctcacctggctgactgccaggggtctgattgtatggagagcaggctccagagactcccaggcagaagcgaagggaaggacaaggagtacctttaaatcctttatttggtacctgttcttctgattagcgatggggatgggcatcaggacaccaaggacaactgcccacagctgccaaatagctcccagctggactctgataacgatggacttggagatgagtgtgatggggatgatgacaatgatggcatcccagattatgtgcctcctggtcccgataactgccgcctggtacccaatcccaatcagaaggactcagatggtaagcctgcggacccagagcacgttagactggtgttgcctttgcccaggtggaggcagcaagccctgttgggaagtgaggaagggcaaggtgggaaagatgtcaggaatgcaggcccaacagatggtattgttgcctaatggcagtggccagggccttcctgagcacccagcctcactctgcccaggcaatggcgttggtgatgtgtgtgaggatgactttgacaatgatgctgtggtcgaccccctggatgtgtgtcctgaaagtgcagaggtaacgcttacggattttcgggcctatcagaccgtcgtcctggatcc

SEQ ID NO: 2 - DNA, 1500 bp, Homo sapien
atgccgcatcctcgaaggtaccactcctcagagcgaggcagccgggggagttaccgtgaacactatcggagccgaaagcataagcgacgaagaagtcgctcctggtcaagtagtagtgaccggacacgacggcgtcggcgagaggacagctaccatgtccgttctcgaagcagttatgatgatcgttcgtccgaccggagggtgtatgaccggcgatactgtggcagctacagacgcaacgattatagccgggatcggggagatgcctactatgacacagactatcggcattcctatgaatatcagcgggagaacagcagttaccgcagccagcgcagcagccggaggaagcacagacggcggaggaggcgcagccggacatttagccgctcatcttcgcagcacagcagccggagagccaagagtgtagaggacgacgctgagggccacctcatctaccacgtcggggactggctacaagagcgatatgaaatcgttagcaccttaggagaggggaccttcggccgagttgtacaatgtgttgaccatcgcaggggtggggctcgagttgccctgaagatcattaagaatgtggagaagtacaaggaagcagctcgacttgagatcaacgtgctagagaaaatcaatgagaaagaccctgacaacaagaacctctgtgtccagatgtttgactggtttgactaccatggccacatgtgtatctcctttgagcttctgggccttagcaccttcgatttcctcaaagacaacaactacctgccctaccccatccaccaagtgcgccacatggccttccagctgtgccaggctgtcaagttcctccatgataacaagctgacacatacagacctcaagcctgaaaatattctgtttgtgaattcagactatgagctcacctacaacctagagaagaagcgagatgagcgcagtgtgaagagcacagctgtgcgggtggtagactttggcagtgccacctttgaccatgagcaccatagcaccattgtctccactcgccattaccgagcaccagaagtcatccttgagttgggctggtcacagccttgtgatgtgtggagtataggctgcatcatctttgaatactatgtgggattcaccctcttccagacccatgacaacagagagcatctagccatgatggaaaggatcttgggtcctatcccttcccggatgatccgaaagacaagaaagcagaaatatttttaccggggtcgcctggattgggatgagaacacatcagctgggcgctatgttcgtgagaactgcaaaccgctgcggcggtatctgacctcagaggcagaggaacaccaccagctcttcgatctgattgaaagcatgctagagtatgaaccagctaagcggctgaccttgggtgaagcccttcagcatcctttcttcgcccgccttcgggctgagccgcccaacaagttgtgggactccagtcgggatatcagtcggtga

SEQ ID NO: 3 - protein, 499 aa, Homo sapien
MPHPRRYHSSERGSRGSYREHYRSRKHKRRRSRSWSSSSDRTRRRRREDSYHVRSRSSYDDRSSDRRVYDRRYCGSYRRNDYSRDRGDAYYDTDYRHSYEYQRENSSYRSQRSSRRKHRRRRRRSRTFSRSSSQHSSRRAKSVEDDAEGHLIYHVGDWLQERYEIVSTLGEGTFGRVVQCVDHRRGGARVALKIIKNVEKYKEAARLEINVLEKINEKDPDNKNLCVQMFDWFDYHGHMCISFELLGLSTFDFLKDNNYLPYPIHQVRHMAFQLCQAVKFLHDNKLTHTDLKPENILFVNSDYELTYNLEKKRDERSVKSTAVRVVDFGSATFDHEHHSTIVSTRHYRAPEVILELGWSQPCDVWSIGCIIFEYYVGFTLFQTHDNREHLAMMERILGPIPSRMIRKTRKQKYFYRGRLDWDENTSAGRYVRENCKPLRRYLTSEAEEHHQLFDLIESMLEYEPAKRLTLGEALQHPFFARLRAEPPNKLWDSSRDISR

SEQ ID NO: 4 - DNA, 1044 bp, Homo sapien
atggctcagagcagagacggcggaaacccgttcgccgagcccagcgagcttgacaacccctttcaggacccagctgtgatccagcaccgacccagccggcagtatgccacgcgtgacgtctacaacccttttgagacccgggagccaccaccagcctatgagcctccagcccctgccccattgcctccaccctcagctccctccttgcagccctcgagaaagctcagccccacagaacctaagaactatggctcatacagcactcaggcctcagctgcagcagccacagctgagctgctgaagaaacaggaggagctcaaccggaaggcagaggagttggaccgaagggagcgagagctgcagcatgctgccctggggggcacagctactcgacagaacaattggccccctctaccttctttttgtccagttcagccctgctttttccaggacatctccatggagatcccccaagaatttcagaagactgtatccaccatgtactacctctggatgtgcagcacgctggctcttctcctgaacttcctcgcctgcctggccagcttctgtgtggaaaccaacaatggcgcaggctttgggctttctatcctctgggtcctccttttcactccctgctcctttgtctgctggtaccgccccatgtataaggctttccggagtgacagttcattcaatttcttcgctttcttcttcaatttcttcgaccaggatgtgctctttgtcctccaggccattggtatcccaggttggggattcagtggctggatctctgctctggtggtgccgaagggcaacacagcagtatccgtgctcatgctgctggtcgccctgctcttcactggcattgctgtgctaggaattgtcatgctgaaacggatccactccttataccgccgcacaggtgccagctttcagaaggcccagcaagaatttgctgctggtgtcttctccaaccctgcggtgcgaacccgagctgccaatgcagccgctggggctgctgaaaatgccttccgggccccgtga

SEQ ID NO: 5 - protein, 347 aa, Homo sapien [sequence omitted]

SEQ ID NO: 6 - DNA, 2010 bp, Homo sapien
atgatgccctcgcctagtgactccagccgctcgctgaccagccggcccagcaccaggggccttacccacctccgcctccaccgaccctggctgcaggccctgcttacgctggggctggtccaagtgctcctgggcatcctggtggtcaccttcagcatggtggcctcttccgtcaccaccaccgagagcatcaagaggtcctgcccgtcttgggctgggttctcgctggcgttctccggggtggttggcattgtgtcctggaagcggccattcactctagtgatctccttcttctccttgctttcggtgctctgtgtcatgcttagcatggctggctctgttctctcctgtaagaatgctcaactggcccgagacttccaacagtgctctctggaaggaaaggtctgtgtgtgctgtccctctgttcccctcctccggccctgtccagagtcggggcaggaactgaaagttgcccctaactccacctgtgatgaagcccgaggggccctcaagaacctgctcttcagcgtctgtgggctcaccatttgtgccgctataatctgtacactctctgctattgtctgctgcatccaaatcttctccctggacctcgtgcatacgcagctggcccctgagcggtcagtctcaggcccactgggacctctgggctgcacgtccccgcccccagcccctctcctacacaccatgctggacctggaggaatttgtcccgcctgtgcccccaccgccctactatcccccagagtatacctgcagctcagaaacagatgcacagagcatcacgtacaatggctccatggacagcccagtgcccttgtaccctaccgattgccccccttcttatgaggcagtcatgggactacgaggagacagccaggccactctctttgaccctcagcttcacgatggctcgtgcatctgtgaacgagtggcctccattgtagacgtgtccatggacagcgggtctctggtgctgtcagccattggtgacctccctgggggctctagcccgtcggaggactcgtgcctgctggagctgcagggctccgtgcgctccgtggactacgttctctttcgctccatccagcgcagccgtgccggctactgcctcagcctggactgtggcctgcggggccccttcgaggaaagccccctgccacggcgccccccacgggctgcccgctcctattcctgctctgcccctgaagctccacccccactgggtgcccccacagctgcccgcagctgccaccggttggagggctggccgccctgggtgggaccctgcttccccgagctgaggcggcgggtcccccggggagggggccgcccagccgcagccccgcccacccgagccccgactcgtcgcttcagcgatagctcaggttccctcaccccaccggggcaccggcctcctcatccggcatccccaccaccgctgctgctgccacggtcccacagcgacccaggcatcacgacctccagtgacactgctgacttcagggacctttataccaaagtgcttgaggaagaagctgcttctgtttcctctgcagatacagggctctgctctgaagcctgcctcttccgcctagcccgctgcccttcccccaagttgctacgtgcccggtcagccgagaaacggcgccctgtgcccaccttccaaaaagttcccctgccctcgggccctgcacctgcccactccctgggggacctaaagggcagctggccaggtcggggcctggtcactcgtttcctccagatatccaggaaagccccagaccccagtgggactggagctcatggacataagcaggtgccccggagcctgtggggccggcctggccgagagagcctccaccttcgcagctgcggagatctgagctctagctcttccctgcggcgtctcctgtctggccgcaggctggagcgtggtacccgcccccacagcctcagcctcaacgggggcagccgggagactgggctctga

SEQ ID NO: 7 - protein, 669 aa, Homo sapien
MMPSPSDSSRSLTSRPSTRGLTHLRLHRPWLQALLTLGLVQVLLGILVVTFSMVASSVTTTESIKRSCPSWAGFSLAFSGVVGIVSWKRPFTLVISFFSLLSVLCVMLSMAGSVLSCKNAQLARDFQQCSLEGKVCVCCPSVPLLRPCPESGQELKVAPNSTCDEARGALKNLLFSVCGLTICAAIICTLSAIVCCIQIFSLDLVHTQLAPERSVSGPLGPLGCTSPPPAPLLHTMLDLEEFVPPVPPPPYYPPEYTCSSETDAQSITYNGSMDSPVPLYPTDCPPSYEAVMGLRGDSQATLFDPQLHDGSCICERVASIVDVSMDSGSLVLSAIGDLPGGSSPSEDSCLLELQGSVRSVDYVLFRSIQRSRAGYCLSLDCGLRGPFEESPLPRRPPRAARSYSCSAPEAPPPLGAPTAARSCHRLEGWPPWVGPCFPELRRRVPRGGGRPAAAPPTRAPTRRFSDSSGSLTPPGHRPPHPASPPPLLLPRSHSDPGITTSSDTADFRDLYTKVLEEEAASVSSADTGLCSEACLFRLARCPSPKLLRARSAEKRRPVPTFQKVPLPSGPAPAHSLGDLKGSWPGRGLVTRFLQISRKAPDPSGTGAHGHKQVPRSLWGRPGRESLHLRSCGDLSSSSSLRRLLSGRRLERGTRPHSLSLNGGSRETGL

SEQ ID NO: 8 - DNA, 6439 bp, Homo sapien
ggtagttggttgtgggcactgggttagaggtatcacgtgggggcactttcgtcttagcttttggacaagacgcaggcgcaaacccacggctgctgcgggggatccttgtggccctttccggtcggtggaaccaatccgtgcaacagagaagcggggcgaactgaggcgagtgaagtggactctgagggctaccgctaccgccactgctgcggcaggggcgtggagggcagagggccgcggaggccgcagttgcaaacatggctcagagcagagacggcggaaacccgttcgccgagcccagcgagcttgacaacccctttcaggtgacttgcgccagtcggcctcttttgggcggtcaggttgattcttcccggttctgtagggtcgggctaacttgtatccccatttgtgacatttgatcctgggaagagccgccacgtggggtgacagtgactccagaccagtgagcactctggggggcgggccctgccccttaatgggctggtgctggcagtgttggatgatggatttgaggtaaatgttgtcccagtcctgggacaacggcgtggccccgaaggtgcagtacttggagccacaggcagtttgggaatggtccctgggaattcccctaggtggcactgggtgccagctgagacccgggtctctgccctcaggacccagctgtgatccagcaccgacccagccggcagtatgccacgcgtgacgtctacaacccttttgagacccgggaggtgagctactggagagcataggagttcaaggaagggaagggtttgccagtgagacaagttagcttgggtacaggggacttttctgcatgcacaggaaggaagaggcactgtgatcttagttccctgagagaggctagtcagccatgggatgcctcatccttctagggccaccagggccaccaacccaaatggtttgagctctaaaagtcagtcctgggaagatctggaggctggcaaggccttaaccaatgttctgtatgggaaggctttcttgaagaaaataaaggttggggctggttggagtttaggttaggtgtgcagtttgaaagcaaggaatgaggaggactacacctgtttgtcccactggattagaagatggtgagttgtaagaatttaaggggttttcatggtacttaaaacttaaaaaaaaaagccaagcatggtgtgcatgcctgtaatcccacttgggaggctgaggtgggaggattgcttgagcccaggaatttgatggagaccatcctgggcaacagcgagacccccatctcttgagacgttttaaaaattggctaggtgtagtggcacatgcctgtagtccttgctacttgggaggctgggatggaaagactgcttgggcccaggagtttgaggcttcgatgagtgatgattgcaccactgcactccagcctgggtggcagagagacaaccccacactccatccccagaagaaaagagaaaaaaaaaacacagttaagtggggagatatggtaaatgagtgatttgagtcctcactaaggaattggatatgagggatgataaggctgctacaggtcatcatagagttctctgacagaaattcaacagaagaactagcttcctaacaaatgggggaaaggctgcttccttacatcatgagttctgggtctaaaggtattcaagcagactatagattcctgtgttatgagggactagatgatcactaaggtcttttcctcaacagccaccaccagcctatgagcctccagcccctgccccattgcctccaccctcagctccctccttgcagccctcgagaaagctcagccccacagaacctaagaactatggctcatacagcactcaggtacaggaggtgtgcaggtgagggctggggagaagggccagtcctggggcccagggctcacatctccgtctgctcacactgggcaggcctcagctgcagcagccacagctgagctgctgaagaaacaggaggagctcaaccggaaggcagaggagttggaccgaagggagcgagagctgcagcatgctgccctggggggcacagctagtaagtaatagagtggggaagagcatctgagagtttgggaggagcaaaaacaggtcttaagggcctgggctcggctgggtgcggtggctcacacctataatcccagcactttgggaggctgaggtgggcggatcacgaggtcacgaggtcaagaaatcgagaccatcctagccaacatggtgaaaccctgtgtctactaaaaatacacacacacaaaaattagctgggcatggtggcgcgcacctgtagtcccagctactcgggaggctgaggcagggagaatcgcttgaacccgggaggtggaggttgcagtgatccgaaatacgcgccactgcactccagcctggtgacagaaatgagactccatctcaaaaaaaaaaaaacacaaacctgggctctggagtcagactgctgagtttgaattttagagatactgcttactaaccatgagcttttagggaagttacttaatcttgaatgcctcagtttccctatttataaaatgaaaaccacgtttgcatccgtttgacagggttgttgtgaaggttaaacaaaatatatgtgaagtacttggtacagtgcttagatgttttaaataataataaacagtattaatttttccactctcacttgtctccttggaccccaaattggagataaaagagaggatccagggctgggtatggtgcctcacgcctgtaattccaacactttgggaggccgaggcaggcaggttgcttgagttcaggagttagagaccaacctggacaacatagtgagatcctgcctccaaaacattaatgaaaaaaattagcagggcatgatgtacctgtctgtagtcctagctcctcaagaagctgaggtggacggccaggcgccggggctcatgcctgtaatcccagcactctgggaggccaaggcgggtggatcacctgaggtcaggagttcaggaccagccaacatggtgaaaccctgtctctactaaaaatacaaaaattagctgggcgtggtggcatgtgcccataatcccagctactcaggagactgaggcaggagaattacttgaacccgggaggcggagattgcactgagccaagatcacaccactgcactccaacctgggcaacaagagcgaaactctatctcaggagaaaaaaaaaaaaaaaaaaaagctgaggtgggagggttgcttgagcccaggaggttgaggctacagtgaaccatgatcataccactaccttccagcctgaacaacagagaccctatctcaaaaaaaaaaaaaaaaaaaaaagagaggatccagggatggagagaagggggagtgattcctttgttggtctctgttttacttctggggtccacctgttttttggcgccttacagctcgacagaacaattggccccctctaccttctttttgtccagttcagccctgctttttccaggacatctccatggagatcccccaagaatttcagaagactgtatccaccatgtactacctctggatgtgtgagtagtgagaagccttttggaggaagttacaggtagatctcttaactgccctggggtccgctcaccaagaaccaaacacttcacctctatttagaactcaccagcctgctagcaaatgttcttgccctctccccactttttattgtccatatgcaggaatatattttgaattctttagatgtctttgggctgggtgcagtggtatacgcctgtaatcccagcactttgggaagctgaggtgggtggataacctgaggtcagaagtttgagactagcctgatcaacatggagaaaccccatctctactaaaaatacaaaattacctgggcgtggtggcacatgcctgtaatcctagctactcaggaggctgaagcaggagaatcacttgaacccgggaagtggaggttgcaatgagccaagatcatgccattgcactccagcctgggcaacaagagcaaaactccatctcaaaaaaaaaaaaaaaaaaaaaaaaaaaaaaaaaaaggccgggcacggtggctcacacctgtaatcccagcactttgggaggcccaggtgggcagaacatgaggttaggagatcaacaccatcctggctaacacggtgaaaccccgtctctactacaaatacaaaaaattagccgggcgtggtggcgggtgcctgtagtcccagctacttaggaggctgaggtaggagaatgggcgtgaacccaggaggcagagcttgcagtgagccgagatcctgccactgcactccagcctgggcgacagagcgagattccatctcaaaaaaaaaaaaagaaaaaaaaaaattagctgggcgtggtggcgggcgcctgtagtcccagctactctggaggctgaggcaggagaatggcgtgaaccggggaggcggagcttgcagtaagctgagattgcgccactgcactccagcctgcgcgacagagccagactccgtctcaaaaaaaaaaatgtctttgcaaggggatcacacattattgacatttgctttctccatctcccctgttgaggattccatgaaggcagcagctgccttcatttccttactctgccatgtttggtgaatattataggatgagcatagatgggaaggagccttcattgcagtccagaagggctcctcatcctgtccctctgccccttaggcagcacgctggctcttctcctgaacttcctcgcctgcctggccagcttctgtgtggaaaccaacaatggcgcaggctttgggctttctatcctctgggtcctccttttcactccctgctcctttgtctgctggtaccgccccatgtataaggctttccggtaagtgtgttagtggtgggagagtgatggagacctgggatgggccccacgtctgcccatccttcagctctaattcttctcccaccctccccatttttttcctctttgtaggagtgacagttcattcaatttcttcgctttcttcttcaatttcttcgaccaggatgtgctctttgtcctccaggccattggtatcccaggttggggattcaggtttgtgaggctgttatccaccctcacctttccctctagatccagccagcactgggtgctggataggagttgttcagaaaaaggaaatgtggttttaatccttgggaggtactagtttaatgagatacaagacatacttccaggatagagcgcagacagcactcttgacacatatagattgaagggaagaatgctgcatttggccaatctaaggtggcttcctggaggaggcatagaaccatggttgaaaggaggaagaagaatccccagatgagtgcctggaagagcttggagagctcagggctaatggttcagaaactggaattaaactatgaagagattagatacagtttgggattgtggtgcttggaatgctgctcgattgacagggagccactgctgatgggaagggtaggaacaggggatgctggtgacataaccagtggagaagctgaggagcccctcttcactggtacatccttccctttacagtggctggatctctgctctggtggtgccgaagggcaacacagcagtatccgtgctcatgctgctggtcgccctgctcttcactggcattgctgtgctaggaattgtcatgctgaaacgggtgagggctgtgtcgaaggtggggccgggatggtgagatcatgggtccccaggggcgtgggtggaacattcaggagcaactggcacaggtcaggctgctgggttgttctcagctaatggacctctggggtgtgtgtttctgtgtgtgagtgtgtgtgctgggcagcaggctgctgagtggtagtgatgctgttaggctgggggtggggaaccagtggctggaatgggcggtaatgtctttgtcctctacttgcagatccactccttataccgccgcacaggtgccagctttcagaaggcccagcaagaatttgctgctggtgtcttctccaaccctgcggtgcgaacccgagctgccaatgcagccgctggggctgctgaaaatgccttccgggccccgtgacccctgactgggatgccctggccctgctacttgagggagctgacttagctcccgtccctaaggtctctgggacttggagagacatcactaactgatggctcctccgtagtgctcccaatcctatggccatgactgctgaacctgacaggcgtgtggggagttcactgtgacctagtccccccatcaggccacactgctgccacctctcacacgccccaacccagcttccctctgctgtgccacggctgttgcttcggttatttaaataaaaagaaagaggaactggaactgacat

SEQ ID NO: 9 - DNA, 8253 bp, Homo sapien
cctgcggcagccggagctcggggagcggagcgtggtggggaggggagcgggacaggcgacacaggagacagcggcgccgcggcctctccccaccaggcggccccggatcctactggacgccctgagggcacaccgaccgcgcctctagagtcaccccacgccgacccctcccctcttctctagacttatttccatccttcccgcttttaccctccccacccgtccctgggctccaggccgccgccccctcctcactcctggaccggcccttctcggtgcccctcttccctagggagatgcgatgagccggtgcccccgcgtcctcatcgtcgccccgggcacggtgcccgtccagtgcccgtggtggggagggagcactccgcggtccctccgtgacgcccctcgcttggccccccccacagctggcgtccctcggccatgccccaggggacccagccagggggtgggctctagagcgagtggggtggagaggagaaaggacggggccttgggggcctctgagatgctcccaagtgccagggagggccgagcgaggcgcaggcaaccgggcagcaggcatgatgccctcgcctagtgactccagccgctcgctgaccagccggcccagcaccaggggccttacccacctccgcctccaccgaccctggctgcaggccctgcttacgctggggctggtccaagtgctcctgggcatcctggtggtcaccttcagcatggtggcctcttccgtcaccaccaccgagagcatcaagaggtcctgcccgtcttgggctgggttctcggtgagttgggtgcacagttgttgggtgggggaggctcctcgggccccaccctccacaccagctgcaagtccacatgcttgcctctcctccctctcctggtcctctgcctccttacagggctgggtgcatcctgtggtgaggggctcactcaggagcctccccctgccaggctgagcctgtgcccactctgtccccagctggcgttctccggggtggttggcattgtgtcctggaagcggccattcactctagtggtaggtgccagggtccagtgcccactgggaggcaggtgcccagcacgcaaggggaagcccatttatgacctcaagaagggaactggctccccagttggtgccagccgggtgggcacgaagttctgtgaaggagggggactctgtccgtggcagaggagtactcatgagggtcccagccctgagtctgcaccctgttttccccagatctccttcttctccttgctttcggtgctctgtgtcatgcttagcatggctggctctgttctctcctgtaagaatgctcaactggcccgagacttccaacagtgctctctggtgagatttgaggagggagagctggaaagaactggctgggggaggtgtgcaggacacctcagtttgtgctgactcaggctgcctcaccctccctgctccactcaggaaggaaaggtctgtgtgtgctgtccctctgttcccctcctccggccctgtccagagtcggggcaggaactgaaagttgcccctaactccacctgtgatgaagcccgaggggccctcaaggtgagcttgcaccctgcaaacatcctcctggttctcactcttgcctcccctgctggggatctcacaggcccataatgtgtggaacttagccgtctcctgactcctctcctcctttcccttccccagaacctgctcttcagcgtctgtgggctcaccatttgtgccgctataatctgtacactctctgctattgtctgctgcatccaaatcttctccctggacctcgtgcatacggtgagaagggagcaggggccagggcacgcaggtatggtgggggcaggggtgtgtgtgctgagacttgcctgagggaacaatggctacagatctggcttttgagcaccaggggctatgggttatctattatcctcatctattggaggaatggatgtttagtggggaggatgagaaggggagatggcagggagtgagttaagtatgtgatgctggtctgggaccaggagagggtgctttagagaatgggactggatggtggggtagagtcaagaaggtcctatgctgggcacggtggctcattcctgtaatcccagcactttgagaggcccaggcaggcggatcacctgaggccaggagttcaagacccacctggccaacatggcgaaaccctgtctctattaaaaatacaaaaatcgggccgggcgcagtggctcgtgcctataatcccagcactttgggaggctgaggagggcagatcacctgagatcaggagatcgagactatcctggctaacatggtgaaaccccatctctactaaacatacaaaaaattagccaggcgtggtggcgggcgcctgtggtcccagctactcgggaggctgaggcaggagaatggcatgaacctgggaggcagagcttgcagtgagctgagatagtgccactgcactccagcctgggcgacagagcaagactccatctcaaaaaacaaacaaacgaaatacaaaaattagccaagtgtggtggcgggtgcctgtaatcccagctacttgggagtctgaggcaggaaaattgcttgaaccggggagtcagaggctgcagtgagctgagatggtgccactgcattccagcctgggcgacaagagcacagactccgtctcaaaaaaaaaaaaaaaaaaaaaaaaaaaatatatatatatatatatatatatatatatatacacacaaaattacaaaaattaggctgggctccgtggctcatgcctgtaatcccagcactttgggaggccaaggcaggaggatcaccagatatcaggagtttgacaccagcctggccaacatggcgaaaccctatctctactaaaaatacaaaaattatccgggtgtggtggcgggtgcctgtaatcccagctactcggaagactgaggctggagaatcgcttgaacctgggaggcagaggttgcagtgagctgagatgtagccattgtactccagcctgggcgacaagagtgaaacttcgtctcgaaataataataataataataattagctgggcatggttgcacacccttataattcctgctactcaggaggatgaggcatgggaattgcttgatcttgggaggtgggggttgcagtgagctgagatcgcgccactgcactccagcaacagagtcagactctgtctcaaaaaaaaaaagaaggtcctagatggaggtgaggctaaagggtggacattcctgtgaagacacagaatgtgtggagcttctggatggaggtggggccataaagaggaatttgtgggtgggcagggccagaggctagaggtacagggtgacggtggggttaaggagagctgattttgggtgagggaggggccagaataagagcttctctcaggggatagggccagaaaaaataatgtagatgagagaggagtttggggcccaggaggagctgtattcccagaatccagacttgctgacctgctcgcttccccagcagctggcccctgagcggtcagtctcaggcccactgggacctctgggctgcacgtccccgcccccagcccctctcctacacaccatgctggacctggaggaatttgtcccgcctgtgcccccaccgccctactatcccccagagtatacctgcagctcagaaacagatgcacagaggtaaggcctggtggggtcttgctgggggagctaaggaaggggactggggctgggcctggattgctggagagagggatctttgtggagggggaattatttctcctacattgaccctcttcctcatctgccagcatcacgtacaatggctccatggacagcccagtgcccttgtaccctaccgattgccccccttcttatgaggcagtcatgggactacgaggagacagccaggtgagagcacagcacggcttggggcgggctggggagccgggttgtagcctggaaagctgaacaggctgtagcctctggataaagatagtaacagtggtcaaggtctttacagcaccagcatgcccactgttgcctttgatcttccctagagcctggtgaggtgagtgggttggaagctatcatttctgccatacagacgtggaagctgaggctgcacaattaagtgacttgtacaaagggacaaggctggtctggaatctaagtctccagagctctgtctagcacatgggggtgttcagtgtgtagggctgaacccttgaccctgtgtcttctgcaggccactctctttgaccctcagcttcacgatggctcgtgcatctgtgaacgagtggcctccattgtagacggtgagcagggcgtaatgaggggtggacaagggcggggctgccagggatagctggggtgggtagagacaatagaaggggaaaacaaggcggagttggtggtttggggacataggagggctgtggcaacttggaaccctggacttatttttctcctctgagataaagctggaggcacagtggcccctagaggctgggcttgggagaagaggaactgcctgggcagggctaggccaggccaggctggtgctacacagcgccccctgccgcccacagtgtccatggacagcgggtctctggtgctgtcagccattggtgacctccctgggggctctagcccgtcggaggactcgtgcctgctggagctgcagggctccgtgcgctccgtggactacgttctctttcgctccatccagcgcagccgtgccggctactgcctcagcctggactgtggcctgcggggccccttcgaggaaagccccctgccacggcgccccccacgggctgcccgctcctattcctgctctgcccctgaagctccacccccactgggtgcccccacagctgcccgcagctgccaccggttggagggctggccgccctgggtgggaccctgcttccccgagctgaggcggcgggtcccccggggagggggccgcccagccgcagccccgcccacccgagccccgactcgtcgcttcagcgatagctcaggttccctcaccccaccggggcaccggcctcctcatccggcatccccaccaccgctgctgctgccacggtcccacagcgacccaggcatcacgacctccagtgacactggtgagccccctccccgactgcccaggctcaggagagggtaggcactgggagttaggtggccagtgatgcccaccaggattggggcacagttgaggtgggtgaggaggaagaaagggtgagttcactcctctggtagacattctcaggtactcacctcctagggacagaacatccatagcttagcagctaaaaaaggagaatttttttttttttttgagacggagtctctggctctgtcgcccaggctggagtgcagtggcatgatcttggctcactgcaacccgtttcccgggttcaagtgactctctcctgcttcagcctgccgagtagctgggactacaggtgtgcgccaccatgaccggctaatatttttttttttttttttttttgagacggtgtctcgctctgtcacccaggctggagtgcagccgcgcgatctgggctcactgcaactccgcctcccgggttcacgccattctcccccgggttcacgccattctccttcctcagcctcccgagtagtgagtagctgggactacagatgcctgccaccacgcccggctattttttgtatttttagtagagatggggtttcaccgtgttatccaggatggtctcaatctcctgacctcgtgatctgcccgcctcggcctcccaaagtgctgggattacaggcatgagtcaccgtgcccgggcaatttttgtattttttagtagagacaggtttcacccttttggccaggctggtcttgaactcctgacctcaagtgatccacccgcctcggcctcccaaaaggatttattttttgaaaccagttccacagctctcagcttggtccacttatctgtcctccccaagcttcagctgtcacttgttaacatgtataataatagtacttcacgccgggcacggtggcttgcacctgtaatcccagcactgtgggaagctgaggtgggtggatcacctaggtcgggagttcgagaccagcctggctaacatggtgaaaccctatctctactaaaaatacaaaaattagccaggtgtggtggagcgcgcctgtaattccagctactaacgagagaggctaaggcaggagaatcgcttgaacctggaaagcaggggttgccgtgagccaagatcatgccactgcactccagcctgggtgacagagacacactccatctcaaaaacacaaacaaacaaacaaaaaacatgtataataacagtacttcagccataggcattgtactcgaagatgctgagaaagaacagtggcgggcaaggctgttcacacctataatcccagcactttggaggccaaggcaggtggatcacctgaggtcaggagttcaagaccagcctagccaacatggtgaaacccccatctctactaaaaattgaaaaattagctgggcctggtggtggacgcctgtaatcccagctactagggaggctgaggcaggagaatcgcttgaacctgggaggcggaggttgcagtgagctgagatcgtaccactgcactccggcctgggcaacacagtgagactccatctcaaaaaaataagaaaggagatagtactggggaacgctcagcactgtgcgccaggtgctgaacaacaccactgcagtccttgttgtggtggattgtaccatctagttgctggctaatatggacagagatgctggccctttgattggggatggagcgtgggagctgtgaaagctcctctgggcttgagttcccacaggagggtgggcgtgtccacagaacacttccactcactccctgtctccctttctctcttctccccagctgacttcagggacctttataccaaagtgcttgaggaagaagctgcttctgtttcctctgcagatacaggtcaggcatgtggtttgcgccccagggatggggattgggcatggctgcccagccccctctccaccctacaataccattctcttatctctgtctctctgcagggctctgctctgaagcctgcctcttccgcctagcccgctgcccttcccccaagttgctacgtgcccggtcagccgagaaacggcgccctgtgcccaccttccaaaaagttcccctgccctcgggccctgcacctgcccactccctgggggacctaaagggcagctggccaggtcggggcctggtcactcgtttcctccagatatccaggaaagccccagaccccagtgggactggagctcatggacataagcaggtaggaattcggggagccaggaaagatgtttgggaaagcgtggagcttcagattgagccttattgatgatgccctttcttgtgtccctgtccaggtgccccggagcctgtggggccggcctggccgagagagcctccaccttcgcagctgcggagatctgagctctagctcttccctgcggcgtctcctgtctggccgcaggctggagcgtggtacccgcccccacagcctcagcctcaacgggggcagccgggagactgggctctgacctaggcttcttgtcacactgaacacatccagccacaggcaccagctggttgggaccagcagcccccagcatcctcttgcactggctggcacaaaaagaaacctgctgtataccccccaaagtgtccctttccctcctacctctggggtctcttgctgcttgcctctgctgctctggtctgggagagcttctgtcctgtgctgcatgggtatttagactgtgggggagatgccccttcttatagcactggaggaggaaaacaaattcttgtccccctcagaatgagagtggctctttctgatttgcaagggcactatggtcagggcaaaggcatggcccaggtgtttaagtacagggtgacgtgtgcctatgcaatggggtggtaaggcaggcacgaagagtccaaaaaatctaggtggcctctcagctctgccacctctagctgcatgaccttgggcaagctatgtaaccccaattgcctgctccattaaagactgtgaaggtagaatgtttgtaaagctcttaacagtatgtaagccttcaataaatttcagttttcccctt

SEQ ID NO: 10 - DNA, 10482 bp, Homo sapien
tcccagggtcccgggttgggggggtggagcagcatttcgtcgccgcgggggtgccgggactccggccgcagtgtcgccgccatcacggacttcctgtgggacaagcgcacgggcctcgccgccagaacggtgagcgcgcgggttggctggccgcgcgaaaagatggcgaccgcggggcgggcggggttaggcttgggttgggggcaagggacgggggcgagatttggagaggaggagagttgagacctacgaagcgacggagtagggagatgagggggaaggaggtcggcctgcgttagatgtaccaagaagacctgtaggaagcttgggcggaaccagactagaagggatgatagagtaaggatgggagtggttccagagatgtgaaggagtggaatatcaaagttggactcgggtccaatcaaagctggaggggaggaatacatcaccgtgtcacttggaacatgcaggaagaatttgcctgtaggagttctggattttctgagtaagaaaggaaaaagcaaaagatttcaaacttgagaaaggtaggctagtgatgttttgaggggacagtgctgtgaggccgtttgatggtagtatgtggtgagatggcatttatgtggctatgctgattaggataactatgctgggaaaatcttgtgcttgcagcttggtatgctaccggccccagttacaccaagaatgcatcaaataccaccctcctcaatttggggaggttgggaagaagtgtatacagaatggtggtcagacatcggaatcattaaaccttttgcatacgcgattctcatacacccccaccaagacttatgtccctgggcctgtttcattttattgtgccttcctattctcatcttcctggattgtgacagtgatttttctcttttttccttgaaggaaaaaaaggatttgtgaaccatcccattccaattccattaactggaaggtggccagctgtcagctaagtagggctgatattaattaaaaaccacttactggccaggcgcggtggctcacatctgtaatcccagcactttgggaggccaaggcgggcggatcacctgaggttgggagttcgagaccagcctgaccaacatggagaaaccctgtctctactaaaaatacaaaattagctgggcgtggcggcgcatgcctgtaatcccagctactcgggaggctgaggcaggagaatcgcttgaaccctgcaggcggaggttatggtgagccgagattgcgccattgcactccagcctgggcaacaagagcaaaactccgttttcaaaaaaaaaaaaaaaaacacaagaaacagttgttatctacgtcttaagcacaagagtggaggggccagatgggatgctgactgtattttgtggcctactacattaaaacctctcaagggagagttggccaaagatgtgagagatgattaagttaattcaaggggcccgacccagatccctggagctgatagtatttggcgtttacctggtatatgtagaaacctgggagactggattcctaccctcaggacactgatagtctacttgaggagatgagatgtactcttaatgagtgtacaacatttatcataactgtaaaccaaagctttactctattgacttgtgaggaactcagcatcacacagatctatatcttagatcctgaacggggatacactgtggcagtctccatcggtcgtcatacactttgctctgtagtgaactgtgtcctagacctgaaggtcttcatggagctgtgtttgaagaaaataaggatagaacacttgaactggctgggtgcggtggctcacgcctgtaatcccagcactttgggaggccgaggcaggaggatcacgagtcaggagtttgagaccatcctggccaacatggtgaaaccccgtctctactaaaaatataaaaattagccaggagtggtggtgcgtgcctgtaatcccagctacacaggaggctgaggcaggagagttgcttgaacctgggaagtggaggttgcagtgagccgagattgtgccactgcactccagcctggccacacagcgagactctgtctcaagaataaaataagaacacttgaactgaggggtgggtaatgagggatgggtttgggggctttctagagtttcagccagagttagtaggtggctgtactccaataaggaggaattgtaagtacagaggaagactgaggaagttgtgcctggtaaaaagaggcagaaataagaggggaaagcagcttagcatctttggagcagccagtccttggaggagatttctgaaaactcgaaactttcctatttccgctttcccttgccttccagatgccgcatcctcgaaggtaccactcctcagagcgaggcagccgggggagttaccgtgaacactatcggagccgaaagcataagcgacgaagaagtcgctcctggtcaagtagtagtgaccggacacgacggcgtcggcgagaggacagctaccatgtccgttctcgaaggtgaggcctcaggaagagatactcaaacacagggtcactcgctcattcctttgttcaacaaatacttattggttatatgctaaaaccattgccttaagtagctggctgaaggtaatgtcgggatagataaccacagaagccatttgggctgcagtgttaagtgttagaatacataatgaggaactgaggacgtacccagtgcgtttggttggagtggggcaggatgttaattcagataaaacattgcagaaagcaacatctgtatatgtctggaggacaagtagagtcaggtgggggaagtgtctttgggcagagggaaagcatgtgcaaaggactgggagctacaggtaactaataagctaacagataagacaaagttagatctttgtgtttttaattttttaaaaattttaaaatatcatttatttatttttgagacagagtttcactctgttgcccaggctggagtgcagtggcaagatctcagctcactgcaaactctatggccccaggttcaagttgattcttgtgcctcagcctcccaagttacctgggactacaggcatgcgccatcatgcctggctaattttttttttttttttttgtatttttagtagagacaggatttcaccatgttggcctcaaactcctgacctcaagtgctccacctgccttggcctcccaaagtgctgggattacaggcgtgagccactgtgcccggcccagttttaatattttagtggtatgttcagtggaaagaatgggttaaggatggtttagagcaaggggggatttcattcaggaaagcttgagaaaaaaatggctctttgtttctttgtatcactgcagcgggaaaaaggccccaatagggagtggttctgcagatgccttttcagaggttttggtgacatggcagatgtgcttggttaacctcaacttcttctggtcccttggtcctctccagtgactcaaacaagtgtctgagaacagtttttcttagcattcactctttgtcctcactaaggcaggaccttgtcacagggtatggtagggaatgtgatctgaacagatacacctcattggtatttgttatccaacagcagttatgatgatcgttcgtccgaccggagggtgtatgaccggcgatactgtggcagctacagacgcaacgattatagccgggatcggggagatgcctactatgacacagactatcggcattcctatgaatatcagcgggagaacagcagttaccgcagccagcgcagcagccggaggaagcacagacggcggaggaggcgcagccggacatttagccgctcatcttcggtgagtgccagcccaggcccttcctctccccactcttctgcaggccctctaggactctggtaagtgagcagtatccttgttctcagctgaacattggggcatgaacactgaggtgggcactgagtttgcctactttcttggaagctctccgactcttgaagggccctggatctgctttggagatggatgggcacggagcatttgtgacccccagtgctctccctggcatgttgggcttattgtgttgggagcagcttctccgccccagcggcctccactctttaatggggaccttgcttgttgaactgcctttttcccccaagccctgggctctgtagcccccttggcaggcgggcttgggtgggggcaaggggagatctgtgtctgcccggaagggcattgtgtagagcatggggttgtgggtgacattggcaacaacaccacttctctgctctgcccatgcctctccctgttcctgttttgggtttggggacttgggattatgcgccgctctctcttctcacaccgatccaccttactgcatctgacgtgttcccttccactgtcccccatcatcgtctgtcccccctggctgggcgcctgtgaccggtgacccctctaccacccaccccgcctccctccggcccccggctccgaacactgggcttggttcggaaccccccctgccccccgacagcagcacagcagccggagagccaagagtgtagaggacgacgctgagggccacctcatctaccacgtcggggactggctacaagagcgatgtacaagccaaatcgtaacaatcctatagcctgtaatggtcccatagccatcctaacgtcccaagccaacctgagtcacagcctcttgccttttgctcagtggtgttgtttatctgggtggtttgagttgctgttgagaattgacctctgttgtccactcccagctctcacggcccctgggttaaaggtggtgggaatcacgcaggggttttcttcccctgtgaccatctgtatctgttccccttccttcatctccaccccaggttgctgtccccttttttcttccaactcagctcattccccaccttctctccctccctctccccgaccctgctctctttcatttcagatgaaatcgttagcaccttaggagaggggaccttcggccgagttgtacaatgtgttgaccatcgcaggtaactgtcagtccctccctactatgtggggctaaagagatggttggggttatatggggcttttttgctaattaacctgaggtagaatttcttagtccccctacagccctgttcattttgagacattcttgagaacccagcaaaagcctctcctgccaacttacaggggtggggctcgagttgccctgaagatcattaagaatgtggagaagtacaaggaagcagctcgacttgagatcaacgtgctagagaaaatcaatgagaaagaccctgacaacaagaagtaagcaagcaaggaagtgtgtagggaggctgagagccccaacccctacacgggagagattcctagacctggtttaggcagacagggggagtcctagaccttctcatccattcatcttttgttcatcatcttagagtagtagaacatcagggtaggaaagggggggggcccatgacattccttaatccactccccttgttttcagggaagttagattgctctgtcgtttgaccccagcctagaatggcttctgtagagatataccctggacatagccctaggactgagcatcaacctcatcgaacaatgaattaagcttttataagtagaactatgctgataaggccacagacatttatgcagtaatattttgttcacatacattcacagtccaaaaatagaataaggactttaagccaagatatgaggccagtaggtataaataggagcccagtggtagctagtggtgaatgaatatcaggagttgggctggggtttgggatttaggatggacagtttgatgattccaggatctatactgtttggagcttggcactccacagctcttccagcaaatagtttttgaactatttaaaatgacgcaggaagatattttgaaacttgggctgggcatggtggctcacgcctgtaatcccagcactttggaggccgaggcgggtggatcacaatgtcaggagttcgagaccagcctggccaatatggtgaaaccccgtctgtactaaaaataaaaaaattagccgggcatggtggtggacaactgtagtcccagctacttggaaggctgaggcaggagaatcgcttgaacaggaggcagaggttgcagtgagccaagatcgcgccactgcactccagcctgggtgacagagtgagacgccatctcaaaaacaaaaaagaaaaaaacatggcagatagtaccatttctttcttgggtctggtagaggctactccttagctgaactgaattttggtgttagtactagctggcatggttttacacaagttatgtggaatcaacagctatgaagtacctccttgttggatggctgatgggcagatgggagctcatcagacaatcccccttcccccatctctcttctcagcctctgtgtccagatgtttgactggtttgactaccatggccacatgtgtatctcctttgagcttctgggccttagcaccttcgatttcctcaaagacaacaactacctgccctaccccatccaccaagtgcgccacatggccttccagctgtgccaggctgtcaagtgtgagtggggtgggccgaagtggactctggggcagtccctcccttcattggatctcttctgtcggttgtgcactggtgaagcccctaaacagtcagctgtctgttatctgcagttctttgatttactgtcatcttgaaacgtcttctgacttaactccttgactgatgtctttatcgtcactgattgctcttactctacacctagcctagcagcagctagaagagaaagcctttggaatcaaagcacttaattaccctccccttttcctttctccctttcttgggaaagcatcagtcagacagcaaacataaagagacaaaaatacactccttagggtaaaggcttacatttgtctgggatgagatgttcattcacagcaaaggagatgggaacacagagtatgtagttcagctaaggggcaaggtggggaattcaaagaaatatatcattcctctggagtcatcaaaataatacagtttcacagaattgagttaacataatgccagctagacacatgtcaaagtgtgtacacactacaactcaaagcaaactttttttttttttacatcagttgagctacatatgtatcttacattagaaagagcagagcttcttagaccaggcattccatttagacgtagagcggaaagcagccctagtgattctggacctgtctcctcactggctttgccctaggtaaccaggcctggggtcagctgataccagttagctctggccacctgcaccaagcctgacctggccttctcccctacagtcctccatgataacaagctgacacatacagacctcaagcctgaaaatattctgtttgtgaattcagactatgagctcacctacaacctagagaaggtaagatggatagggtctgcccttggttactgggggcaggcagctgcaccactttgccttctgccgagccctttgttttctcccttttatttcgtcctcccacattttctccctgactggctccaactgggtaaaactaaataggttgaaagggagaaatctcttaagaagacttaaattgggagataacttgtacaggggacttcagataactttccagtagagtgaaagtttttaaggttcttggtttgggctttatttatttatttatttatttatttatttatttatttgagacagagtctcactctgttgcccagactgatgtgcagtggcataatctcggctcactgcaacctctacctcccaggtttaagtgattctcctgcctcagcctctggagtagctggggttacaggcacccgccaccacgcctggctagtttttgtgtttttagtagagatggggttttgccatgttggccaggctggtctcgaacccctaacctcaggtgatctgcctgtctcgggctcccagaatgctgggattacgggtgtgagccacagtgcctggcccggacttcttttttgagatgtgtatttctgtgaggtaggaaagctcggctcctttgactaactggagataatgaagatctcagacctaaagaagctctccctcctttgacccctttgctacatgttacatatttttagagaaactcctttgctacatgttacattttttcagaaaaacccatttgctacatgttacattttttcagaaaaacccatttgctacatgttacatgttttcagaataactatatatctggcacttgagtgtagtcttcagatgcttacagatgtgcacgctgttctataatcatttcttaatccttttgctcccctacttctaaaagtattatgctggatgtggtggaagctgtaaaacaggaatttcccaggcttgtaaggccaggcaggagatcacagtaccttttttaaggctcagaagcaatgtagagaatactggtggaatctcagtctgggtgcagaggttcatcctctttctcctctgctcctagaagcgagatgagcgcagtgtgaagagcacagctgtgcgggtggtagactttggcagtgccacctttgaccatgagcaccatagcaccattgtctccactcgccattaccgagcaccagaagtcatccttggtaagggagggcaaggctgtccaagtgtgtgagatgatgtgagggtggggccacctaagcctcataacaccttttccctcccattctcacccagagttgggctggtcacagccttgtgatgtgtggagtataggctgcatcatctttgaatactatgtgggattcaccctcttccaggtaagtgatgggatgtcttacttgactgcctggcattcttctacctggttcctttgttttctgctgaggacctgcctactcagttctccacattgcctgccttcctggcagctgatccttagcatgcccttttcagtcccatgctcagtttctgtttttgtttcccagacccatgacaacagagagcatctagccatgatggaaaggatcttgggtcctatcccttcccggatgatccgaaagacaaggtgaaccttgagggggcactagttaactcttttccttttctctccacagaattggtctatttcacatcattttcttttttctttgatacctcctctccccccagttactttcagatggggaaataagggaattgtaacaagggtgaccttctgattcctcaacctccccttcccctctagaaagcagaaatatttttaccggggtcgcctggattgggatgagaacacatcagctgggcgctatgttcgtgagaactgcaaaccgctgcgggtgagctgggctcgggataaatagtgcccaccgtccagaagtcacttccttcttagggtggttgccccctggaatgctcttcaacaagccagagggttaggaaaggaggggaggaaagctgaaagaagacatctttggtcaacagaggaaacataagagggagtggttttgcggagggaaggaggttagacagcctaaccttgagacaaccagagatcaaagcaatgtcctggattctttaggtcagacagaaaagaataaactacccttgaagagcttacattttaatgaggaactaaagaagattcatgaagttgacaaggatatacaagtagaaagaactttcaaagattatggagtaattgtgctagagggaaggtaggttgagctataatatcagaaacgttggtcctggtgtggtcgtggttaggtagccttcaaattggttgcaagcagagccttggttctccaagaatgaaaggtagggtcttgaagaaggcagggtttgtaaggcatcctgcctcacctttttcctgccctcctccaccagcggtatctgacctcagaggcagaggaacaccaccagctcttcgatctgattgaaagcatgctagagtatgaaccagctaagcggctgaccttgggtgaagcccttcagcatcctttcttcgcccgccttcgggctgagccgcccaacaagttgtgggactccagtcgggatatcagtcggtgacgatcaggccctgggcccccctgcatcttttatagcagtgggtgtccagtccaggacactggtgcttttttatacaagagaacgagccagagttcactccttcctcctggctctctatatacctgtgaatatgtgaaatagtgtaaatatgaaagaacttgtacctatcacttcaacccctgccttgtacataatactattccatccacacagtttccaccctcacctgccccctcatacggagttggatgggggccgagtgaggtaaccaggtggcatctaccccatgttttataaggaattttgtacagtctttgtgaaataaaataacgtgcttcatttga